(19) United States
(12) Patent Application Publication
(10) Pub. No.: US 2022/0339256 A1
(43) Pub. Date: Oct. 27, 2022

(54) COMPOSITIONS AND METHODS FOR TREATING HEPATITIS B VIRUS (HBV) INFECTION

(71) Applicant: Vir Biotechnology, Inc., San Francisco, CA (US)

(72) Inventors: Phillip S. Pang, San Francisco, CA (US); Anna Bakardjiev, San Francisco, CA (US); Lynn E. Connolly, San Francisco, CA (US)

(21) Appl. No.: 17/611,063

(22) PCT Filed: May 12, 2020
(86) PCT No.: PCT/US2020/032525

§ 371 Date: Nov. 12, 2021

Related U.S. Application Data
(60) Provisional application No. 62/846,927, filed on May 13, 2019.
(60) Provisional application No. 62/893,646, filed on Aug. 29, 2019.
(60) Provisional application No. 62/992,785, filed on Mar. 20, 2020.
(60) Provisional application No. 62/994,177, filed on Mar. 24, 2020.
(60) Provisional application No. 63/009,910, filed on Apr. 14, 2020.

(51) Int. Cl. A61K 38/21; A61P 31/20; A61K 31/7105; A61K 47/60; A61K 31/675; A61K 31/513; A61K 31/522; A61K 31/427; A61K 31/198; A61K 31/7072; A61K 31/506

(52) U.S. Cl. CPC A61K 38/212; A61P 31/20; A61K 31/7105; A61K 47/60; A61K 31/675; A61K 31/513; A61K 31/522; A61K 31/427; A61K 31/198; A61K 31/7072; A61K 31/506

(57) ABSTRACT

The present disclosure provides methods for treating HBV infection using an siRNA that targets an HBV gene. In some embodiments, the method for treating HBV involves co-administration of siRNA with PEG-INFα.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930485_405USPC_Sequence_Listing.txt. The text file is 6.52 KB, was created on Nov. 12, 2021, and is being submitted electronically via EFS-Web.

### BACKGROUND

Chronic hepatitis B virus (HBV) infection remains an important global public health problem with significant morbidity and mortality (Trepo C., A brief history of hepatitis milestones, Liver International 2014, 34(1):29-37). According to the World Health Organization (WHO) an estimated 257 million people are living with chronic HBV infection worldwide (WHO, 2017; Schweitzer A, et al., Estimations of worldwide prevalence of chronic hepatitis B virus infection: a systematic review of data published between 1965 and 2013, The Lancet 2015, 387(10003):1546-1555). Over time, chronic HBV infection leads to serious sequelae including cirrhosis, liver failure, hepatocellular carcinoma (HCC), and death. Almost 800,000 people are estimated to die annually due to sequelae associated with chronic HBV infection (Stanaway J D, et al., The global burden of viral hepatitis from 1990 to 2013: findings from the Global Burden of Disease Study 2013, The Lancet 2016, 388(10049):1081-1088).

HBV prevalence varies geographically, with a range of less than 2% in low to greater than 8% in high prevalence countries (Schweitzer et al., 2015). In high prevalence countries, such as those in sub-Saharan Africa and East Asia, transmission occurs predominantly in infants and children by perinatal and horizontal routes. In more industrialized countries, new infections are highest among young adults and transmission occurs predominantly via injection drug use and high-risk sexual behaviors. The risk of developing chronic HBV infection depends on the age at the time of infection. While only approximately 10% of people infected as adults develop chronic HBV infection, 90% of infants infected perinatally or during the first 6 months of life, and 20-60% of children infected between 6 months and 5 years of age, remain chronically infected. Twenty-five percent of people who acquire HBV during infancy and childhood will develop primary liver cancer or cirrhosis during adulthood.

HBV is a DNA virus that infects, replicates, and persists in human hepatocytes (Protzer U, et al., Living in the liver: hepatic infections, Nature Reviews Immunology 201, 12: 201-213). The small viral genome (3.2 kb), consists of partially double-stranded, relaxed-circular DNA (rcDNA) and has 4 open reading frames encoding 7 proteins: HBcAg (HBV core antigen, viral capsid protein), HBeAg (hepatitis B e-antigen), HBV Pol/RT (polymerase, reverse transcriptase), PreS1/PreS2/HBsAg (large, medium, and small surface envelope glycoproteins), and HBx (HBV×antigen, regulator of transcription required for the initiation of infection) (Seeger C, et al., Molecular biology of hepatitis B virus infection, Virology, 2015, 479-480:672-686; Tong S, et al., Overview of viral replication and genetic variability, Journal of Hepatology, 2016, 64(1):S4-S16).

In hepatocytes, rcDNA, the form of HBV nucleic acid that is introduced by the infection virion, is converted into a covalently closed circular DNA (cccDNA), which persists in the host cell's nucleus as an episomal chromatinized structure (Allweiss L, et al., The Role of cccDNA in HBV Maintenance, Viruses 2017, 9: 156). The cccDNA serves as a transcription template for all viral transcripts (Lucifora J, et al., Attacking hepatitis B virus cccDNA—The holy grail to hepatitis B cure, Journal of Hepatology 2016, 64(1): S41-S48). Pregenomic RNA (pgRNA) transcripts are reverse transcribed into new rcDNA for new virions, which are secreted without causing cytotoxicity. In addition to infectious virions, infected hepatocytes secrete large amounts of genome-free subviral particles that may exceed the number of secreted virions by 10,000-fold (Seeger et al., 2015). Random integration of the virus into the host genome can occur as well, a mechanism that contributes to hepatocyte transformation (Levrero M, et al., Mechanisms of HBV-induced hepatocellular carcinoma, Journal of Hepatology 2016, 64(1): S84-S101). HBV persists in hepatocytes in the form of cccDNA and integrated DNA (intDNA).

Hepatitis B infection is characterized by serologic viral markers and antibodies (FIG. 1). In acute resolving infections, the virus is cleared by effective innate and adaptive immune responses that include cytotoxic T cells leading to death of infected hepatocytes, and induction of B cells producing neutralizing antibodies that prevent the spread of the virus (Bertoletti A, 2016, Adaptive immunity in HBV infection, Journal of Hepatology 2016, 64(1): S71-S83; Maini M K, et al., The role of innate immunity in the immunopathology and treatment of HBV infection, Journal of Hepatology 2016, 64(1): S60-S70; Li Y, et al., Genome-wide association study identifies 8p21.3 associated with persistent hepatitis B virus infection among Chinese, Nature Communications 2016, 7:11664). In contrast, chronic infection is associated with T and B cell dysfunction, mediated by multiple regulatory mechanisms including presentation of viral epitopes on hepatocytes and secretion of subviral particles (Bertoletti et al., 2016; Maini et al., 2016; Burton A R, et al., Dysfunctional surface antigen specific memory B cells accumulate in chronic hepatitis B infection, EASL International Liver Congress, Paris, France 2018). Thus, the continued expression and secretion of viral proteins due to cccDNA persistence in hepatocytes is considered a key step in the inability of the host to clear the infection.

Chronic HBV infection is a dynamic process reflecting the interaction between HBV replication and host immune responses. The laboratory hallmark of chronic HBV infection is persistence of HBsAg in the blood for greater than six months, and a lack of detectable anti-HBs. Chronic infection is divided into four stages based on HBV markers in blood (HBsAg, HBeAg/anti-HBe, HBV DNA), and liver disease based on biochemical parameters (alanine aminotransferase, “ALT”), as well as fibrosis markers (noninvasive or based on liver biopsy) (EASL, 2017). Overall, across the various phases of chronic HBV infection, only a minority of patients (less than 1% per year) clear the disease as measured by HBsAg seroclearance.

A sterilizing cure for HBV would involve complete eradication of HBV DNA or permanent transcriptional silencing of HBV DNA, without a risk of recurrence. Potential therapies that could eliminate or permanently silence the cccDNA/intDNA carry the risk of damaging or altering the transcription of the human chromosomal DNA.

In contrast, a functional cure is defined as life-long control of the virus. Patients with a history of acute hepatitis B who seem to be cured have ˜40% risk for HBV recurrence if undergoing immunosuppression. In this way, functional cure is part of the natural history of HBV infection. Potential therapies that provide a functional cure may require immunomodulation. This is because chronic HBV infection leads to B and T cell exhaustion, potentially due to expression of HBV antigens (tolerogens), which could prevent efficacy of immune modulators.

Currently, there are two main treatment options for patients with chronic HBV infection: treatment with nucleoside/nucleotide reverse transcriptase inhibitors (NRTIs) and pegylated interferon-alpha (PEG-IFNα) (Liang T J, et al., Present and Future Therapies of Hepatitis B: From Discovery to Cure, Hepatology 2015, 62(6):1893-1908). NRTIs inhibit the production of infectious virions, and often reduce serum HBV DNA to undetectable. However, NRTIs do not directly eliminate cccDNA, and therefore, transcription and translation of viral proteins continues. Consequently, expression of viral epitopes on hepatocytes, secretion of subviral particles, and immune dysfunction remain largely unaffected by NRTI therapy. As a consequence, this necessitates prolonged, often lifelong therapy (however, less than half of patients remain on therapy after 5 years). NRTI therapy leads to a loss of serum HBsAg at a rate of ˜0-3% per year. Furthermore, while NRTI therapy reverses fibrosis and reduces the incidence of HCC, it does not eliminate the increased risk of HCC that HBV infection confers.

In contrast, PEG-IFN can induce long-term immunological control, but only in a small percentage of patients (<10%) (Konerman M A, et al., Interferon Treatment for Hepatitis B, Clinics in Liver Disease 2016, 20(4): 645-665). PEG-IFN typically requires 48 weeks of therapy and the duration-dependent side effects are significant. In studies evaluating PEG-IFNα for the treatment of chronic hepatitis C infection, 12- or 24-week regimens were associated with lower rates of serious adverse events, grade 3 adverse events, and treatment discontinuations than those observed in trials evaluating 48-week regimens (Lawitz E, et al., Sofosbuvir for previously untreated chronic hepatitis C infection, N Engl J Med. 2013, 368(20): 1878-1887); Hadziyannis S J, et al., Peginterferon-alpha2a and ribavirin combination therapy in chronic hepatitis C: a randomized study of treatment duration and ribavirin dose, Ann Intern Med. 2004, 140(5): 346-355; Fried MW, et al., Peginterferon alfa-2a plus ribavirin for chronic hepatitis C virus infection, N Engl J Med. 2002, 347(13): 975-982). The high variability of response, in combination with an unfavorable safety and side effect profile, make a significant number of patients ineligible or unwilling to undergo PEG-IFNα treatment.

The failure of NRTI therapy to eradicate the virus, and the limitations of PEG-IFNα therapy, highlight the clinical need for new HBV therapies that are effective, well tolerated, and do not require lifelong administration.

### SUMMARY

In some aspects, the present disclosure relates to compositions and methods of treating HBV with siRNA, in particular HBV02. For example, in accordance with some embodiments, a method of treating an HBV infection in a subject by administering an siRNA is provided, wherein the siRNA has a sense strand that comprises SEQ ID NO: 5 and an antisense strand that comprises SEQ ID NO: 6. In some embodiments, the method of treating further comprises administering to the subject a pegylated interferon-alpha (PEG-INFα). In some embodiments the PEG-INFα is administered before, concurrently, or after the siRNA HBV02 is administered. In some embodiments, the HBV infection is chronic. In some further embodiments, the subject is administered a nucleoside/nucleotide reverse transcriptase inhibitor (NRTI). In some embodiments the NRTI is administered before, concurrently, or after the HBV02 is administered. In some embodiments the NRTI is administered for 2 to 6 months prior to the HBV02.

In some aspects, the present disclosure also provides a siRNA for use in the treatment of an HBV infection in a subject, wherein the siRNA is HBV02 and has a sense strand that comprises SEQ ID NO: 5 and an antisense strand that comprises SEQ ID NO: 6. In some additional embodiments, the siRNA HBV02 is administered to a subject that is also administered a PEG-INFα. In some embodiments, the PEG-INFα is administered before, concurrently, or after the siRNA HBV02 is administered. In some embodiments, the HBV infection is chronic. In some further embodiments, the subject is administered a NRTI. In some embodiments the NRTI is administered before, concurrently, or after the HBV02 is administered. In some embodiments the NRTI is administered for 2 to 6 months prior to the HBV02.

In some further aspects, the present disclosure provide for the use of an siRNA in the manufacture of a medicament for the treatment of an HBV infection, wherein the siRNA is HBV02 and has a sense strand that comprises SEQ ID NO: 5 and an antisense strand that comprises SEQ ID NO: 6. In some embodiments, the use of the siRNA HBV02 is for use with PEG-IFNα. In some embodiments, the siRNA HBV02 is for use with PEG-IFNα and an NRTI.

In some of the aforementioned embodiments, the dose of the siRNA HBV02 is 0.8 mg/kg, 1.7 mg/kg, 3.3 mg/kg, 6.7 mg/kg, 10 mg/kg, or 15 mg/kg. In some of the aforementioned embodiments, the dose of the siRNA HBV02 is from 20 mg to 900 mg. In some of the aforementioned embodiments, the dose of the siRNA HBV02 is 20 mg, 50 mg, 100 mg, 150 mg, 200 mg, 250 mg, 300 mg, 400 mg, or 450 mg. In some of the aforementioned embodiments, the HBV02 is administered weekly. In some of the aforementioned embodiments, more than one dose of the siRNA is administered. In some of the aforementioned embodiments, two, three, four, five, six, or more doses of the siRNA are administered with each dose separated by 1, 2, 3, or 4 weeks. In some of the aforementioned embodiments, six 200-mg doses of the siRNA are administered. In some of the aforementioned embodiments, two 400-mg doses of the siRNA are administered. In some of the aforementioned embodiments, the siRNA is administered via subcutaneous injection; for example, in some embodiments, administering the siRNA HBV02 includes administering 1, 2, or 3 subcutaneous injections per dose.

In some of the aforementioned embodiments, the dose of PEG-IFNα is 50 μg, 100 μg, 150 μg, or 200 μg. In some of the aforementioned embodiments, the PEG-IFNα is administered weekly. In some of the aforementioned embodiments, the PEG-IFNα is administered via subcutaneous injection.

In some of the aforementioned embodiments, the NRTI is tenofovir, tenofovir disoproxil fumarate (TDF), tenofovir alafenamide (TAF), lamivudine, adefovir, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, ganciclovir, besifovir (ANA-380/LB-80380), or tenofvir-exaliades (TLX/CMX157).

In some of the aforementioned embodiments, the subject is HBeAg negative. In some embodiments, the subject is HBeAg positive.

In some aspects of the disclosure, a kit is provided comprising: a pharmaceutical composition comprising an siRNA according to any of the preceding embodiments, and a pharmaceutically acceptable excipient; and a pharmaceutical composition comprising PEG-IFNα, and a pharmaceutically acceptable excipient. The kit may also contain a NRTI, and a pharmaceutically acceptable excipient.

### BRIEF DESCRIPTION OF THE DRAWINGS

FIG. 1 depicts characteristics of acute and chronic Hepatitis B infections.

FIG. 2 depicts characteristics of chronic Hepatitis B infection. The disease is divided into 4 phases based on HBeAg status and laboratory or radiographic evidence of liver disease. Heterogeneity of disease could be due to differences in virus (e.g., HBV genotypes, mutations), host (e.g., immune responses, age at inflection, number of infected hepatocytes), and other factors (e.g. co-infections (HDV, HCV, HIV), intercurrent infections, co-morbidities).

FIG. 3 depicts the single ascending dose design for Part A of Example 2. ^{a}Subject discharge occurs after all assessments are completed on day 2.

FIG. 4 depicts the multiple ascending dose design for Parts B and C of Example 2. ^{a}Additional HBsAg monitoring is required for subjects with HBsAg levels with a >10% decrease from the Day 1 predose level at the Week 16 visit. Visits occur every 4 weeks starting at Week 20 up to Week 48 or until the HBsAg level returns to ≥90% of the Day 1 perdose level.

FIG. 5A to FIG. 5B depict the cohort dosing schedule for Parts A, B, and C of Example 2, including optional cohorts and floater subjects. *Up to 8 subjects for Part A and up to 16 subjects total for Parts B/C may be added as part of an expansion of an existing cohort or cohorts if further data are required (the allocation of the floater subjects in Parts B/C is not required to be distributed evenly; the total combined n for Parts B/C does not exceed 48 subjects). **The doses designated in Parts B/C schedule are indicative of a single dose of HBV02 or placebo; subjects receive up to 2 doses total.

FIG. 6A to FIG. 6D depict the cohort dosing schedule for Part D of Example 2. FIG. 6A shows the design for cohort 1d; FIG. 6B shows the design for cohort 2d; FIG. 6C shows the design for cohort 3d; and FIG. 6D shows the design for cohort 4d.

FIG. 7A to 7B depict the cohort dosing schedule for Parts A, B, C, and D of Example 2 including optional cohorts and floater subjects (dashed lines on FIG. 7A).

FIG. 8 depicts the cohort dosing schedule for Parts A, B, and C of the study in Example 3.

FIG. 9A to 9C depict studies generating preliminary data in Example 3. FIG. 9A illustrates the study design at the time dosing was completed for Part A cohorts 1 through 5 (50 mg, 100 mg, 200 mg, 400 mg, 600 mg) and for Part B cohorts 1 through 2 (50 mg, 100 mg). FIG. 9B illustrates the Part A completed dosing for cohorts 1 through 5, and the withdrawal of subjects in the different cohorts. FIG. 9C depicts the Part B completed dosing for cohorts 1 through 2, and the withdrawal of subjects in the different cohorts.

FIG. 10A to FIG. 10B depict ALT levels for subjects in cohorts 1 through 4 of Part A of Example 3. FIG. 10A shows ALT levels for subjects that received 50 mg (cohort 1a) or 100 mg (cohort 2a) of HBV002. FIG. 10B shows ALT levels for subjects that received 200 mg (cohort 3a) or 400 mg (cohort 4a) of HBV002. One subject in the 200-mg cohort had an ALT at ULN on Day 29 associated with strenuous exercise and high creatinine kinase (CK: 5811 U/L). Two subjects in the 400-mg cohort had ALT values above ULN on Day 1 prior to dosing; one of these subjects admitted to strenuous exercise, had high CK (20,001 U/L), and withdrew on Day 2 unrelated to adverse events, and the ALT of the other subject resolved by Day 8 without intervention.

FIG. 11 depicts ALT levels for subjects in Part B of Example 3 that received 50 mg (cohort 1b) or 100 mg (cohort 2b) of HBV002. One female subject in the 100-mg cohort exhibited a grade 1 ALT elevation at Week 8.

FIG. 12A to 12C depict antiviral activity in Part B cohorts 1b (50 mg) and 2b (100 mg) of Example 3 as measured by change in HBsAg levels. FIG. 12A shows change in HBsAg levels among active and placebo subjects. FIG. 12B shows change in HBsAg levels among only active subjects. FIG. 12C shows change in HBsAg levels (mean change from Day 1 in HBsAg following administration of HBV02) among subjects in the 50 mg (cohort 1b) and 100 mg (cohort 2b) cohorts.

FIG. 13A to FIG. 13E show ALT levels in chronic HBV patients in Example 3 through Week 16 (n=32). FIG. 13A shows ALT levels for all patients, and these results are shown separately for different HBV02 dose levels in FIGS. 13B (20 mg), 13C (50 mg), 13D (100 mg), and 13E (200 mg).

FIG. 14 shows treatment-emergent post-baseline ALT elevations in healthy volunteers with normal ALT at baseline, corresponding to Example 3. The highest treatment-emergent post-baseline ALT elevation, expressed relative to upper limit of normal (ULN), is shown n the y-axis. Dose of HBV01 or HBV02 is shown on the x-axis. *Approximate mg/kg dose based on an average adult weight of 60 kg; fixed doses of HBV02 ranged from 50-900 mg.

FIG. 15A to FIG. 15B show plasma concentration vs time profiles for HBV02 (A) and AS(N-1)3′ HBV02 (B) after a single subcutaneous dose in healthy volunteers, corresponding to Example 3.

FIG. 16 shows plasma AUC₀₋₁₂ for HBV02 following a single subcutaneous dose in healthy volunteers, corresponding to Example 3. Dose proportionality was observed from 50 mg to 900 mg.

FIG. 17 shows plasma Cₘₐₓ for HBV02 following a single subcutaneous dose in healthy volunteers, corresponding to Example 3. Dose proportionality was observed from 50 mg to 900 mg.

FIG. 18 shows plasma PK parameters for HBV02 and AS(N-1)3′ HBV02 after a single SC dose in healthy volunteers in Example 3. Time parameters are expressed as median (quartile [Q]1, Q3); all other data are presented as mean (% coefficient of variation [CV]). Due to short HBV02 half-life (t_{1/2}) and PK sampling schedule limitations, terminal phase was not adequately characterized; therefore, apparent clearance and t_{1/2}were not reported. ^{a}Excludes 1 volunteer who received partial dose; ^{b}includes PK from replacement volunteer; ^{c}measurable in 3/6 volunteers; AUC, area under curve; AUC₀₋₁₂, AUC from time 0 to 12 hr; AUCₗₐₛₜ, AUC from time of dosing to last measurable time point; BLQ, below limit of quantitation; Cₘₐₓ, maximum concentration; CV, coefficient of variance; MR, metabolite-to-parent ratio; NC, not calculable; Tₘₐₓ=time of Cₘₐₓ; Tₗₐₛₜ, last measurable time.

FIG. 19A to 19B show urine concentration vs time profiles for HBV02 (A) and AS(N-1)3′ HBV02 (B) after a single subcutaneous dose in healthy volunteers, corresponding to Example 3.

FIG. 20 shows plasma PK parameters for HBV02 and AS(N-1)3′ HBV02 in healthy volunteers in Example 3. All PK parameters are expressed as mean (CV%). ^{a}Excludes 1 volunteer who received partial dose; ^{b}includes PK from replacement volunteer; ^{c}AUC₀₋₂₄ is extrapolated; AUC₀₋₂₄, AUC from time 0 to 24 hr; CLR, total renal clearance; feo-24, fraction excreted from time 0 to 24 hr; NC, not calculable.

FIG. 21A to 21B depict antiviral activity in Parts B and C of Example 3, measured by change in HBsAg levels. FIG. 21A shows change in HBsAg levels in log scale.

FIG. 22 depicts HBsAg change from baseline by dose of HBV02, or for placebo, for Example 3. Follow-up data available for all placebo patients through Week 16, compared to 24 weeks for treatment groups.

FIG. 23 depicts individual maximum HBsAg change from baseline for Example 3. Error bars represent median (interquartile range).

FIG. 24 shows individual HBsAg change from baseline at Week 24 for Example 3. Error bars represent median (interquartile range).

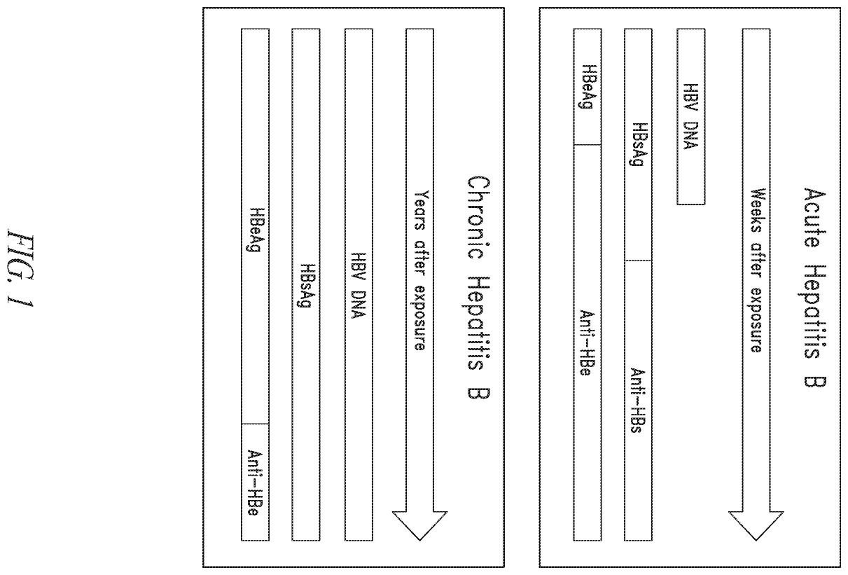

### DETAILED DESCRIPTION

The instant disclosure provides methods, compositions, and kits for use in treating hepatitis B virus (HBV) infection, wherein a small interfering RNA (siRNA) molecule that targets HBV is administered. In some embodiments, the siRNA molecule is administered with a pegylated interferon-2a (PEG-IFNα) therapy or is administered to a subject that has received or will receive a PEG-IFN-α therapy. In some embodiments, the methods, compositions, and kits disclosed herein are used to treat chronic HBV infection.

### I. Glossary

Prior to setting forth this disclosure in more detail, it may be helpful to an understanding thereof to provide definitions of certain terms to be used herein. Additional definitions are set forth throughout this disclosure.

In the present description, the term “about” means±20% of the indicated range, value, or structure, unless otherwise indicated.

The term “comprise” means the presence of the stated features, integers, steps, or components as referred to in the claims, but that it does not preclude the presence or addition of one or more other features, integers, steps, components, or groups thereof. The term “consisting essentially of” limits the scope of a claim to the specified materials or steps and those that do not materially affect the basic and novel characteristics of the claimed invention.

It should be understood that the terms “a” and “an” as used herein refer to “one or more” of the enumerated components. The use of the alternative (e.g., “or”) should be understood to mean either one, both, or any combination thereof of the alternatives, and may be used synonymously with “and/or”. As used herein, the terms “include” and “have” are used synonymously, which terms and variants thereof are intended to be construed as non-limiting.

The word “substantially” does not exclude “completely”; e.g., a composition which is “substantially free” from Y may be completely free from Y. Where necessary, the word “substantially” may be omitted from definitions provided herein.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning. A “disease” is typically manifested by distinguishing signs and symptoms, and causes the human or animal to have a reduced duration or quality of life.

As used herein, the terms “peptide,” “polypeptide,” and “protein” and variations of these terms refer to a molecule, in particular a peptide, oligopeptide, polypeptide, or protein including fusion protein, respectively, comprising at least two amino acids joined to each other by a normal peptide bond, or by a modified peptide bond, such as for example in the cases of isosteric peptides. For example, a peptide, polypeptide, or protein may be composed of amino acids selected from the 20 amino acids defined by the genetic code, linked to each other by a normal peptide bond (“classical” polypeptide). A peptide, polypeptide, or protein can be composed of L-amino acids and/or D-amino acids. In particular, the terms “peptide,” “polypeptide,” and “protein” also include “peptidomimetics,” which are defined as peptide analogs containing non-peptidic structural elements, which are capable of mimicking or antagonizing the biological action(s) of a natural parent peptide. A peptidomimetic lacks classical peptide characteristics such as enzymatically scissile peptide bonds. In particular, a peptide, polypeptide, or protein may comprise amino acids other than the 20 amino acids defined by the genetic code in addition to these amino acids, or it can be composed of amino acids other than the 20 amino acids defined by the genetic code. In particular, a peptide, polypeptide, or protein in the context of the present disclosure can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain, or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide,” “polypeptide,” or “protein” in the context of the present disclosure in particular also include modified peptides, polypeptides, and proteins. For example, peptide, polypeptide, or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation, or ubiquitination. These modifications are fully detailed in the literature (Proteins Structure and Molecular Properties, 2nd Ed., T. E. Creighton, New York (1993); Post-translational Covalent Modifications of Proteins, B. C. Johnson, Ed., Academic Press, New York (1983); Seifter, et al., Analysis for protein modifications and nonprotein cofactors, Meth. Enzymol. 182:626-46 (1990); and Rattan, et al., Protein Synthesis: Post-translational Modifications and Aging, Ann NY Acad Sci 663:48-62 (1992)). Accordingly, the terms “peptide,” “polypeptide,” and “protein” include for example lipopeptides, lipoproteins, glycopeptides, glycoproteins, and the like.

As used herein a “(poly)peptide” comprises a single chain of amino acid monomers linked by peptide bonds as explained above. A “protein,” as used herein, comprises one or more, e.g., 1, 2, 3, 4, 5, 6, 7, 8, 9 or 10 (poly)peptides, i.e., one or more chains of amino acid monomers linked by peptide bonds as explained above. In particular embodiments, a protein according to the present disclosure comprises 1, 2, 3, or 4 polypeptides.

The term “recombinant,” as used herein (e.g., a recombinant protein, a recombinant nucleic acid, etc.), refers to any molecule (protein, nucleic acid, siRNA, etc.) that is prepared, expressed, created, or isolated by recombinant means, and which is not naturally occurring.

As used herein, the terms “nucleic acid,” “nucleic acid molecule,” and “polynucleotide” are used interchangeably and are intended to include DNA molecules and RNA molecules. A nucleic acid molecule may be single-stranded or double-stranded. In particular embodiments, the nucleic acid molecule is double-stranded RNA molecule.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

As used herein, the term “sequence variant” refers to any sequence having one or more alterations in comparison to a reference sequence, whereby a reference sequence is any of the sequences listed in the sequence listing, i.e., SEQ ID NO:1 to SEQ ID NO:6. Thus, the term “sequence variant” includes nucleotide sequence variants and amino acid sequence variants. For a sequence variant in the context of a nucleotide sequence, the reference sequence is also a nucleotide sequence, whereas for a sequence variant in the context of an amino acid sequence, the reference sequence is also an amino acid sequence. A “sequence variant” as used herein is at least 80%, at least 85%, at least 90%, at least 95%, at least 98%, or at least 99% identical to the reference sequence. Sequence identity is usually calculated with regard to the full length of the reference sequence (i.e., the sequence recited in the application), unless otherwise specified. Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=1 1 and gap extension penalty=1].

A “sequence variant” in the context of a nucleic acid (nucleotide) sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleotide sequence can either result in a change in the respective reference amino acid sequence, i.e., in an amino acid “sequence variant” or not. In certain embodiments, the nucleotide sequence variants are variants that do not result in amino acid sequence variants (i.e., silent mutations). However, nucleotide sequence variants leading to “non-silent” mutations are also within the scope, in particular such nucleotide sequence variants, which result in an amino acid sequence, which is at least 80%, at least 85%, at least 90%, at least 95%, at least 98%, or at least 99% identical to the reference amino acid sequence. A “sequence variant” in the context of an amino acid sequence has an altered sequence in which one or more of the amino acids is deleted, substituted or inserted in comparison to the reference amino acid sequence. As a result of the alterations, such a sequence variant has an amino acid sequence which is at least 80%, at least 85%, at least 90%, at least 95%, at least 98%, or at least 99% identical to the reference amino acid sequence. For example, per 100 amino acids of the reference sequence a variant sequence having no more than 10 alterations, i.e., any combination of deletions, insertions, or substitutions, is “at least 90% identical” to the reference sequence.

While it is possible to have non-conservative amino acid substitutions, in certain embodiments, the substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, and isoleucine, with another; substitution of one hydoxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine, and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Unless otherwise stated, alterations in the sequence variants do not necessarily abolish the functionality of the respective reference sequence, for example, in the present case, the functionality of an siRNA to reduce HBV protein expression. Guidance in determining which nucleotides and amino acid residues, respectively, may be substituted, inserted, or deleted without abolishing such functionality can be found by using computer programs known in the art.

As used herein, a nucleic acid sequence or an amino acid sequence “derived from” a designated nucleic acid, peptide, polypeptide, or protein refers to the origin of the nucleic acid, peptide, polypeptide, or protein. In some embodiments, the nucleic acid sequence or amino acid sequence which is derived from a particular sequence has an amino acid sequence that is essentially identical to that sequence or a portion thereof, from which it is derived, whereby “essentially identical” includes sequence variants as defined above. In certain embodiments, the nucleic acid sequence or amino acid sequence which is derived from a particular peptide or protein is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins, and nucleic acids are thus identifiable to one of ordinary skill in the art. Likewise, sequences “derived from” another sequence are usually identifiable to one of ordinary skill in the art as having its origin in the sequence.

In some embodiments, a nucleic acid sequence or an amino acid sequence derived from another nucleic acid, peptide, polypeptide, or protein may be identical to the starting nucleic acid, peptide, polypeptide, or protein (from which it is derived). However, a nucleic acid sequence or an amino acid sequence derived from another nucleic acid, peptide, polypeptide, or protein may also have one or more mutations relative to the starting nucleic acid, peptide, polypeptide, or protein (from which it is derived), in particular a nucleic acid sequence or an amino acid sequence derived from another nucleic acid, peptide, polypeptide, or protein may be a functional sequence variant as described above of the starting nucleic acid, peptide, polypeptide, or protein (from which it is derived). For example, in a peptide/protein one or more amino acid residues may be substituted with other amino acid residues or one or more amino acid residue insertions or deletions may occur.

As used herein, the term “mutation” relates to a change in the nucleic acid sequence and/or in the amino acid sequence in comparison to a reference sequence, e.g., a corresponding genomic sequence. A mutation, e.g., in comparison to a genomic sequence, may be, for example, a (naturally occurring) somatic mutation, a spontaneous mutation, an induced mutation, e.g., induced by enzymes, chemicals, or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g., in a nucleic acid sequence or in an amino acid sequence. A mutation includes substitution, deletion, and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved, e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

As used herein, the term “coding sequence” is intended to refer to a polynucleotide molecule, which encodes the amino acid sequence of a protein product. The boundaries of the coding sequence are generally determined by an open reading frame, which usually begins with an ATG start codon.

The term “expression” as used herein refers to any step involved in the production of the polypeptide, including transcription, post-transcriptional modification, translation, post-translational modification, secretion, or the like.

Doses are often expressed in relation to bodyweight. Thus, a dose which is expressed as [g, mg, or other unit]/kg (or g, mg, etc.) usually refers to [g, mg, or other unit] “per kg (or g, mg, etc.) bodyweight,” even if the term “bodyweight” is not explicitly mentioned.

As used herein, “Hepatitis B virus,” used interchangeably with the term “HBV” refers to the well-known non-cytopathic, liver-tropic DNA virus belonging to the Hepadnaviridae family. The HBV genome is partially double-stranded, circular DNA with four overlapping reading frames (that may be referred to herein as “genes,” “open reading frames,” or “transcripts”): C, X, P, and S. The core protein is coded for by gene C (HBcAg). Hepatitis B e antigen (HBeAg) is produced by proteolytic processing of the pre-core (pre-C) protein. The DNA polymerase is encoded by gene P. Gene S is the gene that codes for the surface antigens (HBsAg). The HBsAg gene is one long open reading frame which contains three in frame “start” (ATG) codons resulting in polypeptides of three different sizes called large, middle, and small S antigens, pre-S1+pre-S2+S, pre-S2+S, or S. Surface antigens in addition to decorating the envelope of HBV, are also part of subviral particles, which are produced at large excess as compared to virion particles, and play a role in immune tolerance and in sequestering anti-HBsAg antibodies, thereby allowing for infectious particles to escape immune detection. The function of the non-structural protein coded for by gene X is not fully understood, but it plays a role in transcriptional transactivation and replication and is associated with the development of liver cancer.

Nine genotypes of HBV, designated A to I, have been determined, and an additional genotype J has been proposed, each having a distinct geographical distribution (Velkov S, et al., The Global Hepatitis B Virus Genotype Distribution Approximated from Available Genotyping Data, Genes 2018, 9(10):495). The term “HBV” includes any of the genotypes of HBV (A to J). The complete coding sequence of the reference sequence of the HBV genome may be found in for example, GenBank Accession Nos. GI:21326584 and GI:3582357. Amino acid sequences for the C, X, P, and S proteins can be found at, for example, NCBI Accession numbers YP_009173857.1 (C protein); YP 009173867.1 and BAA32912.1 (X protein); YP_009173866.1 and BAA32913.1 (P protein); and YP_009173869.1, YP_009173870.1, YP 009173871.1, and BAA32914.1 (S protein). Additional examples of HBV messenger RNA (mRNA) sequences are available using publicly available databases, e.g., GenBank, UniProt, and OMIM. The International Repository for Hepatitis B Virus Strain Data can be accessed at http://www.hpa-bioinformatics.org.uk/HepSEQ/main.php. The term “HBV,” as used herein, also refers to naturally occurring DNA sequence variations of the HBV genome, i.e., genotypes A-J and variants thereof.

siRNA mediates the targeted cleavage of an RNA transcript via an RNA-induced silencing complex (RISC) pathway, thereby effecting inhibition of gene expression. This process is frequently termed “RNA interference” (RNAi). Without wishing to be bound to a particular theory, long double-stranded RNA (dsRNA) introduced into plants and invertebrate cells is broken down into siRNA by a Type III endonuclease known as Dicer (Sharp, et al., Genes Dev. 15:485 (2001)). Dicer, a ribonuclease-III-like enzyme, processes the dsRNA into 19-23 base pair siRNAs with characteristic two base 3′ overhangs (Bernstein, et al., Nature 2001, 409:363). The siRNAs are then incorporated into RISC where one or more helicases unwind the siRNA duplex, enabling the complementary antisense strand to guide target recognition (Nykanen, et al., 2001, Cell 107:309). Upon binding to the appropriate target mRNA, one or more endonucleases within RISC cleaves the target to induce silencing (Elbashir, et al., Genes Dev. 2001, 15:188).

The terms “silence,” “inhibit the expression of,” “down-regulate the expression of,” “suppress the expression of,” and the like, in so far as they refer to an HBV gene, herein refer to the at least partial reduction of the expression of an HBV gene, as manifested by a reduction of the amount of HBV mRNA which can be isolated from or detected in a first cell or group of cells in which an HBV gene is transcribed and which has or have been treated with an inhibitor of HBV gene expression, such that the expression of the HBV gene is inhibited, as compared to a second cell or group of cells substantially identical to the first cell or group of cells but which has or have not been so treated (control cells). The degree of inhibition can be measured, by example, as the difference between the degree of mRNA expression in a control cell minus the degree of mRNA expression in a treated cell. Alternatively, the degree of inhibition can be given in terms of a reduction of a parameter that is functionally linked to HBV gene expression, e.g., the amount of protein encoded by an HBV gene, or the number of cells displaying a certain phenotype, e.g., an HBV infection phenotype. In principle, HBV gene silencing can be determined in any cell expressing the HBV gene, e.g., an HBV-infected cell or a cell engineered to express the HBV gene, and by any appropriate assay.

The level of HBV RNA that is expressed by a cell or group of cells, or the level of circulating HBV RNA, may be determined using any method known in the art for assessing mRNA expression, such as the rtPCR method provided in Example 2 of International Application Publication No. WO 2016/077321A1 and U.S. Patent Application No. US2017/0349900A1, which methods are incorporated herein by reference. In some embodiments, the level of expression of an HBV gene (e.g., total HBV RNA, an HBV transcript, e.g., HBV 3.5 kb transcript) in a sample is determined by detecting a transcribed polynucleotide, or portion thereof, e.g., RNA of the HBV gene. RNA may be extracted from cells using RNA extraction techniques including, for example, using acid phenol/guanidine isothiocyanate extraction (RNAzol B; Biogenesis), RNeasy RNA preparation kits (Qiagen®), or PAXgene (PreAnalytix, Switzerland). Typical assay formats utilizing ribonucleic acid hybridization include nuclear run-on assays, RT-PCR, RNase protection assays (Melton DA, et al., Efficient in vitro synthesis of biologically active RNA and RNA hybridization probes from plasmids containing a bacteriophage SP6 promoter, Nuc. Acids Res. 1984, 12:7035-56), northern blotting, in situ hybridization, and microarray analysis. Circulating HBV mRNA may be detected using methods the described in International Application Publication No. WO 2012/177906A1 and U.S. Patent Application No. US2014/0275211A1, which methods are incorporated herein by reference.

As used herein, “target sequence” refers to a contiguous portion of the nucleotide sequence of an mRNA molecule formed during the transcription of an HBV gene, including mRNA that is a product of RNA processing of a primary transcription product. The target portion of the sequence will be at least long enough to serve as a substrate for RNAi-directed cleavage at or near that portion. For example, the target sequence will generally be from 9-36 nucleotides in length, e.g., 15-30 nucleotides in length, including all sub-ranges there between. As non-limiting examples, a target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides,20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an siRNA as described herein include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, an siRNA comprising one oligonucleotide 21 nucleotides in length, and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary,” and “substantially complementary” herein can be used with respect to the base matching between the sense strand and the antisense strand of an siRNA, or between the antisense strand of an siRNA agent and a target sequence, as will be understood from the context of their use.

As used herein, a polynucleotide that is “substantially complementary” to at least part of a mRNA refers to a polynucleotide that is substantially complementary to a contiguous portion of the mRNA of interest (e.g., an mRNA encoding an HBV protein). For example, a polynucleotide is complementary to at least a part of an HBV mRNA if the sequence is substantially complementary to a non-interrupted portion of the HBV mRNA.

The term “siRNA,” as used herein, refers to an RNA interference molecule that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21 , 22, 23, 24, 25, 26, 27, 28, 29, 30, 31 , 32, 33, 34, 35, or 36 and any sub-range there between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, and 21-22 base pairs. siRNAs generated in the cell by processing with Dicer and similar enzymes are generally in the range of 19-22 base pairs in length.

One strand of the duplex region of an siRNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of an siRNA are comprised by separate RNA molecules, those molecules need not, but can be covalently connected. Where the two strands are connected covalently by means other than a hairpin loop, the connecting structure is referred to as a “linker.”

An siRNA as described herein can be synthesized by standard methods known in the art, e.g., by use of an automated DNA synthesizer, such as are commercially available from, for example, Biosearch, Applied Biosystems, Inc.

The term “antisense strand” or “guide strand” refers to the strand of an siRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

The term “sense strand” or “passenger strand” as used herein, refers to the strand of an siRNA that includes a region that is substantially complementary to a region of the antisense strand as that term is defined herein.

The term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure or in the ribose-phosphate backbone structure, e.g., as described in greater detail below. However, siRNA molecules comprising ribonucleoside analogs or derivatives retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate, or a non-natural base comprising nucleoside, or any combination thereof. In another example, an RNA molecule can comprise at least two modified ribonucleosides, at least 3, at least 4, at least 5, at least 6, at least 7, at least 8, at least 9, at least 10, at least 15, at least 20, or more, up to the entire length of the siRNA molecule. The modifications need not be the same for each of such a plurality of modified ribonucleosides in an RNA molecule. In some embodiments, a modified ribonucleoside includes a deoxyribonucleoside. For example, an siRNA can comprise one or more deoxynucleosides, including, for example, a deoxynucleoside overhang(s), or one or more deoxynucleosides within the double-stranded portion of an siRNA. However, the term “siRNA” as used herein does not include a fully DNA molecule.

As used herein, the term “nucleotide overhang” refers to at least one unpaired nucleotide that protrudes from the duplex structure of an siRNA. For example, when a 3′-end of one strand of an siRNA extends beyond the 5′-end of the other strand, or vice versa, there is a nucleotide overhang. An siRNA can comprise an overhang of at least one nucleotide; alternatively the overhang can comprise at least two nucleotides, at least three nucleotides, at least four nucleotides, at least five nucleotides, or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′ end, 3′ end, or both ends of either an antisense or sense strand of an siRNA.

The terms “blunt” or “blunt ended” as used herein in reference to an siRNA mean that there are no unpaired nucleotides or nucleotide analogs at a given terminal end of an siRNA, i.e., no nucleotide overhang. One or both ends of an siRNA can be blunt. Where both ends of an siRNA are blunt, the siRNA is said to be “blunt ended.” A “blunt ended” siRNA is an siRNA that is blunt at both ends, i.e., has no nucleotide overhang at either end of the molecule. Most often such a molecule will be double-stranded over its entire length.

II. siRNA Targeting HBV

The present disclosure provides methods of treatment involving administering an siRNA that targets HBV, and related compositions and kits. In some embodiments, the siRNA that targets HBV is HBV02. HBV02 is a synthetic, chemically modified siRNA targeting HBV RNA with a covalently attached triantennary N-acetyl-galactosamine (GalNAc) ligand that allows for specific uptake by hepatocytes. HBV02 targets a region of the HBV genome that is common to all HBV viral transcripts and is pharmacologically active against HBV genotypes A through J. In preclinical models, HBV02 has been shown to inhibit viral replication, translation, and secretion of HBsAg, and may provide a functional cure of chronic HBV infections. One siRNA can have multiple antiviral effects, including degradation of the pgRNA, thus inhibiting viral replication, and degradation of all viral mRNA transcripts, thereby preventing expression of viral proteins. This may result in the return of a functional immune response directed against HBV, either alone or in combination with other therapies. HBV02's ability to reduce HBsAg-containing noninfectious subviral particles also distinguishes it from currently available treatments.

HBV02 targets and inhibits expression of an mRNA encoded by an HBV genome according to NCBI Reference Sequence NC_003977.2 (GenBank Accession No. GI:21326584) (SEQ ID NO:1). More specifically, HBV02 targets an mRNA encoded by a portion of the HBV genome comprising the sequence GTGTGCACTTCGCTTCAC (SEQ ID NO:2), which corresponds to nucleotides 1579-1597 of SEQ ID NO:1. Because transcription of the HBV genome results in polycistronic, overlapping RNAs, HBV02 results in significant inhibition of expression of most or all HBV transcripts.

HBV02 has a sense strand comprising 5′-GUGUGCACUUCGCUUCACA-3′ (SEQ ID NO:3) and an antisense strand comprising 5′-UGUGAAGCGAAGUGCACACUU-3′ (SEQ ID NO:4) wherein the nucleotides include 2′-fluoro (2′F) and 2′-O-methoxy (2′OMe) ribose sugar modifications, phosphorothioate backbone modifications, a glycol nucleic acid (GNA) modification, and conjugation to a triantennary N-acetyl-galactosamine (GalNAc) ligand at the 3′ end of the sense strand, to facilitate delivery to hepatocytes through the asialoglycoprotein receptor (ASGPR). Including modifications, the sense strand of HBV02 comprises 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6), wherein the modifications are abbreviated as shown in Table 1.

TABLE 1
Abbreviations of nucleotide monomers used in modified nucleic
acid sequence representation. It will be understood that, unless
otherwise indicated, these monomers, when present in an oligonucleotide,
are mutually linked by 5′-3′-phosphodiester bonds.

| Abbreviation | Nucleotide(s) |
|---|---|
| A | adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | uridine-3′-phosphate |
| Uf | 2′-fluorouridine-3′-phosphate |
| Ufs | 2′-fluorouridine-3′-phosphorothioate |
| Us | uridine-3′-phosphorothioate |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | (Hyp-(GalNAc-alkyl)3) |
| (Agn) | adenosine-glycol nucleic acid (GNA) |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |

In some embodiments, the siRNA used in the methods, compositions, or kits described herein is HBV02.

In some embodiments, the siRNA used in the methods, compositions, or kits described herein comprises a sequence variant of HBV02. In particular embodiments, the portion of the HBV transcript(s) targeted by the sequence variant of HBV02 overlaps with the portion of the HBV transcript(s) targeted by HBV02.

In some embodiments, the siRNA comprises a sense strand and an antisense strand, wherein (1) the sense strand comprises SEQ ID NO:3 or SEQ ID NO:5, or a sequence that differs by not more than 4, not more than 3, not more than 2, or not more than 1 nucleotide from SEQ ID NO:3 or SEQ ID NO:5, respectively; or (2) the antisense strand comprises SEQ ID NO:4 or SEQ ID NO:6, or a sequence that differs by not more than 4, not more than 3, not more than 2, or not more than 1 nucleotide from SEQ ID NO:4 or SEQ ID NO:6, respectively.

In some embodiments, shorter duplexes having one of the sequences of SEQ ID NO:5 or SEQ ID NO:6 minus only a few nucleotides on one or both ends are used. Hence, siRNAs having a partial sequence of at least 15, 16, 17, 18, 19, 20, or more contiguous nucleotides from one or both of SEQ ID NO:5 and SEQ ID NO:6, and differing in their ability to inhibit the expression of an HBV gene by not more than 5, 10, 15, 20, 25, or 30% inhibition from an siRNA comprising the full sequence, are contemplated herein. In some embodiments, an siRNA having a blunt end at one or both ends, formed by removing nucleotides from one or both ends of HBV02, is provided.

In some embodiments, an siRNA as described herein can contain one or more mismatches to the target sequence. In some embodiments, an siRNA as described herein contains no more than 3 mismatches. In some embodiments, if the antisense strand of the siRNA contains mismatches to a target sequence, the area of mismatch is not located in the center of the region of complementarity. In particular embodiments, if the antisense strand contains mismatches to the target sequence, the mismatch is restricted to within the last 5 nucleotides from either the 5′ or 3′ end of the region of complementarity. For example, for a 23 nucleotide siRNA strand that is complementary to a region of an HBV gene, the RNA strand may not contain any mismatch within the central 13 nucleotides. The methods described herein or methods known in the art can be used to determine whether an siRNA containing a mismatch to a target sequence is effective in inhibiting the expression of an HBV gene.

In some embodiments, the siRNA used in the methods, compositions, and kits described herein include two oligonucleotides, where one oligonucleotide is described as the sense strand, and the second oligonucleotide is described as the corresponding antisense strand of the sense strand. As described elsewhere herein and as known in the art, the complementary sequences of an siRNA can also be contained as self-complementary regions of a single nucleic acid molecule, as opposed to being on separate oligonucleotides.

In some embodiments, a single-stranded antisense RNA molecule comprising the antisense strand of HBV02 or sequence variant thereof is used in the methods, compositions, and kits described herein. The antisense RNA molecule can have 15-30 nucleotides complementary to the target. For example, the antisense RNA molecule may have a sequence of at least 15, 16, 17, 18, 19, 20, 21, or more contiguous nucleotides from SEQ ID NO: 6.

In some embodiments, the siRNA comprises a sense strand and an antisense strand, wherein the sense strand comprises SEQ ID NO:5 and the antisense strand comprises SEQ ID NO:6, and further comprises additional nucleotides, modifications, or conjugates as described herein. For example, in some embodiments, the siRNA can include further modifications in addition to those indicated in SEQ ID NOs: 5 and 6. Such modifications can be generated using methods established in the art, such as those described in “Current protocols in nucleic acid chemistry,” Beaucage S L, et al. (Edrs.), John Wiley & Sons, Inc., New York, N.Y., USA, which methods are incorporated herein by reference. Examples of such modifications are described in more detail below.

a. Modified siRNAs

Modifications disclosed herein include, for example, (a) sugar modifications (e.g., at the 2′ position or 4′ position) or replacement of the sugar; (b) backbone modifications, including modification or replacement of the phosphodiester linkages; (c) base modifications, e.g., replacement with stabilizing bases, destabilizing bases, or bases that base pair with an expanded repertoire of partners, removal of bases (abasic nucleotides), or conjugated bases; and (d) end modifications, e.g., 5′ end modifications (phosphorylation, conjugation, inverted linkages, etc.), 3′ end modifications (conjugation, DNA nucleotides, inverted linkages, etc.). Some specific examples of modifications that can be incorporated into siRNAs of the present application are shown in Table 1.

Modifications include substituted sugar moieties. The siRNAs featured herein can include one of the following at the 2′ position: OH; F; O-, S—, or N-alkyl; O-, S—, or N-alkenyl; O-, S- or N-alkynyl; or O-alkyl-O-alkyl; wherein the alkyl, alkenyl, and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In some other embodiments, siRNAs include one of the following at the 2′ position: C₁ to Cm lower alkyl, substituted lower alkyl, alkaryl, aralkyl, O-alkaryl or O-aralkyl, SH, SCH₃, OCN, CI, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an siRNA, or a group for improving the pharmacodynamic properties of an siRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin, et al., Helv. Chim. Acta 1995, 78:486-504), i.e., an alkoxy-alkoxy group. Another exemplary modification is 2′-dimethylaminooxyethoxy, i.e., a O(CH₂)₂ON(CH₃)₂group, also known as 2′-DMAOE, and 2′-dimethylaminoethoxyethoxy (also known in the art as 2*-O-dimethylaminoethoxyethyl or 2*-DMAEOE), i.e., 2*-O—CH₂—O—CH₂—N(CH₂)₂. Other exemplary modifications include 2′-methoxy (2′-OCH₃), 2′-aminopropoxy (2-OCH₂CH₂CH₂NH₂), and 2′-fluoro (2′-F). Similar modifications can also be made at other positions on the RNA of an siRNA, particularly the 3′ position of the sugar on the 3′ terminal nucleotide or in 2′-5′ linked siRNAs and the 5′ position of the 5′ terminal nucleotide. Modifications can also include sugar mimetics, such as cyclobutyl moieties, in place of the pentofuranosyl sugar.

Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920; each of which is incorporated herein by reference for teachings relevant to methods of preparing such modifications.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6, 239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and RE39,464; each of which is herein incorporated herein by reference for teachings relevant to methods of preparing such modifications.

RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439; each of which is herein incorporated by reference for teachings relevant to methods of preparing such modifications.

In some embodiments, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262; each of which is incorporated herein by reference. Further teaching of PNA compounds can be found, for example, in Nielsen, et al. (Science, 254:1497-1500 (1991)).

Some embodiments featured in the technology described herein include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂—, and —N(CH₃)—CH₂—CH₂— [wherein the native phosphodiester backbone is represented as —O—P—O—CH₂—] of U.S. Pat. No. 5,489,677, and the amide backbones of U.S. Pat. No. 5,602,240. In some embodiments, the RNAs featured herein have morpholino backbone structures of U.S. Pat. No. 5,034,506.

Modifications of siRNAs disclosed herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl, and other 5-substituted uracils and cytosines, 7-methylguanine and 7-methyladenine, 8-azaguanine and 8-azaadenine, 7-deazaguanine and 7-daazaadenine, and 3-deazaguanine and 3-deazaadenine. Further nucleobases include those disclosed in U.S. Pat. No. 3,687,808, those disclosed in Modified Nucleosides in Biochemistry, Biotechnology and Medicine (Herdewijn P, ed., Wiley-VCH, 2008); those disclosed in The Concise Encyclopedia Of Polymer Science And Engineering (pages 858-859, Kroschwitz J L, ed., John Wiley & Sons, 1990), those disclosed by Englisch et al. (Angewandte Chemie, International Edition, 30, 613, 1991), and those disclosed by Sanghvi Y S (Chapter 15, dsRNA Research and Applications, pages 289-302, Crooke S T and Lebleu B, ed., CRC Press, 1993). Certain of these nucleobases are particularly useful for increasing the binding affinity of the oligomeric compounds featured in the technology described herein. These include 5-substituted pyrimidines, 6-azapyrimidines and N-2, N-6, and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi Y S, et al., Eds., dsRNA Research and Applications, CRC Press, Boca Raton, pp. 276-278, 1993) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, U.S. Pat. Nos. 3,687,808; 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088; each of which is incorporated herein by reference for teachings relevant to methods of preparing such modifications.

siRNAs can also be modified to include one or more adenosine-glycol nucleic acid (GNA). A description of adenosine-GNA can be found, for example, in Zhang, et al. (JACS 2005, 127(12):4174-75) which is incorporated herein by reference for teachings relevant to methods of preparing GNA modifications.

The RNA of an siRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen J, et al., Nucleic Acids Research 2005, 33(0:439-47; Mook O R, et al., Mol Cane Ther 2007, 6(3):833-43; Grunweller A, et al., Nucleic Acids Research 2003, 31(12):3185-93).

Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845; each of which is incorporated herein by reference for teachings relevant to methods of preparing such modifications.

In some embodiments, the siRNA includes modifications involving chemically linking to the RNA one or more ligands, moieties, or conjugates that enhance the activity, cellular distribution, or cellular uptake of the siRNA. Such moieties include but are not limited to lipid moieties such as a cholesterol moiety (Letsinger, et al., Proc. Natl. Acid. Sci. USA 1989, 86:6553-56), cholic acid (Manoharan, et al., Biorg. Med. Chem. Let. 1990, 4:1053-60), a thioether, e.g., beryl-S-tritylthiol (Manoharan, et al., Ann. N.Y. Acad. Sci. 1992, 660:306-9); Manoharan, et al., Biorg. Med. Chem. Let. 1993, 3:2765-70), a thiocholesterol (Oberhauser, et al., Nucl. Acids Res. 1992, 20:533-38), an aliphatic chain, e.g., dodecandiol or undecyl residues (Saison-Behmoaras, et al., EMBO J 1991, 10:1111-18; Kabanov, et al., FEBS Lett. 1990, 259:327-30; Svinarchuk, et al., Biochimie 1993, 75:49-54), a phospholipid, e.g., di-hexadecyl-rac-glycerol or triethyl-ammonium1,2-di-O-hexadecyl-rac-glycero-3-phosphonate (Manoharan, et al., Tetrahedron Lett. 1995, 36:3651-54; Shea, et al., Nucl. Acids Res. 1990, 18:3777-83), a polyamine or a polyethylene glycol chain (Manoharan, et al., Nucleosides & Nucleotides 1995, 14:969-73), or adamantane acetic acid (Manoharan, et al., Tetrahedron Lett. 1995, 36:3651-54), a palmityl moiety (Mishra, et al., Biochim. Biophys. Acta 1995, 1264:229-37), or an octadecylamine or hexylamino-carbonyloxycholesterol moiety (Crooke, et al., J. Pharmacol. Exp. Ther. 1996, 277:923-37).

In some embodiments, a ligand alters the distribution, targeting, or lifetime of an siRNA into which it is incorporated. In some embodiments, a ligand provides an enhanced affinity for a selected target, e.g., molecule, cell, or cell type, compartment, e.g., a cellular or organ compartment, tissue, organ, or region of the body, as, e.g., compared to a species absent such a ligand. In such embodiments, the ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Examples of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, and alpha helical peptide.

Ligands can also include targeting groups, e.g., a cell or tissue targeting agent, e.g., a lectin, glycoprotein, lipid or protein, e.g., an antibody, that binds to a specified cell type such as a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. Other examples of ligands include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules (e.g., cholesterol, cholic acid, adamantane acetic acid, 1-pyrene butyric acid, dihydrotestosterone, 1,3-Bis-0(hexadecyl)glycerol, geranyloxyhexyl group, hexadecylglycerol, borneol, menthol, 1,3-propanediol, heptadecyl group, palmitic acid, myristic acid,03-(oleoyl)lithocholic acid, 03-(oleoyl)cholenic acid, dimethoxytrityl, or phenoxazine), peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, and AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, and multivalent fucose. The ligand can be, for example, a lipopolysaccharide, an activator of p38 MAP kinase, or an activator of NF-KB.

The ligand can be a substance, e.g., a drug, which can increase the uptake of the siRNA into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, the ligand is a moiety, e.g., a vitamin, which is taken up by a target cell, e.g., a liver cell. Exemplary vitamins include vitamin A, E, and K. Other exemplary vitamins include are B vitamin, e.g., folic acid, B12, riboflavin, biotin, pyridoxal, or other vitamins or nutrients taken up by target cells such as liver cells. Also included are HSA and low density lipoprotein (LDL).

In some embodiments, a ligand attached to an siRNA as described herein acts as a pharmacokinetic (PK) modulator. As used herein, a “PK modulator” refers to a pharmacokinetic modulator. PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin, etc. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the technology described herein as ligands (e.g., as PK modulating ligands). In addition, aptamers that bind serum components (e.g., serum proteins) are also suitable for use as PK modulating ligands in the embodiments described herein.

(i) Lipid conjugates. In some embodiments, the ligand or conjugate is a lipid or lipid-based molecule. A lipid or lipid-based ligand can (a) increase resistance to degradation of the conjugate, (b) increase targeting or transport into a target cell or cell membrane, and/or (c) can be used to adjust binding to a serum protein, e.g., HSA. Such a lipid or lipid-based molecule may bind a serum protein, e.g., human serum albumin (HSA). An HSA-binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In some embodiments, the lipid based ligand binds HSA. The lipid based ligand may bind to HSA with a sufficient affinity such that the conjugate will be distributed to a non-kidney tissue. In certain particular embodiments, the HSA-ligand binding is reversible.

In some embodiments, the lipid based ligand binds HSA weakly or not at all, such that the conjugate will be distributed to the kidney. Other moieties that target to kidney cells can also be used in place of or in addition to the lipid based ligand.

(ii) Cell Permeation Peptide and Agents. In another aspect, the ligand is a cell-permeation agent, such as a helical cell-permeation agent. In some embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In some embodiments, the helical agent is an alpha-helical agent. In certain particular embodiments, the helical agent has a lipophilic and a lipophobic phase.

A “cell permeation peptide” is capable of permeating a cell, e.g., a microbial cell, such as a bacterial or fungal cell, or a mammalian cell, such as a human cell. A microbial cell-permeating peptide can be, for example, an alpha-helical linear peptide (e.g., LL-37 or Ceropin PI), a disulfide bond-containing peptide (e.g., a-defensin, β-defensin, or bactenecin), or a peptide containing only one or two dominating amino acids (e.g., PR-39 or indolicidin).

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to siRNA can affect pharmacokinetic distribution of the RNAi, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF, which has the amino acid sequence AAVALLPAVLLALLAP (SEQ ID NO:7). An RFGF analogue (e.g., amino acid sequence AALLPVLLAAP (SEQ ID NO:8) containing a hydrophobic MTS can also be a targeting moiety. The peptide moiety can be a “delivery” peptide, which can carry large polar molecules including peptides, oligonucleotides, and proteins across cell membranes. For example, sequences from the HIV Tat protein (GRKKRRQRRRPPQ (SEQ ID NO:9) and the Drosophila Antennapedia protein (RQIKIWFQNRRMKWK (SEQ ID NO:10) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam, et al., Nature 1991, 354:82-84).

A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni, et al., Nucl. Acids Res. 1993, 31:2717-24).

(iii) Carbohydrate Conjugates. In some embodiments, the siRNA oligonucleotides described herein further comprise carbohydrate conjugates. The carbohydrate conjugates may be advantageous for the in vivo delivery of nucleic acids, as well as compositions suitable for in vivo therapeutic use. As used herein, “carbohydrate” refers to a compound which is either a carbohydrate per se made up of one or more monosaccharide units having at least 6 carbon atoms (which can be linear, branched, or cyclic) with an oxygen, nitrogen, or sulfur atom bonded to each carbon atom; or a compound having as a part thereof a carbohydrate moiety made up of one or more monosaccharide units each having at least six carbon atoms (which can be linear, branched, or cyclic), with an oxygen, nitrogen, or sulfur atom bonded to each carbon atom. Representative carbohydrates include the sugars (mono-, di-, tri-, and oligosaccharides containing from about 4-9 monosaccharide units), and polysaccharides such as starches, glycogen, cellulose, and polysaccharide gums. Specific monosaccharides include C5 and above (in some embodiments, C5-C8) sugars; and di- and trisaccharides include sugars having two or three monosaccharide units (in some embodiments, C5-C8).

In some embodiments, the carbohydrate conjugate is selected from the group consisting of:

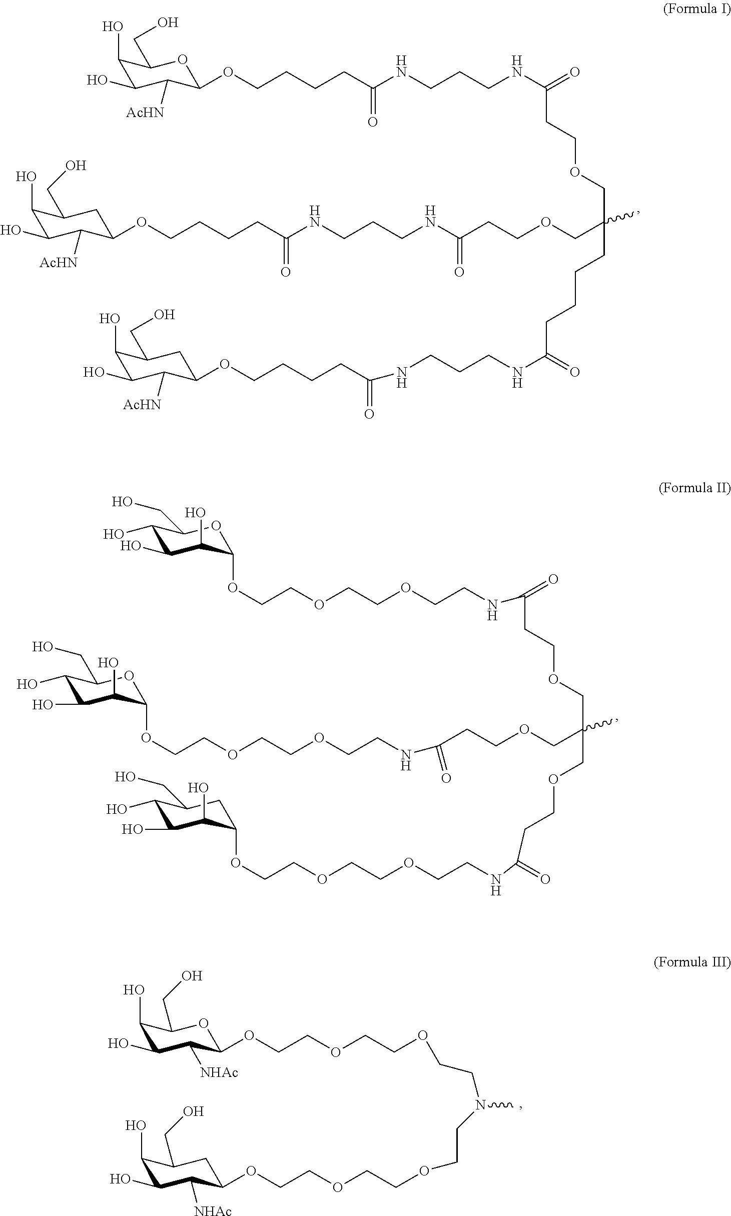

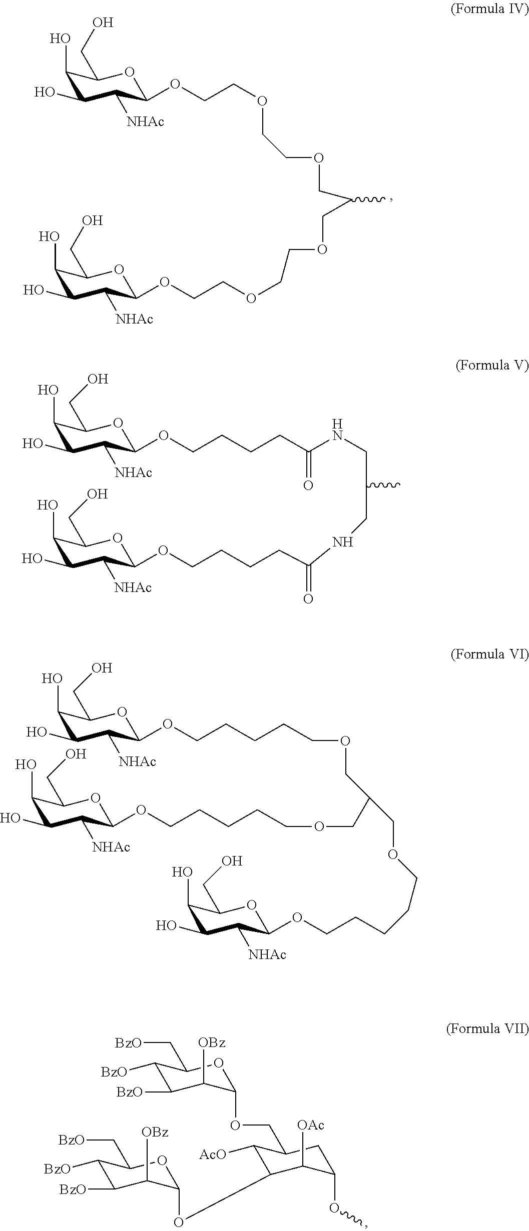

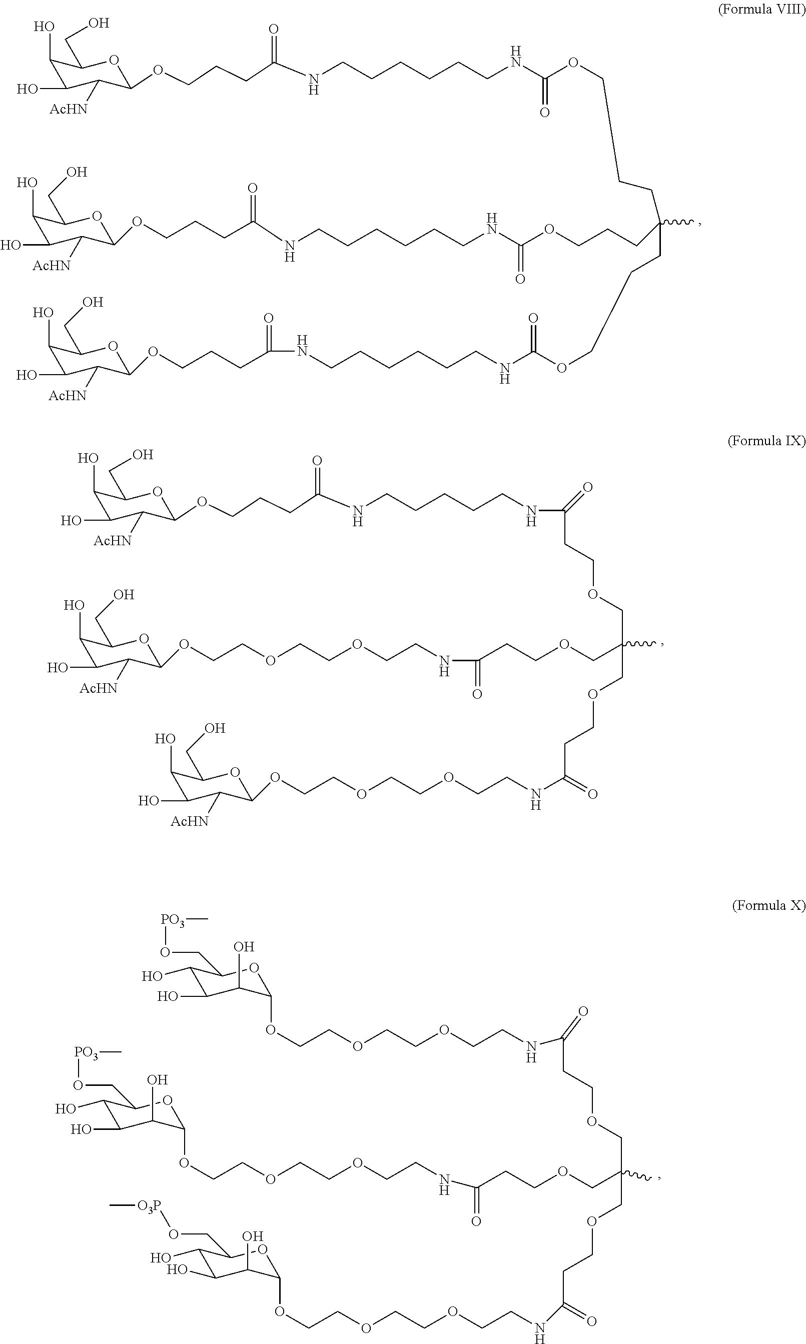

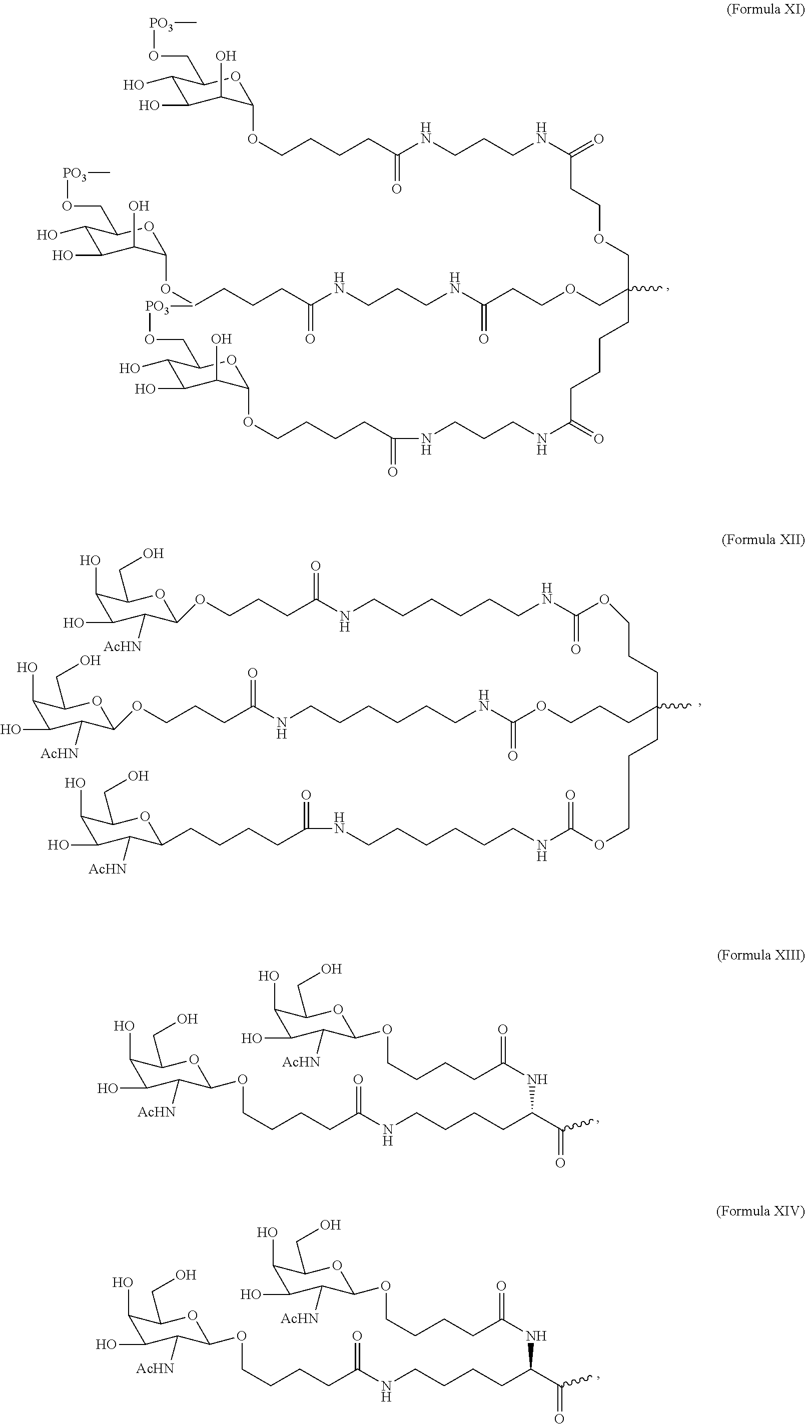

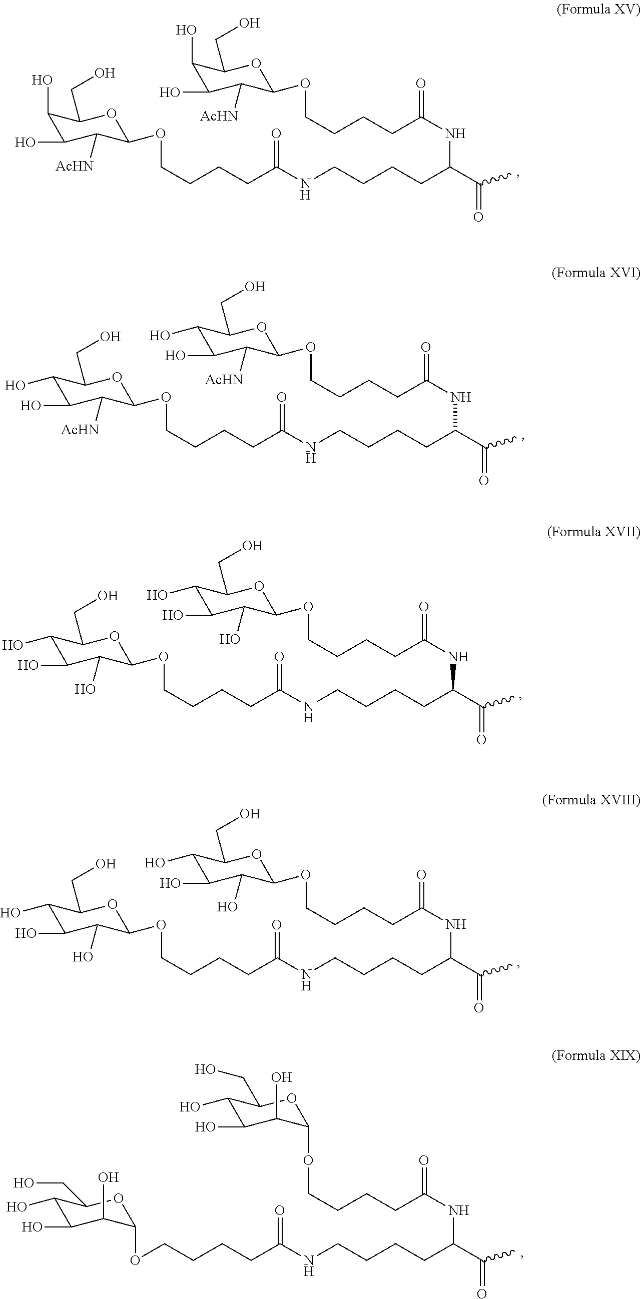

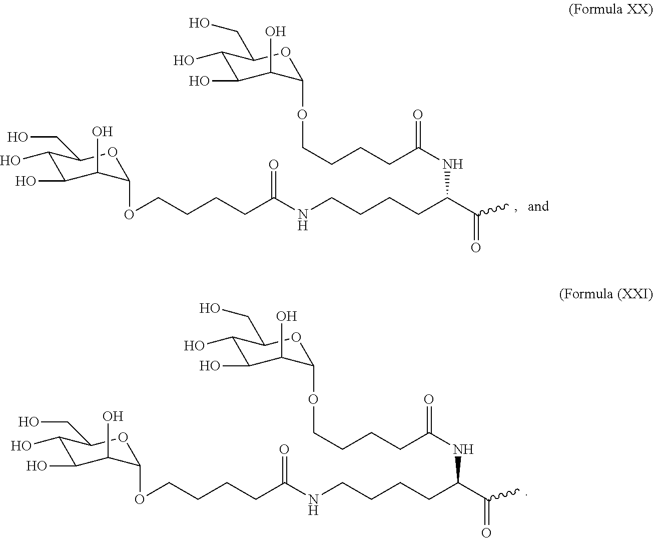

Another representative carbohydrate conjugate for use in the embodiments described herein includes, but is not limited to,

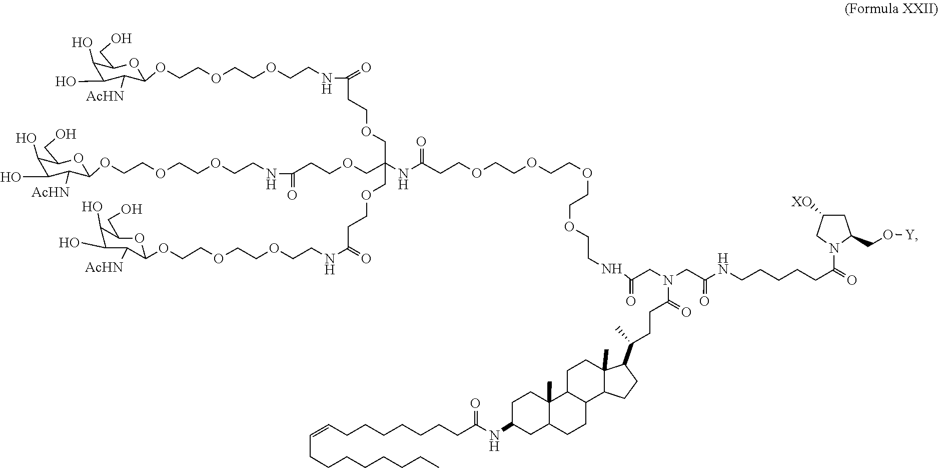

wherein when one of X or Y is an oligonucleotide, the other is a hydrogen.

In some embodiments, the carbohydrate conjugate further comprises another ligand such as, but not limited to, a PK modulator, an endosomolytic ligand, or a cell permeation peptide.

(iv) Linkers. In some embodiments, the conjugates described herein can be attached to the siRNA oligonucleotide with various linkers that can be cleavable or non-cleavable.

The term “linker” or “linking group” means an organic moiety that connects two parts of a compound. Linkers typically comprise a direct bond or an atom such as oxygen or sulfur, a unit such as NR8, C(O), C(O)NH, SO, SO2, SO2NH, or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, and alkynylhereroaryl, which one or more methylenes can be interrupted or terminated by O, S, S(O), SO2, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In certain embodiments, the linker is between 1-24 atoms, between 4-24 atoms, between 6-18 atoms, between 8-18 atoms, or between 8-16 atoms.

A cleavable linking group is one which is sufficiently stable outside the cell, but which upon entry into a target cell is cleaved to release the two parts the linker is holding together. In certain embodiments, the cleavable linking group is cleaved at least 10 times, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases. A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a particular pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver-targeting ligands can be linked to the cationic lipids through a linker that includes an ester group. Liver cells are rich in esterases, and therefore the linker will be cleaved more efficiently in liver cells than in cell types that are not esterase-rich. Other cell types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It can be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell-free systems, in cells, in cell culture, in organ or tissue culture, or in whole animals. It can be useful to make initial evaluations in cell-free or culture conditions and to confirm by further evaluations in whole animals. In certain embodiments, useful candidate compounds are cleaved at least 2, at least 4, at least 10 or at least 100 times faster in the cell (or under in vitro conditions selected to mimic intracellular conditions) as compared to blood or serum (or under in vitro conditions selected to mimic extracellular conditions).

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular RNAi moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In some embodiments, candidate compounds are cleaved by at most 10% in the blood. In certain embodiments, useful candidate compounds are degraded at least 2, at least 4, at least 10, or at least 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. In certain embodiments, the phosphate-based linking groups are selected from: —O—P(O)(OH)—O—, —O—P(S)(OH)—O—, —O—P(S)(SH)—O—, —S—P(O)(OH)—O—, —O—P(0)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—. In particular embodiments, the phosphate-linking group is —O—P(O)(OH)—O—. These candidates can be evaluated using methods analogous to those described above.

Acid cleavable linking groups are linking groups that are cleaved under acidic conditions. In some embodiments, acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes, can provide a cleaving environment for acid cleavable linking groups. Examples of acid cleavable linking groups include but are not limited to hydrazones, esters, and esters of amino acids. Acid cleavable groups can have the general formula —C═N—, C(O)O, or —OC(O). In some embodiments, the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups are cleaved by enzymes such as esterases and amidases in cells. Examples of ester-based cleavable linking groups include but are not limited to esters of alkylene, alkenylene, and alkynylene groups. Ester cleavable linking groups have the general formula —C(O)O—, or —OC(O)—. These candidates can be evaluated using methods analogous to those described above.

Peptide-based cleavable linking groups are cleaved by enzymes such as peptidases and proteases in cells. Peptide-based cleavable linking groups are peptide bonds formed between amino acids to yield oligopeptides (e.g., dipeptides, tripeptides, etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

Representative carbohydrate conjugates with linkers include, but are not limited to,

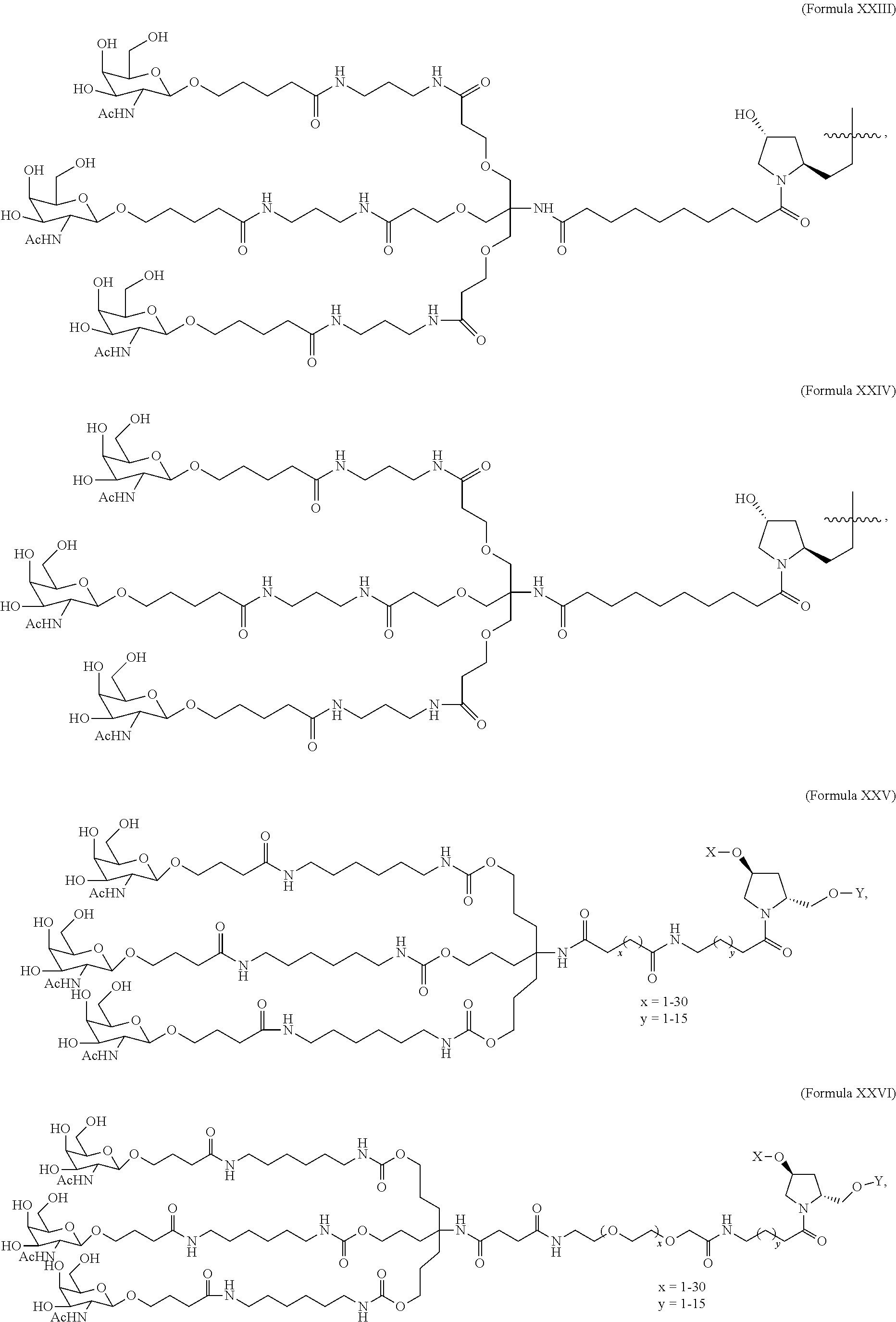

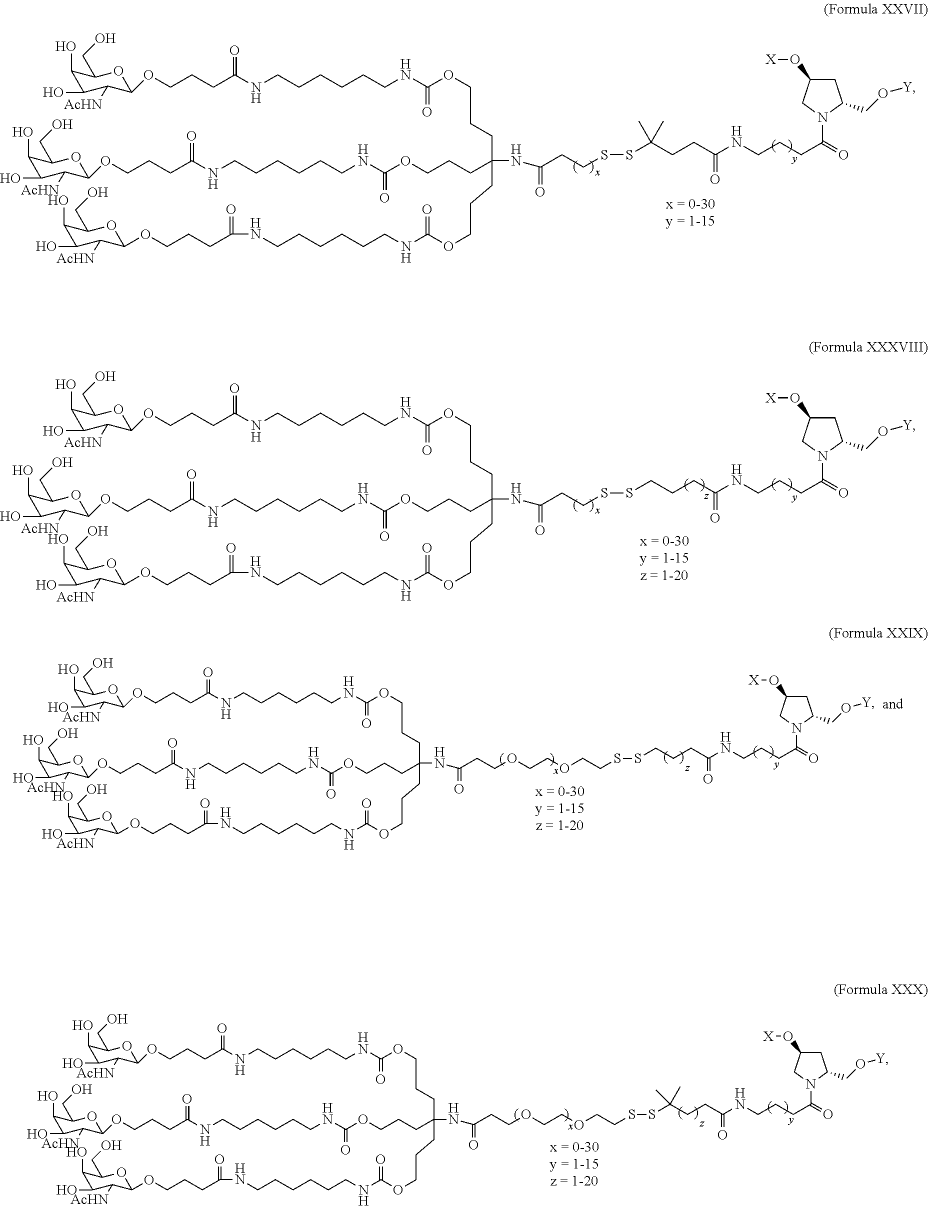

wherein when one of X or Y is an oligonucleotide, the other is a hydrogen.

In certain embodiments of the compositions and methods, a ligand is one or more “GalNAc” (N-acetylgalactosamine) derivatives attached through a bivalent or trivalent branched linker. For example, in some embodiments the siRNA is conjugated to a GalNAc ligand as shown in the following schematic:

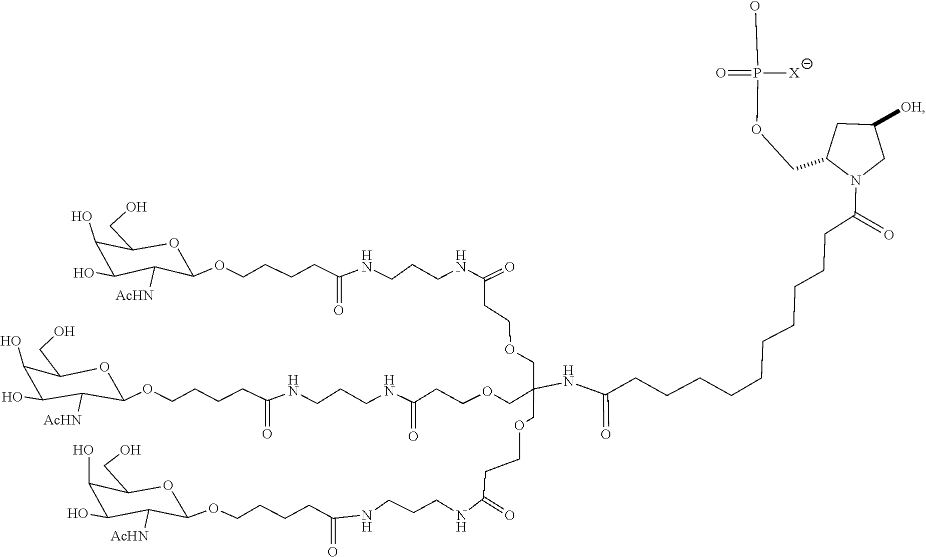

wherein X is O or S.

In some embodiments, the combination therapy includes an siRNA that is conjugated to a bivalent or trivalent branched linker selected from the group of structures shown in any of formula (XXXI)-(XXXIV):

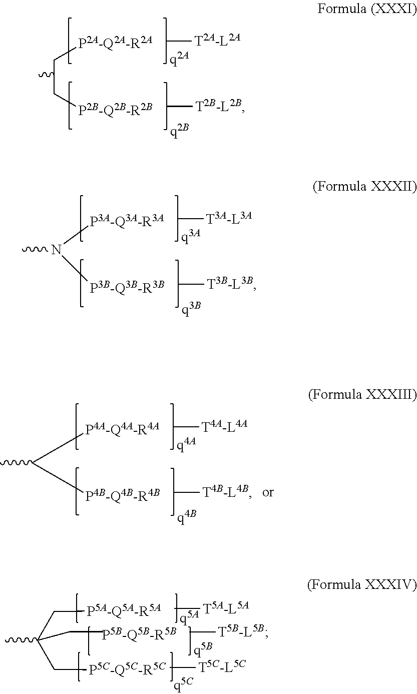

wherein:

q2A, q2B, q3A, q3B, q4A, q4B, q5A, q5B, and q5C represent independently for each occurrence 0-20 and wherein the repeating unit can be the same or different;

P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, and T^{5C} are each independently for each occurrence absent, CO, NH, O, S, OC(O), NHC(O), CH₂, CH₂NH, or CH₂O;

Q^{2A}, Q^{2B}, Q^{3A}, Q^{3B}, Q^{4A}, Q^{4B}, Q^{5A}, Q^{5B}, and Q^{5C} are independently for each occurrence absent, alkylene, or substituted alkylene wherein one or more methylenes can be interrupted or terminated by one or more of O, S, S(O), SO₂, N(R^{N}), C(R′)═C(R″), C≡C or C(O);

R^{2A}, R^{2B}, R^{3A}, R^{3B}, R^{4A}, R^{4B}, R^{5A}, R^{5B}, and R^{5C} are each independently for each occurrence absent, NH, O, S, CH₂, C(O)O, C(O)NH, NHCH(R^{a})C(O), —C(O)—CH(R^{a})—NH—, CO, CH═N—O,

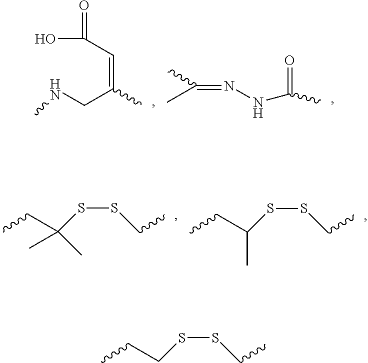

or heterocyclyl;

L^{2A}, L^{2B}, L^{3A}, L^{3B}, L^{4A}, L^{4B}, L^{5A}, L^{5B}, and L^{5C} represent the ligand; i.e., each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with siRNAs for inhibiting the expression of a target gene, such as those of formula (XXXV):

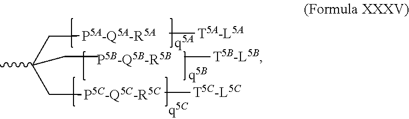

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas I, VI, X, IX, and XII.

Representative U.S. patents that teach the preparation of RNA conjugates include U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; and 7,037,646; each of which is incorporated herein by reference for the teachings relevant to such methods of preparation.

In certain instances, the RNA of an siRNA can be modified by a non-ligand group. A number of non-ligand molecules have been conjugated to siRNAs in order to enhance the activity, cellular distribution or cellular uptake of the siRNAs, and procedures for performing such conjugations are available in the scientific literature. Such non-ligand moieties have included lipid moieties, such as cholesterol (Kubo, T., et al., Biochem. Biophys. Res. Comm. 365(1):54-61 (2007); Letsinger, et al., Proc. Natl. Acad. Sci. USA 86:6553 (1989)), cholic acid (Manoharan, et al., Bioorg. Med. Chem. Lett. 4:1053 (1994)), a thioether, e.g., hexyl-S-tritylthiol (Manoharan, et al., Ann. N.Y. Acad. Sci. 660:306 (1992); Manoharan, et al., Bioorg. Med. Chem. Let. 3:2765 (1993)), a thiocholesterol (Oberhauser, et al., Nucl. Acids Res. 20:533 (1992)), an aliphatic chain, e.g., dodecandiol or undecyl residues (Saison-Behmoaras, et al., EMBO J. 10:111 (1991); Kabanov, et al., FEBS Lett. 259:327 (1990); Svinarchuk, et al., Biochimie 75:49 (1993)), a phospholipid, e.g., di-hexadecyl-rac-glycerol or triethylammonium1,2-di-O-hexadecyl-rac-glycero-3-H-phosphonate (Manoharan, et al., Tetrahedron Lett. 36:3651 (1995); Shea, et al., Nucl. Acids Res. 18:3777 (1990)), a polyamine or a polyethylene glycol chain (Manoharan, et al., Nucleosides & Nucleotides 14:969 (1995)), or adamantane acetic acid (Manoharan, et al., Tetrahedron Lett. 36:3651 (1195)), a palmityl moiety (Mishra, et al., Biochim. Biophys. Acta 1264:229 (1995)), or an octadecylamine or hexylamino-carbonyl-oxycholesterol moiety (Crooke, et al., J. Pharmacol. Exp. Ther. 277:923 (1996)).

Typical conjugation protocols involve the synthesis of an RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

b. Pharmaceutical Compositions and Delivery of siRNA

In some embodiments, pharmaceutical compositions containing an siRNA, as described herein, and a pharmaceutically acceptable carrier or excipient are provided. The pharmaceutical composition containing the siRNA can be used to treat HBV infection. Such pharmaceutical compositions are formulated based on the mode of delivery. For example, compositions may be formulated for systemic administration via parenteral delivery, e.g., by subcutaneous (SC) delivery.

A “pharmaceutically acceptable carrier” or “excipient” is a pharmaceutically acceptable solvent, suspending agent, or any other pharmacologically inert vehicle for delivering one or more agents, such as nucleic acids, to an animal. The excipient can be liquid or solid and is selected, with the planned manner of administration in mind, so as to provide for the desired bulk, consistency, etc., when combined with the agent (e.g., a nucleic acid) and the other components of a given pharmaceutical composition. Typical pharmaceutically acceptable carriers or excipients include, but are not limited to, binding agents (e.g., pregelatinized maize starch, polyvinylpyrrolidone, hydroxypropyl methylcellulose); fillers (e.g., lactose and other sugars, microcrystalline cellulose, pectin, gelatin, calcium sulfate, ethyl cellulose, polyacrylates, calcium hydrogen phosphate); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate); disintegrants (e.g., starch, sodium starch glycolate); and wetting agents (e.g., sodium lauryl sulphate).

Pharmaceutically acceptable organic or inorganic excipients suitable for non-parenteral administration that do not deleteriously react with nucleic acids can also be used to formulate siRNA compositions. Suitable pharmaceutically acceptable carriers for formulations used in non-parenteral delivery include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents, and other suitable additives. Pharmaceutically acceptable organic or inorganic excipients suitable for non-parenteral administration that do not deleteriously react with nucleic acids can be used.

In some embodiments, administration of pharmaceutical compositions and formulations described herein can be topical (e.g., by a transdermal patch), pulmonary (e.g., by inhalation or insufflation of powders or aerosols, including by nebulizer); intratracheal; intranasal; epidermal and transdermal; oral; or parenteral. Parenteral administration includes intravenous, intraarterial, subcutaneous, intraperitoneal, and intramuscular injection or infusion; subdermal administration (e.g., via an implanted device); or intracranial administration (e.g., by intraparenchymal, intrathecal, or intraventricular, administration).

In some embodiments, the pharmaceutical composition comprises a sterile solution of HBV02 formulated in water for subcutaneous injection. In some embodiments, the pharmaceutical composition comprises a sterile solution of HBV02 formulated in water for subcutaneous injection at a free acid concentration of 200 mg/mL.

In some embodiments, the pharmaceutical compositions containing an siRNA described herein are administered in dosages sufficient to inhibit expression of an HBV gene. In some embodiments, a dose of an siRNA is in the range of 0.001 to 200.0 milligrams per kilogram body weight of the recipient per day, or in the range of 1 to 50 milligrams per kilogram body weight per day. For example, an siRNA can be administered at 0.01 mg/kg, 0.05 mg/kg, 0.5 mg/kg, 1 mg/kg, 1.5 mg/kg, 2 mg/kg, 3 mg/kg, 10 mg/kg, 20 mg/kg, 30 mg/kg, 40 mg/kg, or 50 mg/kg per single dose. The pharmaceutical composition can be administered once daily, or it can be administered as two, three, or more sub-doses at appropriate intervals throughout the day or even using continuous infusion or delivery through a controlled release formulation. In that case, the siRNA contained in each sub-dose must be correspondingly smaller in order to achieve the total daily dosage. The dosage unit can also be compounded for delivery over several days, e.g., using a conventional sustained release formulation which provides sustained release of the siRNA over a several day period. Sustained release formulations are well known in the art and are particularly useful for delivery of agents at a particular site, such as could be used with the agents of the technology described herein. In such embodiments, the dosage unit contains a corresponding multiple of the daily dose.

In some embodiments, a pharmaceutical composition comprising an siRNA that targets HBV described herein (e.g., HBV02) contains the siRNA at a dose of 0.8 mg/kg, 1.7 mg/kg, 3.3 mg/kg, 6.7 mg/kg, or 15 mg/kg.

In some embodiments, a pharmaceutical composition comprising an siRNA described herein (e.g., HBV02) contains the siRNA at a dose of 20 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg.

In some embodiments, a pharmaceutical composition comprising an siRNA described herein (e.g., HBV02) contains the siRNA at a dose of 20 mg, 50 mg, 100 mg, 150 mg, 200 mg, 250 mg, 300 mg, 400 mg, or 450 mg.

In some embodiments, a pharmaceutical composition comprising an siRNA described herein (e.g., HBV02) contains the siRNA at a dose of 200 mg.

### III. Methods of Treatment and Additional Therapeutic Agents

The present disclosure provides for methods of treating HBV infection with an siRNA described herein. In some embodiments, a method of treating HBV comprising administering HBV02 to the subject is provided.

In some embodiments of the aforementioned methods, the method further comprises administering pegylated interferon-alpha (PEG-IFNα) to the subject.

In some further embodiments of the aforementioned methods, the method further comprises administering a nucleoside/nucleotide reverse transcriptase inhibitor (NRTI) to the subject. In some embodiments, the NRTI is administered before, simultaneously with, or sequentially after administration of the HBV02.

In some embodiments, a method of treating HBV is provided, comprising administering HBV02, and PEG-IFNα to a subject. In some embodiments, the PEG-IFNα is administered before, simultaneously with, or sequentially after administration of the HBV02.

In some embodiments, a method of treating HBV is provided, comprising administering HBV02, and PEG-IFNα, to a subject, wherein the subject has previously been administered an NRTI. In some embodiments, the PEG-IFNα is simultaneously with, or sequentially after administration of the HBV02.

In some embodiments, a method of treating HBV is provided, comprising administering HBV02, wherein the subject has previously been administered PEG-IFNα and previously administered an NRTI.

In any of the aforementioned methods, the HBV infection may be chronic HBV infection.

As used herein, “nucleoside/nucleotide reverse transcriptase inhibitor” or “nucleos(t)ide reverse transcriptase inhibitor” (NRTI) refers to an inhibitor of DNA replication that is structurally similar to a nucleotide or nucleoside and specifically inhibits replication of the HBV cccDNA by inhibiting the action of HBV polymerase, and does not significantly inhibit the replication of the host (e.g., human) DNA. Such inhibitors include tenofovir, tenofovir disoproxil fumarate (TDF), tenofovir alafenamide (TAF), lamivudine, adefovir, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, ganciclovir, besifovir (ANA-380/LB-80380), and tenofvir-exaliades (TLX/CMX157). In some embodiments, the NRTI is entecavir (ETV). In some embodiments, the NRTI is tenofovir. In some embodiments, the NRTI is lamivudine. In some embodiments, the NRTI is adefovir or adefovir dipivoxil.

As used herein, a “subject” is an animal, such as a mammal, including any mammal that can be infected with HBV, e.g., a primate (such as a human, a non-human primate, e.g., a monkey, or a chimpanzee), or an animal that is considered an acceptable clinical model of HBV infection, HBV-AAV mouse model (see, e.g., Yang, et al., Cell and Mol Immunol 11:71 (2014)) or the HBV 1.3xfs transgenic mouse model (Guidotti, et al., J. Virol. 69:6158 (1995)). In some embodiments, the subject has a hepatitis B virus (HBV) infection. In some embodiments, the subject is a human, such as a human being having an HBV infection, especially a chronic hepatitis B virus infection.

As used herein, the terms “treating” or “treatment” refer to a beneficial or desired result including, but not limited to, alleviation or amelioration of one or more signs or symptoms associated with unwanted HBV gene expression or HBV replication, e.g., the presence of serum or liver HBV cccDNA, the presence of serum HBV DNA, the presence of serum or liver HBV antigen, e.g., HBsAg or HBeAg, elevated ALT, elevated AST (normal range is typically considered about 10 to 34 U/L), the absence of or low level of anti-HBV antibodies; a liver injury; cirrhosis; delta hepatitis; acute hepatitis B; acute fulminant hepatitis B; chronic hepatitis B; liver fibrosis; end-stage liver disease; hepatocellular carcinoma; serum sickness-like syndrome; anorexia; nausea; vomiting, low-grade fever; myalgia; fatigability; disordered gustatory acuity and smell sensations (aversion to food and cigarettes); or right upper quadrant and epigastric pain (intermittent, mild to moderate); hepatic encephalopathy; somnolence; disturbances in sleep pattern; mental confusion; coma; ascites; gastrointestinal bleeding; coagulopathy; jaundice; hepatomegaly (mildly enlarged, soft liver); splenomegaly; palmar erythema; spider nevi; muscle wasting; spider angiomas; vasculitis; variceal bleeding; peripheral edema; gynecomastia; testicular atrophy; abdominal collateral veins (caput medusa); ALT levels higher than AST levels; elevated gamma-glutamyl transpeptidase (GGT) (normal range is typically considered about 8 to 65 U/L) and alkaline phosphatase (ALP) levels (normal range is typically considered about 44 to 147 IU/L (international units per liter), not more than 3 times the ULN); slightly low albumin levels; elevated serum iron levels; leukopenia (i.e., granulocytopenia); lymphocytosis; increased erythrocyte sedimentation rate (ESR); shortened red blood cell survival; hemolysis; thrombocytopenia; a prolongation of the international normalized ratio (INR); presence of serum or liver HBsAg, HBeAg, Hepatitis B core antibody (anti-HBc) immunoglobulin M (IgM); hepatitis B surface antibody (anti-HBs), hepatitis B e antibody (anti-HBe), or HBV DNA; increased bilirubin levels; hyperglobulinemia; the presence of tissue-nonspecific antibodies, such as anti-smooth muscle antibodies (ASMAs) or antinuclear antibodies (ANAs) (10-20%); the presence of tissue-specific antibodies, such as antibodies against the thyroid gland (10-20%); elevated levels of rheumatoid factor (RF); low platelet and white blood cell counts; lobular, with degenerative and regenerative hepatocellular changes, and accompanying inflammation; and predominantly centrilobular necrosis, whether detectable or undetectable. The likelihood of developing, e.g., liver fibrosis, is reduced, for example, when an individual having one or more risk factors for liver fibrosis, e.g., chronic hepatitis B infection, either fails to develop liver fibrosis or develops liver fibrosis with less severity relative to a population having the same risk factors and not receiving treatment as described herein. “Treatment” can also mean prolonging survival as compared to expected survival in the absence of treatment.

As used herein, the terms “preventing” or “prevention” refer to the failure to develop a disease, disorder, or condition, or the reduction in the development of a sign or symptom associated with such a disease, disorder, or condition (e.g., by a clinically relevant amount), or the exhibition of delayed signs or symptoms delayed (e.g., by days, weeks, months, or years). Prevention may require the administration of more than one dose.

In some embodiments, treatment of HBV infection results in a “functional cure” of hepatitis B. As used herein, functional cure is understood as clearance of circulating HBsAg and is may be accompanied by conversion to a status in which HBsAg antibodies become detectable using a clinically relevant assay. For example, detectable antibodies can include a signal higher than 10 mIU/ml as measured by Chemiluminescent Microparticle Immunoassay (CMIA) or any other immunoassay. Functional cure does not require clearance of all replicative forms of HBV (e.g., cccDNA from the liver). Anti-HBs seroconversion occurs spontaneously in about 0.2-1% of chronically infected patients per year. However, even after anti-HBs seroconversion, low level persistence of HBV is often observed for decades indicating that a functional rather than a complete cure occurs. Without being bound to a particular mechanism, the immune system may be able to keep HBV in check under conditions in which a functional cure has been achieved. A functional cure permits discontinuation of any treatment for the HBV infection. However, it is understood that a “functional cure” for HBV infection may not be sufficient to prevent or treat diseases or conditions that result from HBV infection, e.g., liver fibrosis, HCC, or cirrhosis. In some specific embodiments, a “functional cure” can refer to a sustained reduction in serum HBsAg, such as <1 IU/mL, for at least 3 months, at least 6 months, or at least one year following the initiation of a treatment regimen or the completion of a treatment regimen. The formal endpoint accepted by the U.S. Food and Drug Administration, or the FDA, for demonstrating a functional cure of HBV is undetectable HBsAg, defined as less than 0.05 international units per milliliter, or IU/ml, as well as HBV DNA less than the lower limit of quantification, in the blood six months after the end of therapy.

As used herein, the term “Hepatitis B virus-associated disease” or “HBV-associated disease,” is a disease or disorder that is caused by, or associated with HBV infection or replication. The term “HBV-associated disease” includes a disease, disorder or condition that would benefit from reduction in HBV gene expression or replication. Non-limiting examples of HBV-associated diseases include, for example, hepatitis D virus infection, delta hepatitis, acute hepatitis B; acute fulminant hepatitis B; chronic hepatitis B; liver fibrosis; end-stage liver disease; and hepatocellular carcinoma.

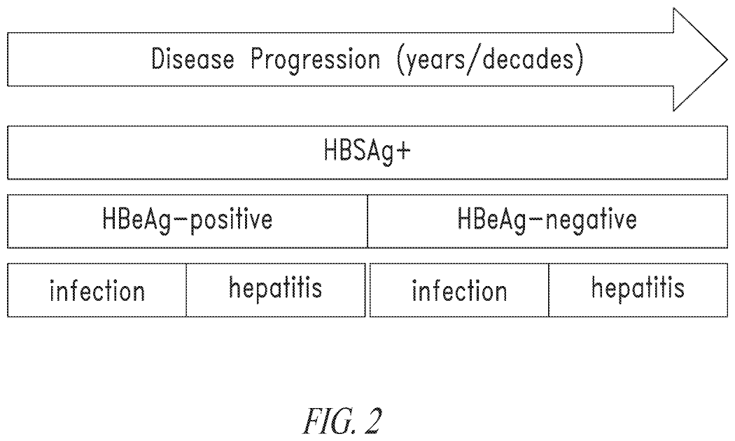

In some embodiments, an HBV-associated disease is chronic hepatitis. Chronic hepatitis B is defined by one of the following criteria: (1) positive serum HBsAg, HBV DNA, or HBeAg on two occasions at least 6 months apart (any combination of these tests performed 6 months apart is acceptable); or (2) negative immunoglobulin M (IgM) antibodies to HBV core antigen (IgM anti-HBc) and a positive result on one of the following tests: HBsAg, HBeAg, or HBV DNA (see FIG. 2). Chronic HBV typically includes inflammation of the liver that lasts more than six months. Subjects having chronic HBV are HBsAg positive and have either high viremia (≥10⁴ HBV-DNA copies/ml blood) or low viremia (<10³ HBV-DNA copies/ml blood). In certain embodiments, subjects have been infected with HBV for at least five years. In certain embodiments, subjects have been infected with HBV for at least ten years. In certain embodiments, subjects became infected with HBV at birth. Subjects having chronic hepatitis B disease can be immune tolerant or have an inactive chronic infection without any evidence of active disease, and they are also asymptomatic. Patients with chronic active hepatitis, especially during the replicative state, may have symptoms similar to those of acute hepatitis. Subjects having chronic hepatitis B disease may have an active chronic infection accompanied by necroinflammatory liver disease, have increased hepatocyte turn-over in the absence of detectable necroinflammation, or have an inactive chronic infection without any evidence of active disease, and they are also asymptomatic. The persistence of HBV infection in chronic HBV subjects is the result of cccHBV DNA.

HBeAg status represents multiple differences between subjects (Table 2). HBeAg status may affect responses to different therapies, and approximately one third of patients with HBV are HBeAg-positive.

TABLE 2
HBeAg status.

| | HBeAg-positive | HBeAg-negative |
|---|---|---|
| Age | Younger | Older |
| Approximate average | 10⁴ − 10⁵ IU/mL | 10³ IU/mL |
| HBsAg levels | | |
| Transcriptional activity | cccDNA > intDNA | intDNA > cccDNA |
| HBV-specific immune | Less compromised | More compromised |
| profile | | |

In some embodiments, a subject having chronic HBV is HBeAg positive. In some other embodiments, a subject having chronic HBV is HBeAg negative. Subjects having chronic HBV have a level of serum HBV DNA of less than 10⁵ and a persistent elevation in transaminases, for examples ALT, AST, and gamma-glutamyl transferase. A subject having chronic HBV may have a liver biopsy score of less than 4 (e.g., a necroinflammatory score).

In some embodiments, an HBV-associated disease is acute fulminant hepatitis B. A subject having acute fulminant hepatitis B has symptoms of acute hepatitis and the additional symptoms of confusion or coma (due to the liver's failure to detoxify chemicals) and bruising or bleeding (due to a lack of blood clotting factors).

Subjects having an HBV infection, e.g., chronic HBV, may develop liver fibrosis. Accordingly, in some embodiments, an HBV-associated disease is liver fibrosis. Liver fibrosis, or cirrhosis, is defined histologically as a diffuse hepatic process characterized by fibrosis (excess fibrous connective tissue) and the conversion of normal liver architecture into structurally abnormal nodules.

Subjects having an HBV infection, e.g., chronic HBV, may develop end-stage liver disease. Accordingly, in some embodiments, an HBV-associated disease is end-stage liver disease. For example, liver fibrosis may progress to a point where the body may no longer be able to compensate for, e.g., reduced liver function, as a result of liver fibrosis (i.e., decompensated liver), and result in, e.g., mental and neurological symptoms and liver failure.

Subjects having an HBV infection, e.g., chronic HBV, may develop hepatocellular carcinoma (HCC), also referred to as malignant hepatoma. Accordingly, in some embodiments, an HBV-associated disease is HCC. HCC commonly develops in subjects having chronic HBV and may be fibrolamellar, pseudoglandular (adenoid), pleomorphic (giant cell), or clear cell.

In some embodiments of the methods and uses described herein, a therapeutically effective amount of siRNA, PEG-IFNα, or both is administered to a subject. “Therapeutically effective amount,” as used herein, is intended to include the amount of an active agent, that, when administered to a subject for treating a subject having an HBV infection or HBV-associated disease, is sufficient to effect treatment of the disease (e.g., by diminishing or maintaining the existing disease or one or more symptoms of disease). The “therapeutically effective amount” may vary depending on the active agent, how it is administered, the disease and its severity, and the history, age, weight, family history, genetic makeup, stage of pathological processes mediated by HBV gene expression, the types of preceding or concomitant treatments, if any, and other individual characteristics of the subject to be treated. A therapeutically effective amount may require the administration of more than one dose.

A “therapeutically effective amount” also includes an amount of an active agent that produces some desired effect at a reasonable benefit/risk ratio applicable to any treatment. Therapeutic agents (e.g. siRNA, PEG-IFNα) used in the methods of the present disclosure may be administered in a sufficient amount to produce a reasonable benefit/risk ratio applicable to such treatment.

The term “sample,” as used herein, includes a collection of similar fluids, cells, or tissues isolated from a subject, as well as fluids, cells, or tissues present within a subject. Examples of biological fluids include blood, serum, and serosal fluids, plasma, lymph, urine, saliva, and the like. Tissue samples may include samples from tissues, organs or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In certain embodiments, a “sample derived from a subject” refers to blood, or plasma or serum obtained from blood drawn from the subject. In further embodiments, a “sample derived from a subject” refers to liver tissue (or subcomponents thereof) or blood tissue (or subcomponents thereof, e.g., serum) derived from the subject.

Some embodiments of the present disclosure provide methods of treating chronic HBV infection or an HBV-associated disease in a subject in need thereof, comprising: administering to the subject an siRNA, wherein the siRNA has a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Agn) is adenosine-glycol nucleic acid (GNA); s is a phosphorothioate linkage; and L96 is N-[tris(GalNAc-alkyl)-amidodecanoyl)]-4-hydroxyprolinol. In some embodiments of the methods, the method further comprises administering to the subject a peglyated interferon-alpha (PEG-IFNα). In some embodiments, the siRNA and PEG-IFNα are administered to the subject over the same time period. In some embodiments, siRNA is administered to the subject for a period of time before the PEG-IFNα is administered to the subject. In some embodiments, the PEG-IFNα is administered to the subject for a period of time before the siRNA is administered to the subject. In some embodiments, the subject has been administered PEG-IFNα prior to the administration of the siRNA. In some embodiments, the subject is administered PEG-IFNα during the same period of time that the subject is administered the siRNA. In some embodiments, the subject is subsequently administered PEG-IFNα after being administered the siRNA.

In some embodiments of the aforementioned methods, the methods further comprise administering to the subject a NRTI. In some embodiments of the aforementioned methods, the subject being administered the siRNA has been administered a NRTI prior to the administration of the siRNA. In some embodiments, the subject has been administered a NRTI for at least 2 months, at least 3 months, at least 4 months, at least 5 months, or at least 6 months prior to the administration of the siRNA. In some embodiments, the subject has been administered a NRTI for at least 2 months prior to the administration of the siRNA. In some embodiments, the subject has been administered a NRTI for at least 6 months prior to the administration of the siRNA. In some embodiments, the subject is administered a NRTI during the same period of time that the subject is administered the siRNA. In some embodiments of the methods, the subject is subsequently administered NRTI after being administered the siRNA.

Some embodiments of the present disclosure provide an siRNA for use in the treatment of a chronic HBV infection in a subject, wherein the siRNA has a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Agn) is adenosine-glycol nucleic acid (GNA); s is a phosphorothioate linkage; and L96 is N-[tris(GalNAc-alkyl)-amidodecanoyl)]-4-hydroxyprolinol. In some embodiments of the siRNA for use, the subject is also administered a PEG-IFNα. In some embodiments, the siRNA and PEG-IFNα are administered to the subject over the same time period. In some embodiments, the siRNA is administered to the subject for a period of time before the PEG-IFNα is administered to the subject. In some embodiments, the PEG-IFNα is administered to the subject for a period of time before the siRNA is administered to the subject. In some embodiments, the subject has been administered PEG-IFNα prior to the administration of the siRNA. In some embodiments, the subject is administered PEG-IFNα during the same period of time that the subject is administered the siRNA. In some embodiments, the subject is subsequently administered PEG-IFNα. In any of the aforementioned siRNAs for use, the subject may also be administered a NRTI or have previously been administered a NRTI. In some embodiments, the subject has been administered a NRTI prior to the administration of the siRNA. In some embodiments, the subject has been administered a NRTI for at least 2 months, at least 3 months, at least 4 months, at least 5 months, or at least 6 months prior to the administration of the siRNA. In some embodiments, the subject has been administered a NRTI for at least 2 months prior to the administration of the siRNA. In some embodiments, the subject has been administered a NRTI for at least 6 months prior to the administration of the siRNA. In some embodiments, the subject is administered a NRTI during the same period of time that the subject is administered the siRNA. In some embodiments, the subject is subsequently administered a NRTI.

Some embodiments of the present disclosure provides the use of an siRNA in the manufacture of a medicament for the treatment of a chronic HBV infection, wherein the siRNA has a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Agn) is adenosine-glycol nucleic acid (GNA); s is a phosphorothioate linkage; and L96 is N-[tris(GalNAc-alkyl)-amidodecanoyl)]-4-hydroxyprolinol.

Some embodiments of the present disclosure provides the use of an siRNA and PEG-IFNα in the manufacture of a medicament for the treatment of a chronic HBV infection, wherein the siRNA has a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Agn) is adenosine-glycol nucleic acid (GNA); s is a phosphorothioate linkage; and L96 is N-[tris(GalNAc-alkyl)-amidodecanoyl)]-4-hydroxyprolinol.

Some embodiments of the present disclosure provides the use of an siRNA, PEG-IFNα, and an NRTI in the manufacture of a medicament for the treatment of a chronic HBV infection, wherein the siRNA has a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Agn) is adenosine-glycol nucleic acid (GNA); s is a phosphorothioate linkage; and L96 is N-[tri s(GalNAc-alkyl)-amidodecanoyl)]-4-hydroxyprolinol.

In some embodiments of the aforementioned methods, compositions for use, or uses, the dose of the siRNA is 0.8 mg/kg, 1.7 mg/kg, 3.3 mg/kg, 6.7 mg/kg, or 15 mg/kg. In some embodiments of the aforementioned methods, compositions for use, or uses, the dose of the siRNA is 20 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg. In some embodiments of the aforementioned methods, compositions for use, or uses, the dose of the siRNA is 50 mg, 100 mg, 150 mg, 200 mg, 250 mg, 300 mg, 400 mg, or 450 mg. In some embodiments of the aforementioned methods, compositions for use, or uses, the dose of the siRNA is 200 mg. In some embodiments of the aforementioned methods, compositions for use, or uses, the dose of the siRNA is at least 200 mg.

In some embodiments of the aforementioned methods, compositions for use, or uses, the siRNA is administered weekly.

In some embodiments of the aforementioned methods, compositions for use, or uses, more than one dose of the siRNA is administered. For example, in some embodiments, two doses of the siRNA are administered, wherein the second dose is administered 2, 3, or 4 weeks after the first dose. In some particular embodiments, two doses of the siRNA are administered, wherein the second dose is administered 4 weeks after the first dose.

In some embodiments of the aforementioned methods, two, three, four, five, six, or more doses of the siRNA are administered. For example, in some embodiments, two 400-mg doses of the siRNA are administered to the subject. In some embodiments, six 200-mg doses of the siRNA are administered to the subject.

In some embodiments of the methods, compositions for use, or uses described herein, the method comprises:

(a) administering to the subject two or more doses of at least 200 mg of an siRNA having a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; (Agn) is adenosine-glycol nucleic acid (GNA); s is a phosphorothioate linkage; and L96 is N-[tris(GalNAc-alkyl)-amidodecanoyl)]-4-hydroxyprolinol; and

(b) administering to the subject a nucleoside/nucleotide reverse transcriptase inhibitor (NRTI);

wherein the subject is HBeAg negative or HBeAg positive.

In some embodiments, the method further comprises administering to the subject a PEG-IFNα.

In some embodiments of the aforementioned methods, compositions for use, or uses, the siRNA is administered via subcutaneous injection. In some embodiments, the siRNA comprises administering 1, 2, or 3 subcutaneous injections per dose.

In some embodiments of the aforementioned methods, compositions for use, or uses, the dose of the PEG-IFNα is 50 μg, 100 μg, 150 μg, or 200 μg. In some embodiments, the dose of the PEG-IFNα is 180 μg.

In some embodiments of the aforementioned methods, compositions for use, or uses, the PEG-IFNα is administered weekly.

In some embodiments of the aforementioned methods, compositions for use, or uses, the PEG-IFNα is administered via subcutaneous injection.

In some embodiments of the aforementioned methods, compositions for use, or uses, the NRTI may be tenofovir, tenofovir disoproxil fumarate (TDF), tenofovir alafenamide (TAF), lamivudine, adefovir, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, ganciclovir, besifovir (ANA-380/LB-80380), or tenofvir-exaliades (TLX/CMX157). In some embodiments, the NRTI is entecavir (ETV). In some embodiments, the NRTI is tenofovir. In some embodiments, the NRTI is lamivudine. In some embodiments, the NRTI is adefovir or adefovir dipivoxil.

In some embodiments of the aforementioned methods, compositions for use, or uses, the subject is HBeAg negative. In some embodiments, the subject is HBeAg positive.

The siRNA can be present either in the same pharmaceutical composition as the other active agents, or the active agents may be present in different pharmaceutical compositions. Such different pharmaceutical compositions may be administered either combined/simultaneously or at separate times or at separate locations (e.g., separate parts of the body).

### IV. Kits for HBV Therapy

Also provided herein are kits including components of the HBV therapy. The kits may include an siRNA (e.g., HBV02) and, optionally one or both of (a) PEGIFNα and (b) a NRTI (e.g., entecavir, tenofovir, lamivudine, or adefovir or adefovir dipivoxil). Kits may additionally include instructions for preparing and/or administering the components of the HBV combination therapy.

Some embodiments of the present disclosure provide a kit comprising: a pharmaceutical composition comprising an siRNA according to any of the preceding claims, and a pharmaceutically acceptable excipient; and a pharmaceutical composition comprising PEG-IFNα, and a pharmaceutically acceptable excipient. In some embodiments, the kit further comprises a NRTI, and a pharmaceutically acceptable excipient.

### EXAMPLES

### Example 1

### Treatment of Chronic HBV Infection with HBV02

Safety, tolerability, pharmacokinetics (PK), and antiviral activity of HBV02 are evaluated in a Phase 1/2, randomized, double-blind, placebo-controlled clinical study. The study includes three parts. Part A is a single ascending dose design in healthy volunteers. Parts B and C are multiple ascending dose designs in subjects with chronic HBV on nucleos(t)ide reverse transcriptase inhibitor (NRTI) therapy. Subjects in Part B are HBeAg negative; subjects in Part C are HBeAg positive. HBeAg positivist reflects high levels of active replication of the virus in a person's liver cells.

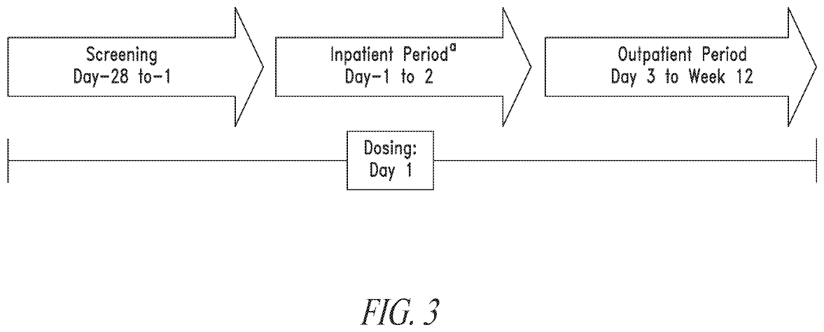

In Part A, a single dose of HBV02 is administered to healthy adult subjects. Each dose can consist of up to 2 subcutaneous (SC) injections based on assigned dose-level. Four dose-level cohorts are included in Part A: 50 mg, 100 mg, 200 mg, and 400 mg. Two sentinel subjects are randomized 1:1 to HBV02 or placebo. The sentinel subjects are dosed concurrently and monitored for 24 hours; if the investigator has no safety concerns, the remainder of the subjects in the same cohort are dosed. The remaining subjects will be randomized 5:1 to HBV02 or placebo. Two optional cohorts in Part A may be added following the same stratification, including sentinel dosing, up to a maximum dose of 900 mg. In addition to the optional cohorts, a total of 8 “floater” subjects may be added to expand any cohort in Part A. “Floater” subjects are to be added in increments of 4 and randomized 3:1 to HBV02 or placebo. The Part A dose escalation plan is shown in Table 3. The single ascending dose design for Part A is shown in FIG. 3.

TABLE 3
Part A Dose Escalation Plan.

| | Weight-based | Fixed dose^{a} | Dose Escalation |
|---|---|---|---|
| Cohort | dose (mg/kg) | (mg) | Factor |
| 1a | 0.8 | 50 | — |
| 2a | 1.7 | 100 | 2.0-fold |
| 3a | 3.3 | 200 | 2.0-fold |
| 4a | 6.7 | 400 | 2.0-fold |
| Optional: 5a and 6a | Up to 15 | Up to 900 | Up to 2.25-fold |

^{a}Based on average adult weight of 60 kg

Data from Part A are reviewed prior to initiating the dose-level cohort in subjects with chronic HBV infection. The cohort dosing strategy for Part B/C of this study is staggered; 2 dose levels in Part A (la: 50 mg and 2a: 100 mg) are completed and data reviewed before beginning dosing at the starting dose in Part B (lb: 50 mg). Part C is initiated at the Part C starting dose (3c: 200 mg) at the same time that the equivalent Part B dose level cohort is initiated (3b: 200 mg).

Subjects in Part B are non-cirrhotic adult subjects with HBeAg-negative chronic HBV infection, and have been on NRTI therapy for ≥6 months and have serum HBV DNA levels <90 IU/mL. To exclude the presence of fibrosis or cirrhosis, screening includes a noninvasive assessment of liver fibrosis, such as a FibroScan evaluation, unless the subject has results from a FibroScan evaluation performed within 6 months prior to screening or a liver biopsy performed within 1 year prior to screening that confirms the absence of Metavir F3 fibrosis or F4 cirrhosis.

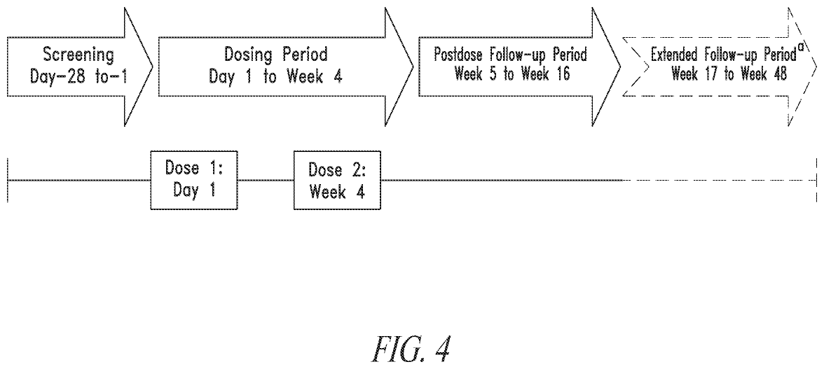

Two doses of HBV02 are administered to subjects 4 weeks apart. Each dose can consist of up to 2 SC injections based on assigned dose-level. Three dose-level cohorts are included in Part B, 50 mg, 100 mg, and 200 mg, such that the cumulative dose received for subjects in Part B is 100 mg, 200 mg, and 400 mg. Each cohort is randomized 3:1 to HBV02 or placebo. Two optional cohorts in Part B may be added following the same stratification, by a factor of 1.5-fold, up to a maximum of 450 mg per dose (900 mg cumulative dose). In addition to the optional cohorts, a total of 16 “floater” subjects may be added to expand any cohort in Part B. “Floater” subjects are to be added in increments of 4 and randomized 3:1 to HBV02 or placebo. Cohort 1b is initiated after cumulative review of all available safety data, inclusive of the Week 4 laboratory and clinical data of the last available healthy volunteer subject in the 100 mg cohort (Cohort 2a). The dose escalation plan for Parts B and C is shown in Table 4. The multiple ascending dose design for Part B/C is shown in FIG. 4.

Subjects in Part C are non-cirrhotic adult subjects with HBeAg-positive chronic HBV infection, and have been on NRTI therapy for ≥6 months and have serum HBV DNA levels <90 IU/mL. To exclude the presence of fibrosis or cirrhosis, screening includes a noninvasive assessment of liver fibrosis, such as a FibroScan evaluation, unless the subject has results from a FibroScan evaluation performed within 6 months prior to screening or a liver biopsy performed within 1 year prior to screening that confirms the absence of Metavir F3 fibrosis or F4 cirrhosis Two doses of HBV02 are administered to subjects 4 weeks apart. Each dose can consist of up to 2 SC injections based on assigned dose-level. To accommodate the anticipated lower prevalence of HBeAg-positive patients on NRTI therapy, only 1 dose level cohort (200 mg) is planned for HBeAg-positive subjects. Part C includes one dose-level cohort, 200 mg, such that the cumulative dose received for subjects in Part C is 400 mg. The cohort is randomized 3:1 to HBV02 or placebo. Two optional cohorts in Part C may be added following the same stratification, by a factor of 1.5-fold, up to a maximum of 450 mg per dose (900 mg cumulative dose). In addition to the optional cohorts, a total of 16 “floater” subjects may be added to expand any cohort in Part C. “Floater” subjects are to be added in increments of 4 and randomized 3:1 to HBV02 or placebo. The only planned cohort in Part C, Cohort 3c, is initiated at the same time as Cohort 3b after review of all available safety data inclusive of Week 6 clinical and laboratory data from Cohort 2b. Subjects in Cohort 3c receive HBV02 at the same dose level as subjects in Cohort 3b (200 mg administered twice at a dosing interval of 4 weeks).

TABLE 4
Part B/C Dose Escalation Plan.

| | Weight-based | Fixed dose^{a} | Dose Escalation |
|---|---|---|---|
| Cohort | dose (mg/kg) | (mg) | Factor |
| 1b | 0.8 | 50 | — |
| 2b | 1.7 | 100 | 2.0-fold |
| 3b and 3c | 3.3 | 200 | 2.0-fold |
| Optional: 4b and 4c | Up to 5 | Up to 300 | Up to 1.5-fold |
| Optional: 5b and 6c | Up to 7.5 | Up to 450 | Up to 1.5-fold |

^{a}Based on average adult weight of 60 kg

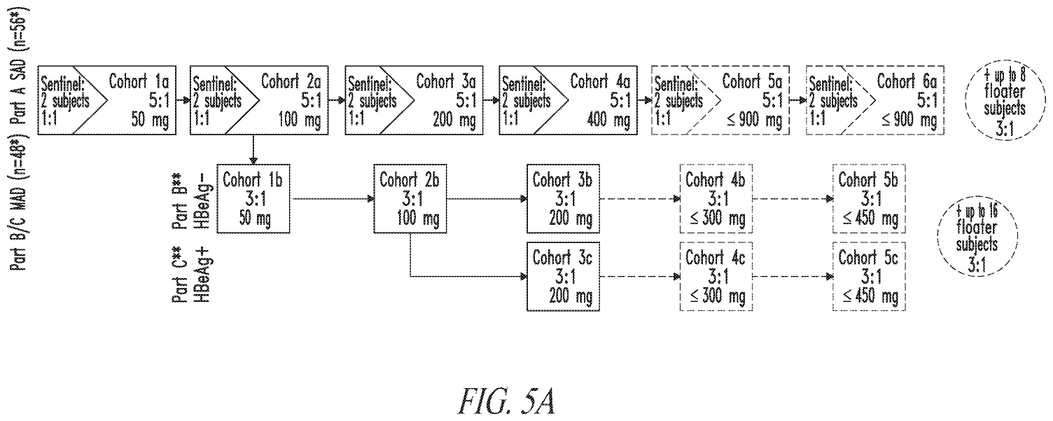

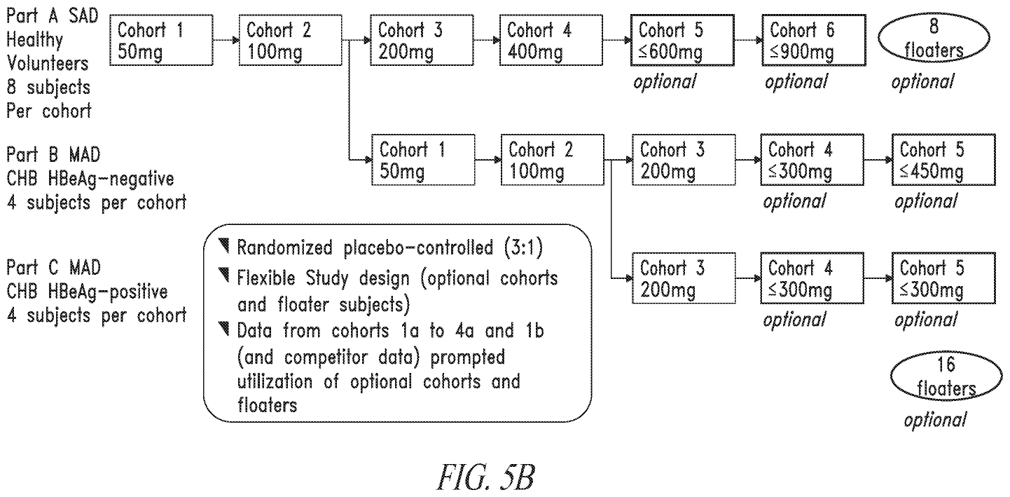

Summaries of the study drug dosing and administration for Parts A-C are shown in Table 5 and FIGS. 5A and 5B.

TABLE 5
Study Drug Dose and Administration

| | Visit | Visit | | | | Cumulative |
|---|---|---|---|---|---|---|
| | Dose | Dose | Cumulative | Injections | | Dose |
| | Level | Volume | Dose | Per Dose | Injections | Volume |
| Cohort | (mg) | (mL) | (mg) | Administration | Total | (mL)^{a} |
| 1a | 50 | 0.25 | 50 | 1 | 1 | 0.25 |
| 2a | 100 | 0.50 | 100 | 1 | 1 | 0.50 |
| 3a | 200 | 1.0 | 200 | 1 | 1 | 1.0 |
| 4a | 400 | 2.0 | 400 | 2 | 2 | 2.0 |
| Optional: | ≤900 | ≤4.5 | ≤900 | 3 | 3 | ≤4.5 |
| 5a | | | | | | |
| Optional: | ≤900 | ≤4.5 | ≤900 | 3 | 3 | ≤4.5 |
| 6a | | | | | | |
| 1b | 50 | 0.25 | 100 | 1 | 2 | 0.50 |
| 2b | 100 | 0.50 | 200 | 1 | 2 | 1.0 |
| 3b | 200 | 1.0 | 400 | 1 | 2 | 2.0 |
| Optional: | ≤300 | ≤1.5 | ≤600 | 1 | 2 | ≤3 |
| 4b | | | | | | |
| Optional: | ≤450 | ≤2.5 | ≤900 | 2 | 4 | ≤5 |
| 5b | | | | | | |
| 3c | 200 | 1.0 | 400 | 1 | 2 | 2.0 |
| Optional: | <300 | ≤1.5 | ≤600 | 1 | 2 | ≤3 |
| 4c | | | | | | |
| Optional: | <450 | ≤2.5 | ≤900 | 2 | 4 | ≤5 |
| 5c | | | | | | |

^{a}Injection volume per site not exceeding 1.5 mL

HBV02 is supplied as a sterile solution for SC injection at a free acid concentration of 200 mg/mL. The placebo is sterile, preservative-free normal saline 0.9% solution for SC injection.

Following administration of HBV02 or placebo and any adverse effects are noted. PK parameters of HBV02 and possible metabolites are also measured and may include plasma: maximum concentration, time to reach maximum concentration, area under the concentration versus time curve [to last measurable timepoint and to infinity], percent of area extrapolated, apparent terminal elimination half-life, clearance, and volume of distribution; urine: fraction eliminated in the urine and renal clearance. The following are also determined: maximum reduction of serum HBsAg from Day 1 until Week 16; number of subjects with serum HBsAg loss at any timepoint; number of subjects with sustained serum HBsAg loss for ≥6 months; number of subjects with anti-HBs seroconversion at any timepoint; number of subjects with HBeAg loss and/or anti-HBe seroconversion at any timepoint (for HBeAg-positive subjects in Part C only); assessment of the effect of HBV02 on other markers of HBV infection including detection of serum HBcrAg, HBV RNA, and HBV DNA; and evaluation of potential biomarkers for host responses to infection and/or therapy, including genetic, metabolic, and proteomic parameters. In order to evaluate the PK parameters, blood samples are collected predose (≤15 min prior to dosing), and then 30 min, 1 hr, 2 hr, 4 hr, 6 hr, 8 hr, 10 hr, 12 hr, 24 hr, and 48 hr after dosing; and urine samples are collected predose (≤15 min prior to dosing), and then collected and pooled for 0-4 hr, 4-8 hr, 8-12 hr, 12-24 hr, 48 hr, and 1 week after dosing. For subjects in Parts B or C, blood samples for measuring HBsAg, anti-HBs, HBeAg, anti-HBe, HBV DNA, HBV RNA, or HBcrAg may be collected at one or more of the following timepoints: screening (28 days to 1 day before dosing), day 1 (dosing), day 2 (after dosing), weekly during the dosing period, weekly for 4 weeks post-dosing, 12 weeks after dosing, 16 weeks after dosing, 20 weeks after dosing, and 24 weeks after dosing.

Fasting is not required for the study procedures.

### Example 2

### Treatment of Chronic HBV with HBV02 Alone or in Combination with PEG-IFNα

Safety, tolerability, pharmacokinetics, and antiviral activity of HBV02 alone or in combination with PEG-IFNα are evaluated in a Phase ½ clinical study. The study includes four parts. Parts A-C are a randomized, double-blind, placebo-controlled clinical study of HBV02 administered subcutaneously to healthy adult subjects or non-cirrhotic adult subjects with chronic HBV infection who are on NRTI therapy. Part A is a single ascending dose design in healthy volunteers. Parts B and C are multiple ascending dose designs in non-cirrhotic subjects with chronic HBV on NRTI therapy. Subjects in Part B are HBeAg negative; subjects in Part C are HBeAg positive. HBeAg positivity reflects high levels of active replication of the virus in a person's liver cells. Part D is a randomized, open-label Phase 2 study of HBV02 administered alone or in combination with PEG-IFNα in non-cirrhotic adult subjects with chronic HBV on NRTI therapy; Part D includes HBeAg-positive and HBeAg-negative subjects.

In Part A, a single dose of HBV02 is administered to healthy adult subjects. Each dose can consist of up to 3 subcutaneous (SC) injections based on assigned dose-level. Four dose-level cohorts are included in Part A: 50 mg, 100 mg, 200 mg, and 400 mg. Two sentinel subjects are randomized 1:1 to HBV02 or placebo. The sentinel subjects are dosed concurrently and monitored for 24 hours; if the investigator has no safety concerns, the remainder of the subjects in the same cohort are dosed. The remaining subjects will be randomized 5:1 to HBV02 or placebo. Two optional cohorts in Part A may be added following the same stratification, including sentinel dosing, up to a maximum dose of 900 mg. In addition to the optional cohorts, a total of 8 “floater” subjects may be added to expand any cohort in Part A. “Floater” subjects are to be added in increments of 4 and randomized 3:1 to HBV02 or placebo. The single ascending dose design for Part A is shown in FIG. 3.

Subjects in Part B are non-cirrhotic adult subjects with HBeAg-negative chronic HBV infection, and have been on NRTI therapy for ≥6 months and have serum HBV DNA levels <90 IU/mL. To exclude the presence of fibrosis or cirrhosis, screening includes a noninvasive assessment of liver fibrosis, such as a FibroScan evaluation. Two doses of HBV02 are administered to subjects 4 weeks apart. Each dose can consist of up to 2 SC injections based on assigned dose-level. Three dose-level cohorts are included in Part B, 50 mg, 100 mg, and 200 mg, such that the cumulative dose received for subjects in Part B is 100 mg, 200 mg, and 400 mg. Each cohort is randomized 3:1 to HBV02 or placebo. To accommodate the anticipated lower prevalence of HBeAg-positive patients on NRTI therapy, only 1 dose level cohort (200 mg) is planned for HBeAg-positive subjects. Two optional cohorts in Part B may be added following the same stratification, up to a maximum of 450 mg per dose (900 mg cumulative dose). In addition to the optional cohorts, a total of 16 “floater” subjects may be added to expand any cohort in Part B. “Floater” subjects are to be added in increments of 4 and randomized 3:1 to HBV02 or placebo. Cohort 1b is initiated after cumulative review of all available safety data, inclusive of the Week 4 laboratory and clinical data of the last available healthy volunteer subject in the 100 mg cohort (Cohort 2a). The dose escalation plan for Parts B and C is shown in Table 5. The multiple ascending dose design for Part B/C is shown in FIG. 4.

Subjects in Part C are non-cirrhotic adult subjects with HBeAg-positive chronic HBV infection, and have been on NRTI therapy for ≥6 months and have serum HBV DNA levels <90 IU/mL. Two doses of HBV02 are administered to subjects 4 weeks apart. Each dose can consist of up to 2 SC injections based on assigned dose-level. Part C includes one dose-level cohort, 200 mg, such that the cumulative dose received for subjects in Part C is 400 mg. The cohort is randomized 3:1 to HBV02 or placebo. Two optional cohorts in Part C may be added following the same stratification, up to a maximum of 450 mg per dose (900 mg cumulative dose). In addition to the optional cohorts, a total of 16 “floater” subjects may be added to expand any cohort in Part C. “Floater” subjects are to be added in increments of 4 and randomized 3:1 to HBV02 or placebo.

Summaries of the study drug dosing and administration for Parts A-C are shown in Table 5 and FIGS. 5A and 5B.

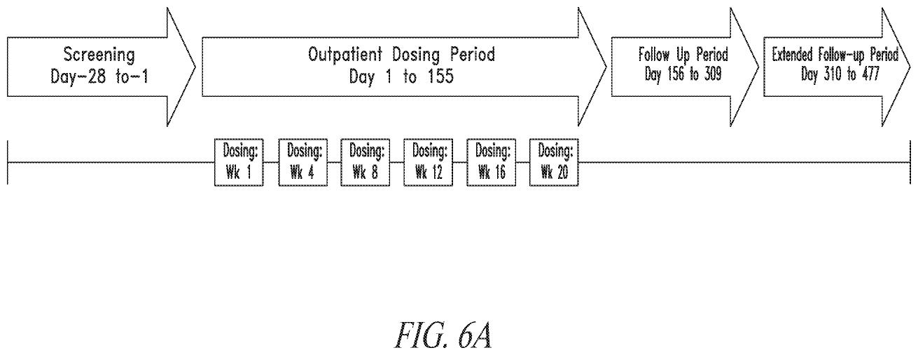

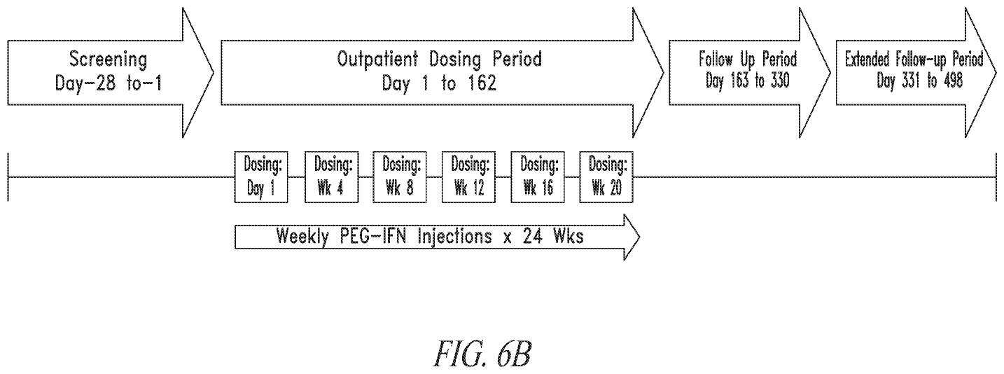

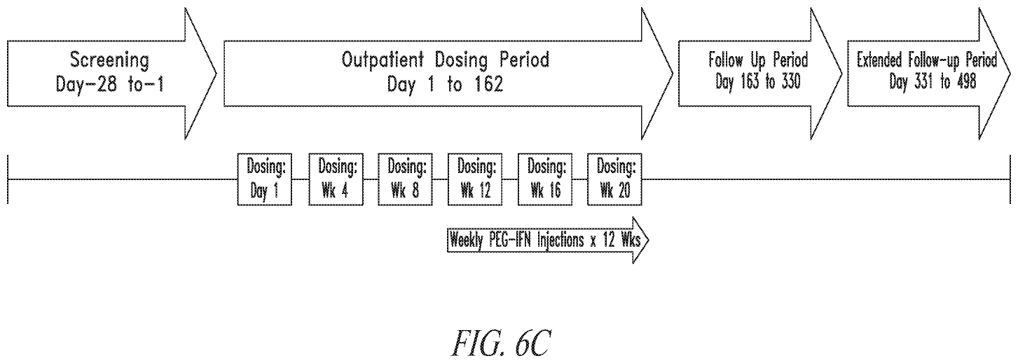

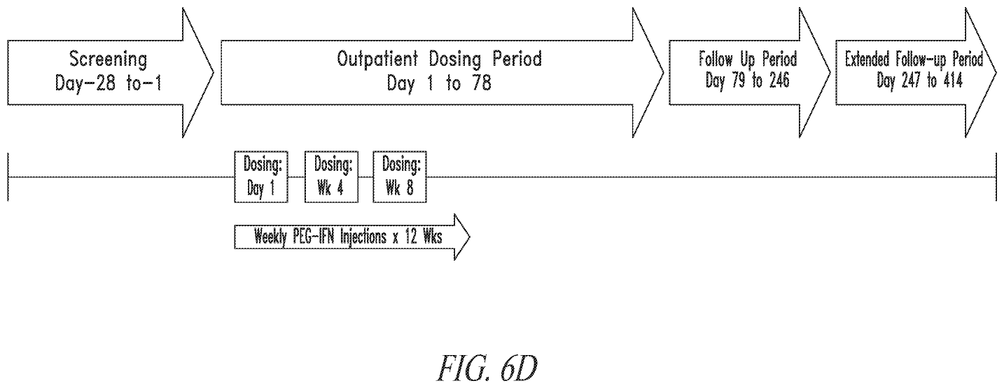

Subjects in Part D are non-cirrhotic adult subjects with HBeAg-positive or HBeAg-negative chronic HBV infection, and have been on NRTI therapy for ≥2 months and have serum HBV DNA levels <90 IU/mL and serum HBsAg levels >50 IU/mL. Dose level and number of doses of HBV02 in Part D is determined based on the safety and tolerability of HBV02 in Parts A-C and analysis of antiviral activity of HBV02 in Parts B and C. The dose level in Part D does not exceed the highest dose level evaluated in Parts B and C, and the number of doses will be up to 6 doses (e.g., between 3 and 6 doses) administered every 4 weeks. Subjects are randomized to one of Cohort 1d, Cohort 2d, Cohort 3d, and Cohort 4d (optional) (e.g., 100 subjects total, 25 subjects per cohort). In Cohort 1d, up to 6 doses (e.g. , 3 to 6 doses) of HBV02 are administered to subjects at a frequency of every 4 weeks. Each subject receives a dose of HBV02 on day 1, week 4, and week 8 and may receive additional doses at weeks 12, 16, and 20. In Cohort 2d, up to 6 (e.g. , 3 to 6 doses) of HBV02 are administered to subjects 4 weeks apart, and PEG-IFNα is administered for 24 weekly doses (i.e., each dose given 1 week apart), starting on day 1. Each subject receives a dose of HBV02 on day 1, week 4, and week 8 and may receive additional doses at weeks 12, 16, and 20. In Cohort 3d, up to 6 (e.g. , 3 to 6 doses) of HBV02 are administered to subjects 4 weeks apart, and PEG-IFNα is administered for 12 weekly doses (i.e., each dose given 1 week apart), starting at week 12. Each subject receives a dose of HBV02 on day 1, week 4, and week 8 and may receive additional doses at weeks 12, 16, and 20. In Cohort 4d, 3 doses of HBV02 are administered to subjects 4 weeks apart, and PEG-IFNα is administered for 12 weekly doses (i.e., each dose given 1 week apart), starting at day 1. Each subject receives a dose of HBV02 on day 1, week 4, and week 8. The doses of PEG-INFα administered to subjects in Cohorts 2d, 3d, and 4d is 180 administered by SC injection. FIGS. 6A-6D are schematics illustrating the study designs for Part D. The drug administration schedule for cohort 4d is shown in Table 6.

TABLE 6
Cohort 4d Study Drug Administration Schedule (D 1 = Day 1, W 1 = Week 1, etc.).

| | D 1 | W 1 | W 2 | W 3 | W 4 | W 5 | W 6 | W 7 | W 8 | W 9 | W 10 | W 11 |
|---|---|---|---|---|---|---|---|---|---|---|---|---|
| HBV02 | X | | | | X | | | | X | | | |
| PEG- | X | X | X | X | X | X | X | X | X | X | X | X |
| INFα^{a} | | | | | | | | | | | | |

^{a}Subjects who discontinue from PEG-IFNα treatment due to PEG-IFNα-related adverse reactions may continue to receive treatment with HBV02.

To exclude the presence of cirrhosis, screening of subjects enrolled in Part B/C and Part D includes a noninvasive assessment of liver fibrosis such as a FibroScan evaluation, unless the subject has results from a FibroScan evaluation performed within 6 months prior to screening or a liver biopsy performed within 1 year prior to screening that confirms the absence of Metavir F3 fibrosis or F4 cirrhosis.

HBV02 is supplied as a sterile solution for SC injection at a free acid concentration of 200 mg/mL. The placebo is sterile, preservative-free normal saline 0.9% solution for SC injection.

Following administration of HBV02 or placebo and any adverse effects are noted. PK parameters of HBV02 and possible metabolites are also measured and may include plasma: maximum concentration, time to reach maximum concentration, area under the concentration versus time curve [to last measurable timepoint and to infinity], percent of area extrapolated, apparent terminal elimination half-life, clearance, and volume of distribution; urine: fraction eliminated in the urine and renal clearance. The following are also determined: maximum reduction of serum HBsAg from Day 1 until Week 16; number of subjects with serum HBsAg loss at any timepoint; number of subjects with sustained serum HBsAg loss for ≥6 months; number of subjects with anti-HBs seroconversion at any timepoint; number of subjects with HBeAg loss and/or anti-HBe seroconversion at any timepoint (for HBeAg-positive subjects in Part C and Part D only); assessment of the effect of HBV02 on other markers of HBV infection including detection of serum HBcrAg, HBV RNA, and HBV DNA; and evaluation of potential biomarkers for host responses to infection and/or therapy, including genetic, metabolic, and proteomic parameters.

Data from Part A are reviewed prior to initiating the dose-level cohort in subjects with chronic HBV infection. The cohort dosing strategy for Part B/C of this study is staggered; 2 dose levels in Part A (la: 50 mg and 2a: 100 mg) are completed and data reviewed before beginning dosing at the starting dose in Part B (lb: 50 mg). Part C is initiated at the Part C starting dose (3c: 200 mg) at the same time that the equivalent Part B dose level cohort is initiated (3b: 200 mg).

Fasting is not required for the study procedures.

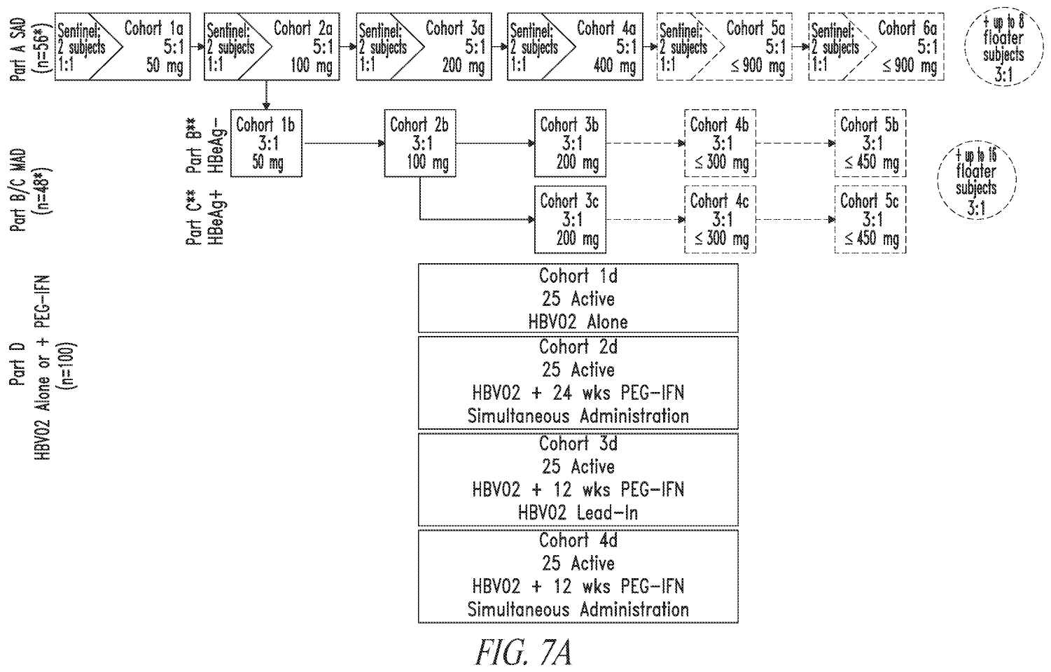

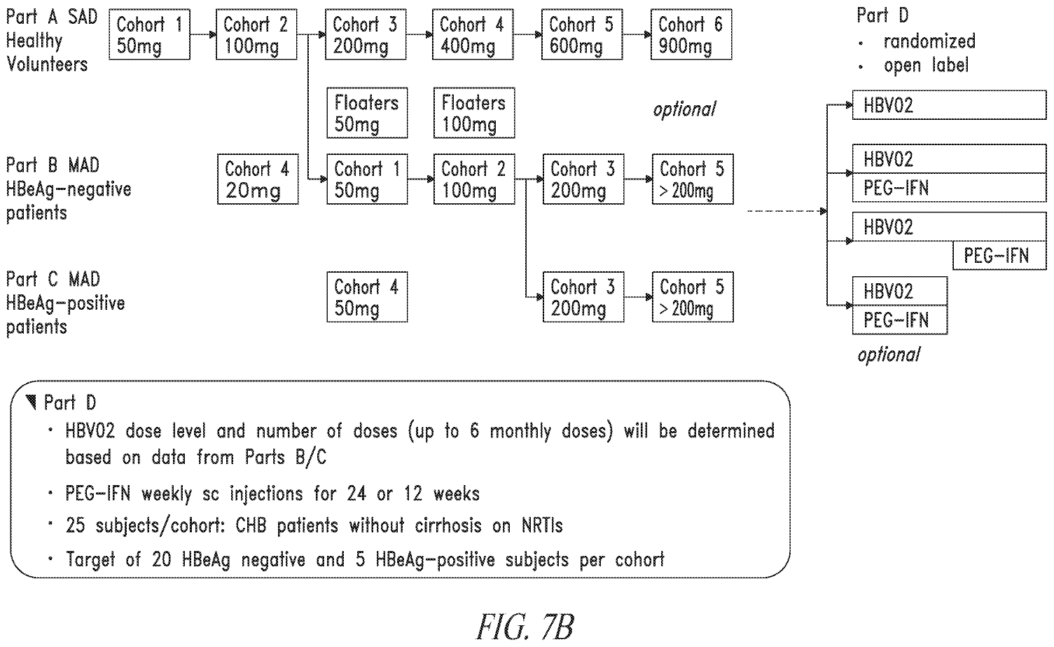

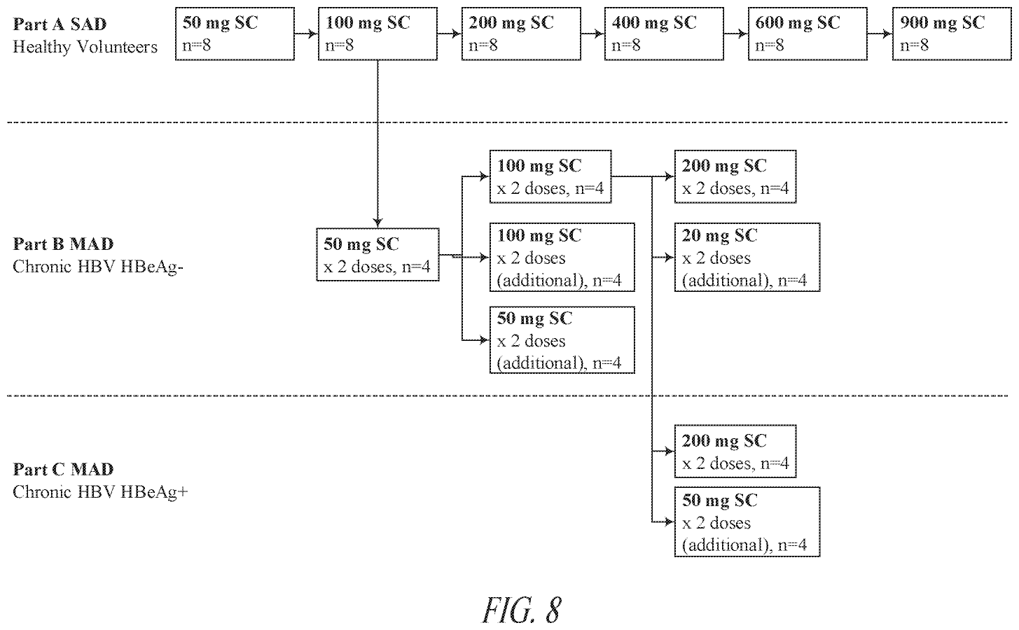

FIGS. 7A and 7B show the study design for Parts A-D.

### Example 3

### Treatment of Chronic HBV with HBV02 Alone or in Combination with PEG-IFNα

Safety, tolerability, pharmacokinetics, and antiviral activity of HBV02 were evaluated in a Phase ½ clinical study. The study includes four parts. Parts A-C are a randomized, double-blind, placebo-controlled clinical study of HBV02 administered subcutaneously to healthy adult subjects or non-cirrhotic adult subjects with chronic HBV infection who are on NRTI therapy. Part A is a single ascending dose design in healthy volunteers. Parts B and C are multiple ascending dose designs in non-cirrhotic subjects with chronic HBV on NRTI therapy. Subjects in Part B are HBeAg negative; subjects in Part C are HBeAg positive. HBeAg positivity reflects high levels of active replication of the virus in a person's liver cells. HBeAg positive patients are generally younger, and thought to have more preserved immune function, as compared to HBeAg negative patients who are generally older and have experienced greater immune exhaustion. HBeAg negative patients are also thought to have larger amounts of integrated DNA compared to HBeAg positive patients. Part D is a randomized, open-label Phase 2 study of HBV02 administered alone or in combination with PEG-IFNα in non-cirrhotic adult subjects with chronic HBV on NRTI therapy; Part D includes HBeAg-positive and HBeAg-negative subjects.

i. Preliminary Animal Dosing Study

Doses of HBV02 used in the study were determined by calculating the human equivalent doses (HEDs) of the no observed adverse effect levels (NOAELs) in animal toxicology studies and applying a safety margin to those HEDs Body surface area (m/kg²) conversion factors were used to calculate HEDs of animal doses. No toxicity was observed in a rat Good Laboratory Practice (GLP) study after 3 biweekly doses of HBV02 at the highest dose tested, 150 mg/kg, corresponding to a HED of 24 mg/kg/dose (Table 7). No toxicity was observed in a non-human primate (NHP) GLP study after 3 biweekly doses of HBV02 at the highest dose tested, 300 mg/kg, corresponding to a HED of 97 mg/kg/dose (Table 7). Using this methodology, the proposed starting dose of 0.8 mg/kg in humans represents the 30-fold safety margin of the HED of the NOAEL projected in rats, and the 120-fold safety margin of the HED of the NOAEL projected in NHPs. Other siRNAs using the GalNAc platform have demonstrated meaningful liver target engagement at 1 to 15 mg/kg. Furthermore, a statistically significant decline in HBsAg in preclinical HBV mouse models at a dose range of 1 to 9 mg/kg was observed.

TABLE 7
Proposed Starting Dose for HBV02.

| | NOAEL | HED | Starting Dose |
|---|---|---|---|
| Study Species and Duration | (mg/kg) | (mg/kg) | (mg/kg) |
| Cynomolgus monkey | 300 | 97 | 0.8 |
| 4-week study (3 biweekly doses) | | | (120-fold safety |
| followed by 13-week recovery | | | margin) |
| Rat | 150 | 24 | 0.8 |
| 4-week study (3 biweekly doses) | | | (30-fold safety |
| followed by 13-week recovery | | | margin) |

A fixed dose of HBVO2 was used in the clinical study because HBV02, like other GalNAc-conjugated siRNAs, is taken up by the liver and minimally distributed to other organs and tissues. Therefore, weight-based dosing is not anticipated to reduce the inter-individual variation in the pharmacokinetics (PK) of HBV02 in adults and a fixed dose has the advantage of avoiding potential dose calculation errors.

ii. Methods

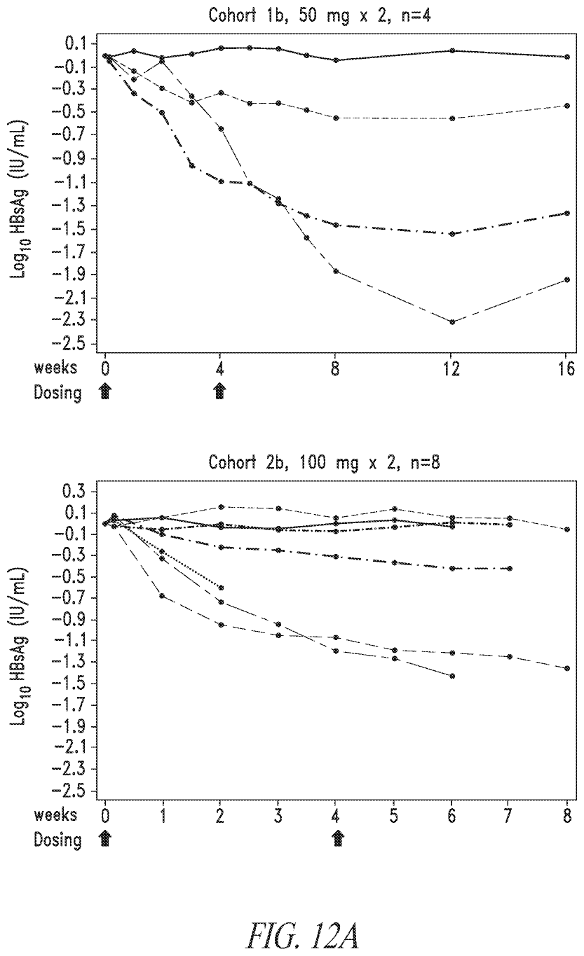

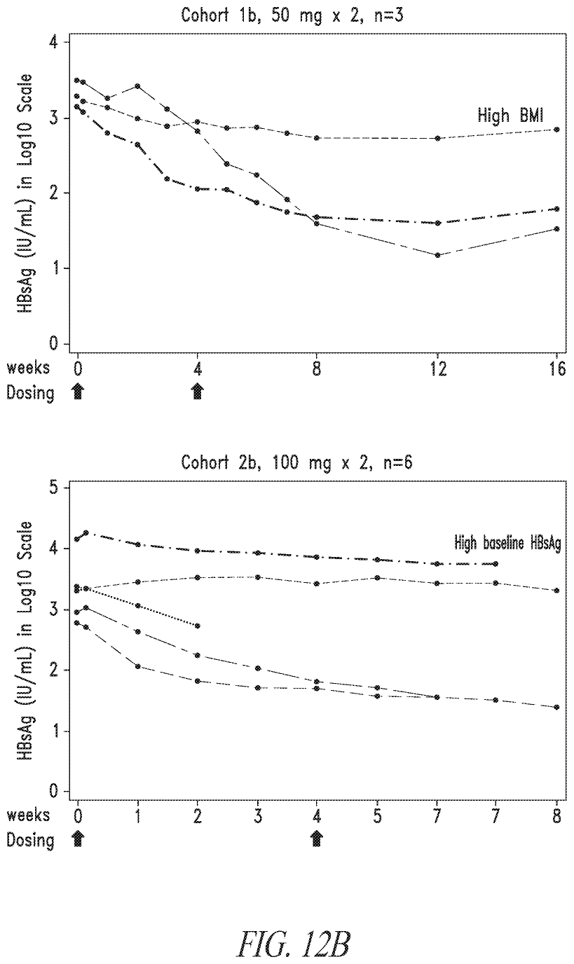

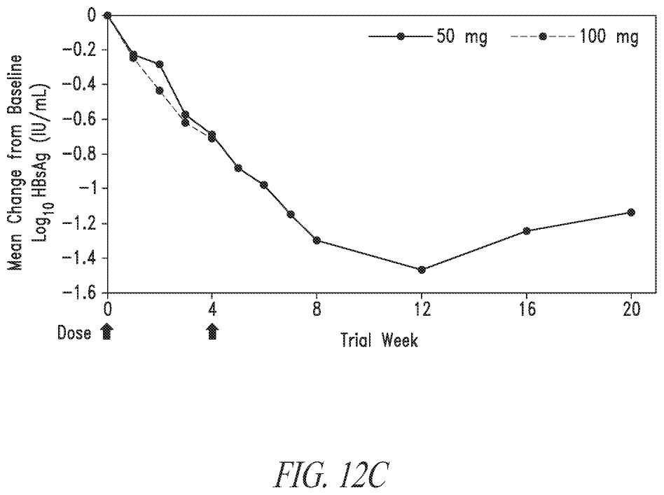

The study design is shown in FIG. 12.

In Part A, a single dose of HBV02 was administered to healthy adult subjects. Each dose consisted of up to 3 subcutaneous (SC) injections based on assigned dose-level. Six dose-level cohorts were included in Part A: 50 mg, 100 mg, 200 mg, 400 mg, 600 mg, and 900 mg. Two sentinel subjects were randomized 1:1 to HBV02 or placebo. The sentinel subjects were dosed concurrently and monitored for 24 hours; if the investigator had no safety concerns, the remainder of the subjects in the same cohort were dosed.

Subjects in Part B were non-cirrhotic adult subjects with HBeAg-negative chronic HBV infection, and have been on NRTI therapy for ≥6 months and have serum HBV DNA levels <90 IU/mL. To exclude the presence of fibrosis or cirrhosis, screening included a noninvasive assessment of liver fibrosis. Two doses of HBV02 were administered to subjects 4 weeks apart (i.e., on Day 1 and Day 29). Each dose consisted of up to 2 SC injections based on assigned dose-level. Six cohorts were included in Part B, at doses of 20 mg, 50 mg, 100 mg, or 200 mg, such that the cumulative dose received for subjects in Part B was 40 mg, 100 mg, 200 mg, or 400 mg. Each cohort was randomized 3:1 to HBV02 or placebo. The 50 mg cohort of Part B was initiated after cumulative review of all available safety data, inclusive of the Week 4 laboratory and clinical data of the last available healthy volunteer subject in the 100 mg cohort.

Subjects in Part C were non-cirrhotic adult subjects with HBeAg-positive chronic HBV infection, and have been on NRTI therapy for ≥6 months and have serum HBV DNA levels <90 IU/mL. To accommodate the anticipated lower prevalence of HBeAg-positive patients on NRTI therapy, only 2 dose level cohorts (50 mg and 200 mg) were included for HBeAg-positive subjects. Two doses of HBV02 were administered to subjects 4 weeks apart (i.e., on Day 1 and Day 29). Each dose consisted of up to 2 SC injections based on assigned dose-level. Part C included two dose-level cohorts, 50 mg and 200 mg, such that the cumulative dose received for subjects in Part C was 100 mg or 400 mg. The cohort was randomized 3:1 to HBV02 or placebo.

Patients with chronic HBV who experienced a greater than 10% decline from baseline serum HBsAg at Week 16 in HBsAg were followed for up to 32 additional weeks.

Inclusion criteria for Parts B and C included: age 18-65 years; detectable serum HBsAg for ≥6 months; on NRTI therapy for ≥6 months; HBsAg >150 IU/mL; HBV DNA <90 IU/mL; and serum alanine aminotransferase (ALT) and aspartate aminotransferase (AST) ≤2×upper limit of normal (ULN). Exclusion criteria included: significant fibrosis or cirrhosis (FibroScan >8.5 kPa at screening or Metavir F3/F4 liver biopsy within 1 year); bilirubin, international normalized ratio (INR), or prothrombin time >ULN; active HIV, HCV, or hepatitis Delta virus infection; and creatinine clearance <60 mL/min (Cockcroft-Gault).

Subjects in Part D are non-cirrhotic adult subjects with HBeAg-positive or HBeAg-negative chronic HBV infection, and have been on NRTI therapy for ≥2 months and have serum HBV DNA levels <90 IU/mL and serum HBsAg levels >50 IU/mL. Dose level and number of doses of HBV02 in Part D is determined based on the safety and tolerability of HBV02 in Parts A-C and analysis of antiviral activity of HBV02 in Parts B and C. The dose level in Part D does not exceed the highest dose level evaluated in Parts B and C, and the number of doses will be up to 6 doses (e.g., between 3 and 6 doses) administered every 4 weeks. Subjects are randomized to one of Cohort 1d, Cohort 2d, Cohort 3d, and Cohort 4d (optional) (e.g., 100 subjects total, 25 subjects per cohort). In Cohort 1d, up to 6 doses (e.g. , 3 to 6 doses) of HBV02 are administered to subjects at a frequency of every 4 weeks. Each subject receives a dose of HBV02 on day 1, week 4, and week 8 and may receive additional doses at weeks 12, 16, and 20. In Cohort 2d, up to 6 (e.g. , 3 to 6 doses) of HBV02 are administered to subjects 4 weeks apart, and PEG-IFNα is administered for 24 weekly doses (i.e., each dose given 1 week apart), starting on day 1. Each subject receives a dose of HBV02 on day 1, week 4, and week 8 and may receive additional doses at weeks 12, 16, and 20. In Cohort 3d, up to 6 (e.g. , 3 to 6 doses) of HBV02 are administered to subjects 4 weeks apart, and PEG-IFNα is administered for 12 weekly doses (i.e., each dose given 1 week apart), starting at week 12. Each subject receives a dose of HBV02 on day 1, week 4, and week 8 and may receive additional doses at weeks 12, 16, and 20. In Cohort 4d, 3 doses of HBV02 are administered to subjects 4 weeks apart, and PEG-IFNα is administered for 12 weekly doses (i.e., each dose given 1 week apart), starting at day 1. Each subject receives a dose of HBV02 on day 1, week 4, and week 8. The doses of PEG-INFα administered to subjects in Cohorts 2d, 3d, and 4d is 180 administered by SC injection. FIGS. 6A-6D are schematics illustrating the study designs for Part D. The drug administration schedule for cohort 4d is shown in Table 8.

TABLE 8
Cohort 4d Study Drug Administration Schedule (D 1 = Day 1, W 1 = Week 1, etc.).

| | D 1 | W 1 | W 2 | W 3 | W 4 | W 5 | W 6 | W 7 | W 8 | W 9 | W 10 | W 11 |
|---|---|---|---|---|---|---|---|---|---|---|---|---|
| HBV02 | X | | | | X | | | | X | | | |
| PEG- | X | X | X | X | X | X | X | X | X | X | X | X |
| INFα^{a} | | | | | | | | | | | | |

^{a}Subjects who discontinue from PEG-IFNα treatment due to PEG-IFNα-related adverse reactions may continue to receive treatment with HBV02.

To exclude the presence of cirrhosis, screening of subjects enrolled in Parts B and C included a noninvasive assessment of liver fibrosis such as a FibroScan evaluation, unless the subject had results from a FibroScan evaluation performed within 6 months prior to screening or a liver biopsy performed within 1 year prior to screening that confirmed the absence of Metavir F3 fibrosis or F4 cirrhosis. The same methods are used to exclude cirrhotic subjects from inclusion in Part D.

HBV02 was supplied as a sterile solution for SC injection at a free acid concentration of 200 mg/mL. The placebo was sterile, preservative-free normal saline 0.9% solution for SC injection.

Following administration of HBV02 or placebo, any adverse effects were noted. PK parameters of HBV02 and possible metabolites were also measured and included plasma: maximum concentration, time to reach maximum concentration, area under the concentration versus time curve [to last measurable timepoint and to infinity], percent of area extrapolated, apparent terminal elimination half-life, clearance, and volume of distribution; urine: fraction eliminated in the urine and renal clearance. The following were also determined: maximum reduction of serum HBsAg from Day 1 until Week 16; number of subjects with serum HBsAg loss at any timepoint; number of subjects with sustained serum HBsAg loss for ≥6 months; number of subjects with anti-HBs seroconversion at any timepoint; number of subjects with HBeAg loss and/or anti-HBe seroconversion at any timepoint (for HBeAg-positive subjects in Part C and Part D only); assessment of the effect of HBV02 on other markers of HBV infection including detection of serum HBcrAg, HBV RNA, and HBV DNA; and evaluation of potential biomarkers for host responses to infection and/or therapy, including genetic, metabolic, and proteomic parameters. In order to evaluate the PK parameters for subjects in Part A, blood samples were collected predose (≤15 min prior to dosing), and then 30 min, 1 hr, 2 hr, 4 hr, 6 hr, 8 hr, 10 hr, 12 hr, 24 hr, and 48 hr after dosing; and urine samples were collected predose (≤15 min prior to dosing), and then collected and pooled for 0-4 hr, 4-8 hr, 8-12 hr, 12-24 hr, 48 hr, and 1 week after dosing. For subjects in Parts B or C, blood samples for measuring HBsAg, anti-HBs, HBeAg, anti-HBe, HBV DNA, HBV RNA, or HBcrAg were collected at one or more of the following timepoints: screening (28 days to 1 day before dosing), day 1 (dosing), day 2 (after dosing), weekly during the dosing period, weekly for 4 weeks post-dosing, 12 weeks after dosing, 16 weeks after dosing, 20 weeks after dosing, and 24 weeks after dosing.

Data from Part A were reviewed prior to initiating the dose-level cohort in subjects with chronic HBV infection. The cohort dosing strategy for Part B/C of this study was staggered; 2 dose levels in Part A (50 mg and 100 mg) were completed and data reviewed before beginning dosing at the starting dose in Part B (50 mg). Part C was initiated at the Part C starting dose (200 mg) at the same time that the equivalent Part B dose level cohort is initiated (200 mg).

Fasting was not required for the study procedures.

iii. Preliminary Results from Parts A and B

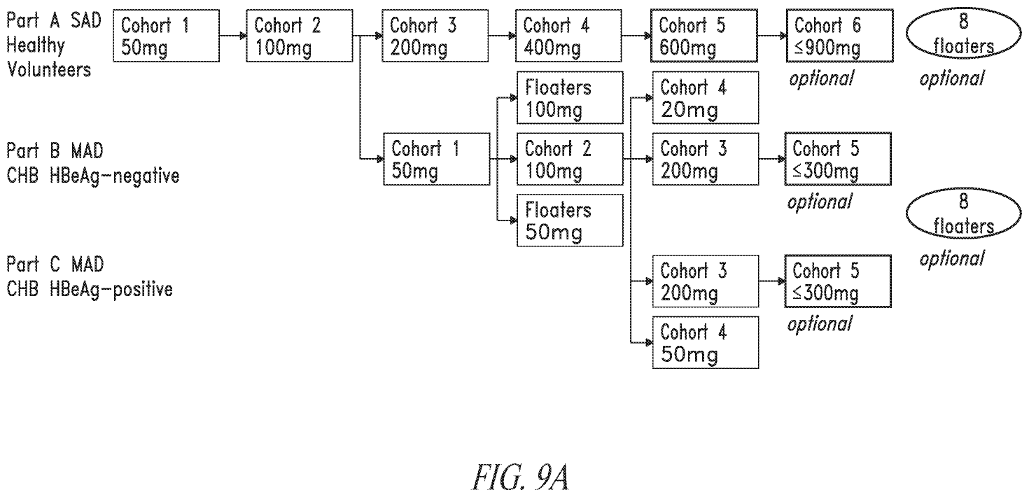

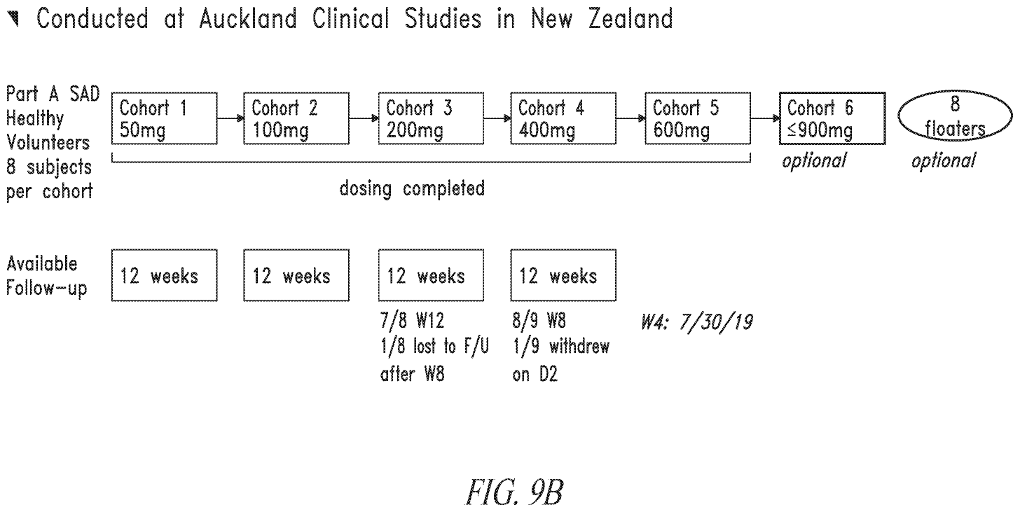

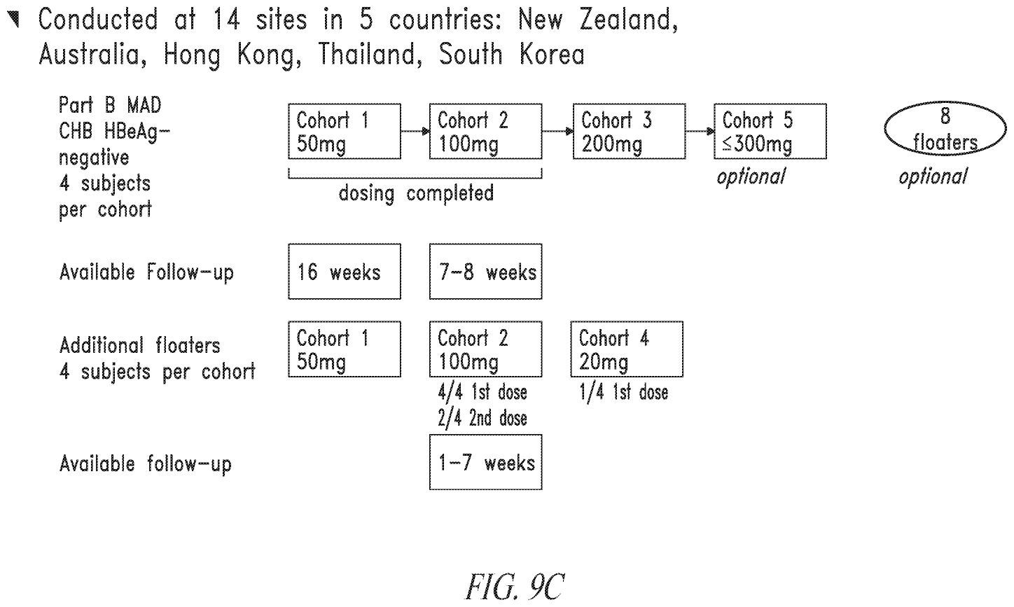

FIG. 9A illustrates the Part A, Part B, and Part C study design at the time dosing was completed for Part A cohorts 1 through 5 (50 mg, 100 mg, 200 mg, 400 mg, 600 mg) and for Part B cohorts 1 through 2 (50 mg, 100 mg). FIG. 9B illustrates the Part A completed dosing for cohorts 1 through 5, and the withdrawal of subjects in the different cohorts. FIG. 9C depicts the Part B completed dosing for cohorts 1 through 2, and the withdrawal of subjects in the different cohorts.

The preliminary demographic data for subjects included in Parts A and B are shown in Table 9 below.

TABLE 9
Demographics for subjects enrolled in Parts A and B.

| | | | | Part B | |
|---|---|---|---|---|---|
| | | | | Cohorts 1, 2, 4 | |
| | | Part A | | N = 13 | |
| | | Cohorts 1-5 | | (10 active, | |
| | | N = 41 | | 3 placebo) | |
| Gender | Male | 13 | (31.7%) | 11 | (84.6%) |
| | Female | 28 | (68.3%) | 2 | (15.4%) |
| Race/ | White | 21 | (51.2%) | 1 | (7.7%) |
| Ethnicity | Asian | 8 | (19.5%) | 11 | (84.6%) |

| | | | |
|---|---|---|---|
| Native Hawaiian/ | 3 | (7.3%) | 0 |
| Pacific Islander | | | |
| Other | 9 | (30.0%) | 1 (Maori) |
| | | | (7.7%) |
| Hispanic | 1 | (2.4%) | 0 |

| | | | |
|---|---|---|---|
| Other | Age mean (range) | 25.9 (19 | 43 (31 |
| | | to 41) | to 53) |
| | Baseline HBsAg | N/A | 3253 (547 |
| | mean (range) | | to 16,522) |
| | HBV genotype | N/A | unknown |

A summary of Adverse Events (AE) in from the preliminary analysis of the completed dosing portions of Parts A and B is presented in Table 10.

TABLE 10
Summary of Adverse Events.

| | | Part B |
|---|---|---|
| | | Cohorts 1, 2, 4 |
| | Part A | N = 13 |
| | Cohorts 1-5 | (10 active, |
| Number of Subjects with: | N = 41 | 3 placebo) |
| Any AEs | 32 (78%) | 4 (31%) |
| Grade 1 | 30 (73%) | 4 (31%) |
| Grade 2 | 2 (4.9%), URI | 0 |
| Grade 3 or 4 | 0 | 0 |
| Any treatment-emergent | 25 (61%) | 4 (31%) |
| adverse events (TEAEs) | | |
| (4 weeks post-dose) | | |
| Any treatment-related AEs | 3 (7.3%), all grade 1 | 1 (7.7%), grade 1 |
| (all occurred 4 weeks | Headache | Injection site |
| post-dose) | Injection site | pain |
| | tenderness | |
| | Abdominal | |
| | discomfort | |
| Injection site reactions | 6 (15%) | 1 (7.7%) |
| | 5/6 had injection | |
| | site pain | |
| | 1/6 bruising | |

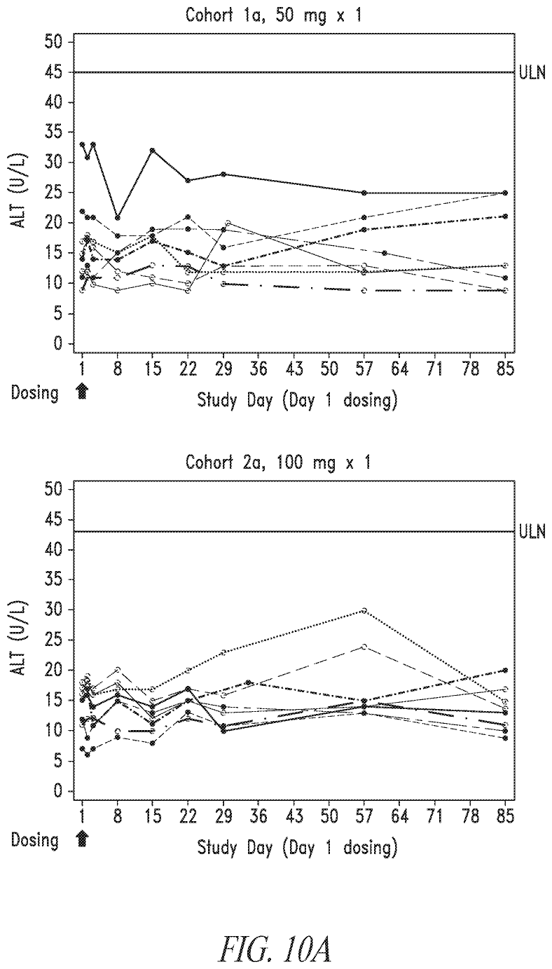

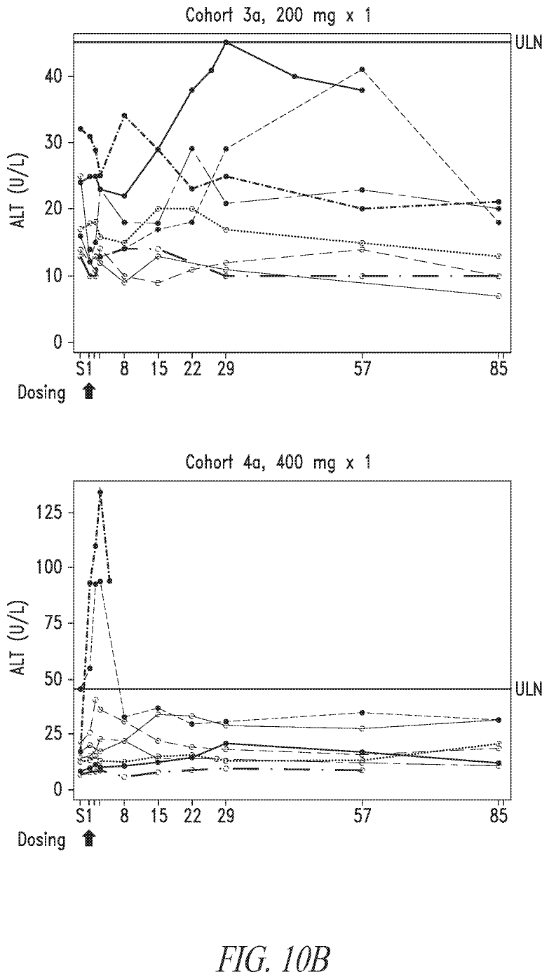

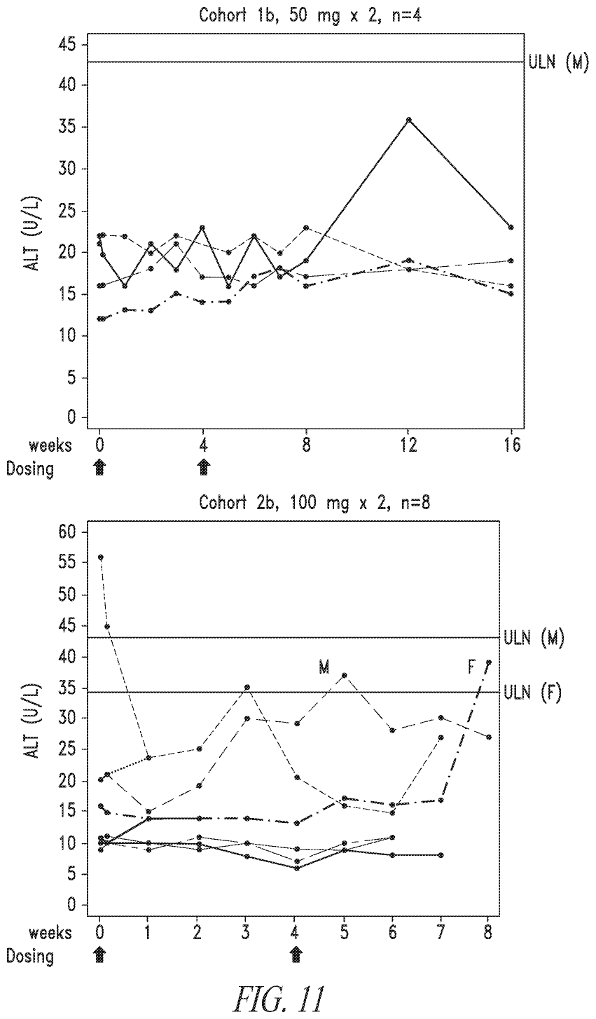

Subjects in Parts A and B showed no significant abnormalities in laboratory values, hyperbilirubinemia, or elevated INR. Some subjects in Parts A and B exhibited abnormalities in their liver function lab values (FIGS. 10A, 10B, and 11). Two out of 41 subjects in Part A had ALT elevations on Day 1 prior to dosing (normal ALT at screening). In Part B, 1 out of 12 subjects showed grade 1 ALT (39 U/L, 1.1×ULN) and AST (50 U/L, 1.5×ULN) elevations at Week 8. One subject in cohort 3a (200 mg) with ALT at the upper limit of normal on day 29 was associated with strenuous exercise and high creatinine kinase (CK: 5811 U/L). Two subjects in cohort 4a (400 mg) had ALT above the upper limit of normal on Day 1 prior to dosing. One admitted to strenuous exercise, had high CK of 20,001 U/L, and withdrew on day 2 unrelated to adverse events. The second subject with ALT elevation resolved by Day 8 without intervention. As shown in FIG. 11, one female subject in cohort 2b (100 mg) showed grade 1 ALT elevation at Week 8.

Subjects from Part B showed a decrease in HBsAg over time in the active groups of cohort 1 and 2. FIG. 12A depicts the change in HBsAg in cohorts 1b (50 mg) and 2b (100 mg) for subjects receiving HBV02 or placebo. FIG. 12B depicts the change in HBsAg in cohorts 1b and 2b for only subjects receiving HBV02. In cohort 4b (the 20 mg ×2 group), a subject had a 0.47 log decline 2 weeks after the first dose.

FIG. 12C shows the mean change in HBsAg in cohorts 1b and 2b from Day 1 to Week 4 or Week 20 (depending on cohort), following administration of HBV02, with 3 subjects with chronic HBV infection (HBeAg negative) having received 50 mg of HBV02 on Day 1 and Day 28, and six subjects having received 100 mg on Day 1. In the 50 mg cohort, the average decline in HBsAg at Week 12, after two doses, was 1.5 log₁₀, or approximately 30-fold reduction. All subjects in this cohort reached their apparent maximal decline in HBsAg, which has ranged from 0.6 to 2.2 log₁₀. In the 100 mg cohort, all subjects had reached Week 4, where an average decline of 0.7 log₁₀, or approximately six-fold reduction, was observed after one dose.

Among the 10 HBeAg-negative subjects in Part B, 7 subjects were good responders, showing a 0.29 to 0.95-log decline in HBsAg 2 weeks after the first dose (20, 50, or 100 mg). Two out of 10 were intermediate responders, showing a 0.06 to 0.21-log decline in HBsAg 2 weeks after the first dose of 20, 50, or 100 mg. Finally, one of the 10 subjects was a “non-responder,” showing a 0.16-log increase in HBsAg 2 weeks after the first dose. Possible reasons for the presence of intermediate and non-responders include: dose response, pharmacokinetics, viral resistance, and host factors.

HBV02 was well-tolerated among the subjects. Single doses ranging from 50 to 600 mg were well tolerated in healthy volunteer subjects. Two doses ranging from 50 to 100 mg were well tolerated in HBeAg-negative subjects. There was a high interpatient variability in HBsAg decline, with a rebound 12 weeks after the last dose.

iv. Demographics and Baseline Characteristics—Parts A, B, and C

The demographics and baseline characteristics of subjects in Parts A, B, and C are shown in Table 11, Table 12, and Table 13, respectively. All subjects in Parts B and C were NRTI suppressed and had FibroScan ≤8.5 kPa or Metavir F0/F1/F2.

TABLE 11
Demographics and baseline characteristics of subjects in Part A (healthy volunteers).

| | HBV02 | | | | | | | | | | | | | | | |
|---|---|---|---|---|---|---|---|---|---|---|---|---|---|---|---|---|
| | 50 mg | | 100 mg | | 200 mg | | 400 mg | | 600 mg | | 900 mg | | Overall | | Placebo | |
| | n = 6 | | n = 6 | | n = 6 | | n = 7^{a} | | n = 6 | | n = 6 | | n = 37 | | n = 12 | |
| Mean age, | 25 | (3) | 23 | (4) | 27 | (4) | 24 | (4) | 29 | (6) | 33 | (10) | 27 | (6) | 27 | (7) |
| y (SD) | | | | | | | | | | | | | | | | |
| Male sex, | 0 | | 2 | (33) | 3 | (50) | 0 | | 3 | (50) | 3 | (50) | 11 | (30) | 7 | (58) |
| n (%) | | | | | | | | | | | | | | | | |
| Mean weight, | 62 | (12) | 63 | (7) | 75 | (5) | 65 | (10) | 72 | (8) | 72 | (12) | 68 | (10) | 76 | (10) |
| kg (SD) | | | | | | | | | | | | | | | | |
| Mean BMI, | 23 | (5) | 23 | (3) | 24 | (2) | 25 | (4) | 26 | (1) | 26 | (4) | 25 | (3) | 24 | (2) |
| kg/m² (SD) | | | | | | | | | | | | | | | | |
| Race, n (%) | | | | | | | | | | | | | | | | |
| Asian | 2 | (33) | 3 | (50) | 0 | | 0 | | 2 | (33) | 1 | (17) | 8 | (22) | 1 | (8) |
| White | 2 | (33) | 2 | (33) | 5 | (83) | 5 | (71) | 3 | (50) | 3 | (50) | 20 | (54) | 8 | (67) |
| Other | 1 | (17) | 1 | (17) | 1 | (17) | 1 | (14) | 1 | (17) | 2 | (33) | 6 | (16) | 1 | (8) |

SD = standard deviation.
^{a}includes replacement volunteer

TABLE 12
Demographics and baseline characteristics of
subjects in Part B (HBeAg-negative patients).

| | HBV02 | | | | | | | | | | | |
|---|---|---|---|---|---|---|---|---|---|---|---|---|
| | 20 mg | | 50 mg | | 100 mg | | 200 mg | | Overall | | Placebo | |
| | n = 3 | | n = 6 | | n = 6 | | n = 3 | | n = 18 | | n=6 | |
| Mean age, | 40 | (9) | 43 | (11) | 45 | (6) | 55 | (4) | 45 | (9) | 44 | (7) |
| y (SD) | | | | | | | | | | | | |
| Male sex, | 2 | (67) | 5 | (83) | 5 | (83) | 0 | | 12 | (67) | 3 | (50) |
| n (%) | | | | | | | | | | | | |
| Race, n (%) | | | | | | | | | | | | |
| Asian | 3 | (100) | 5 | (83) | 5 | (83) | 3 | (100) | 16 | (89) | 6 | (100) |
| White | 0 | | 0 | | 1 | (17) | 0 | | 1 | (6) | 0 | |
| Other | 0 | | 1 | (17) | 0 | | 0 | | 1 | (6) | 0 | |
| Mean log₁₀ | 3.3 | (0.3) | 3.3 | (0.5) | 3.4 | (0.5) | 3.3 | (0.4) | 3.3 | (0.4) | 3.5 | (0.4) |
| HBsAg (SD) | | | | | | | | | | | | |

SD = standard deviation.

TABLE 13
Demographics and baseline characteristics of
subjects in Part C (HBeAg-positive patients).

| | HBV02 | | | | | | | |
|---|---|---|---|---|---|---|---|---|
| | 50 mg | | 200 mg | | Overall | | Placebo | |
| | n = 3 | | n = 3 | | n = 6 | | n = 2 | |
| Mean age, | 35 | (10) | 34 | (13) | 34 | (10) | 59 | (8) |
| y (SD) | | | | | | | | |
| Male sex, | 1 | (33) | 2 | (67) | 3 | (50) | 1 | (50) |
| n (%) | | | | | | | | |
| Race, n (%) | | | | | | | | |
| Asian | 3 | (100) | 3 | (100) | 6 | (100) | 2 | (100) |
| White | 0 | | 0 | | 0 | | 0 | |
| Other | 0 | | 0 | | 0 | | 0 | |
| Mean log₁₀ | 3.5 | (0.3) | 3.9 | (0.6) | 3.7 | (0.5) | 3.2 | (0.3) |
| HbsAg (SD) | | | | | | | | |

SD = standard deviation.

v. Safety and Tolerability—Results from Parts A, B, and C

Preliminary data were obtained from Parts A, B, and C based on 37 healthy volunteers that received HBV02; 12 healthy volunteers that received placebo; 24 patients with chronic HBV on NRTIs that received HBV02; and 8 patients with chronic HBV on NRTIs that received placebo. HBV02 was generally well-tolerated.

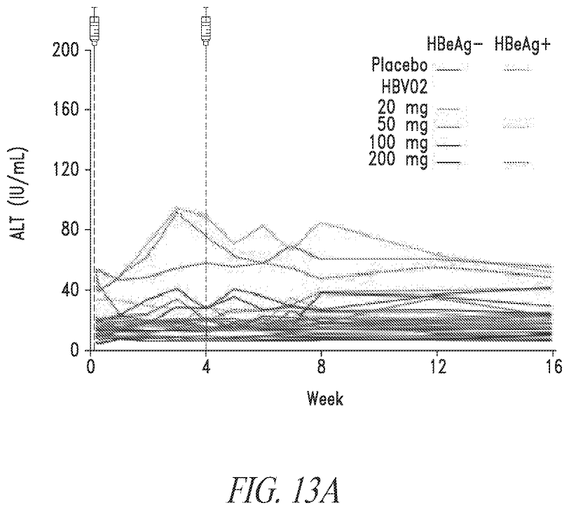

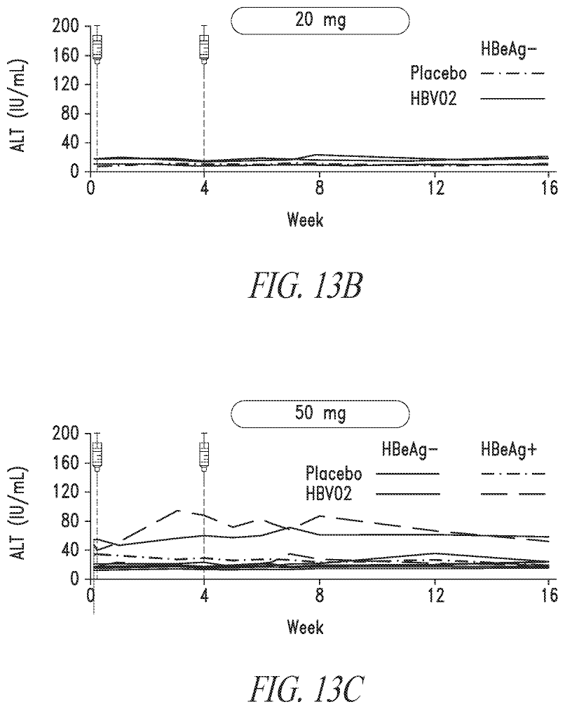

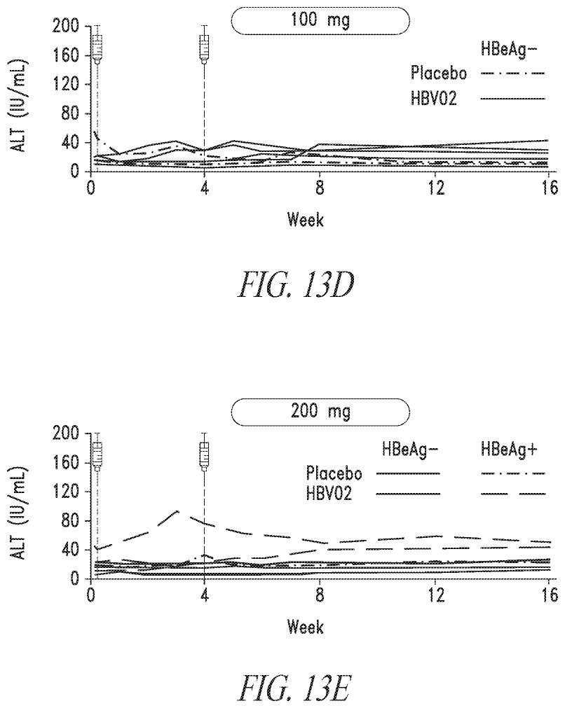

Across healthy volunteers and chronic HBV patients, HBV02 was generally well-tolerated in healthy volunteers given as a single dose up to 900 mg and in patients given as two doses of 20 mg, 50 mg, 100 mg, or 200 mg each dose. No clinically significant alanine transaminase (ALT) abnormalities, which are a marker of liver inflammation, were observed through Week 16 for chronic HBV patients (Parts B and C) (FIGS. 13A-13E). No Grade ≥2 ALT elevations, levels of bilirubin >ULN, or clinically relevant changes or trends in other laboratory parameters, vital signs, or ECGs were observed.

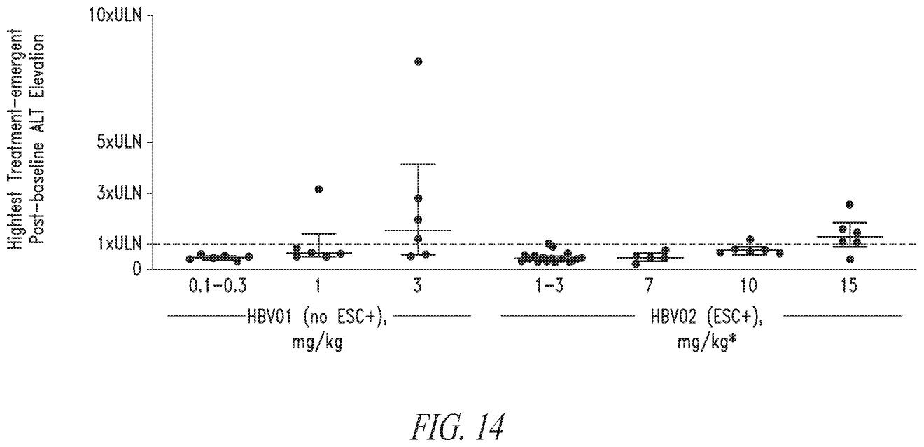

For Part A, no post-baseline ALT elevations to >ULN were associated with increases in bilirubin >ULN. No changes in functional status of the liver (e.g., albumin, coagulation parameters) or clinical signs/symptoms of hepatic dysfunction were observed in any HBV02-treated subject. Transient ALT elevations were observed with HBV02 in 1/6 (17%) and 4/6 (67%) subjects after a single dose of 1 and 3 mg/kg, respectively. These elevations were asymptomatic and not accompanied by hyperbilirubinemia. In contrast, no ALT elevations potentially related to HBV02 were observed with single doses of HBV02 ranging from 50-600 mg (˜0.8 to 10 mg/kg). In the Part A 900 mg (˜15 mg/kg) cohort, mild, asymptomatic Grade 1 ALT elevations, with no associated changes in bilirubin, were observed in a subset of subjects (5/6 of subjects having ALT elevations 1.1-2.6×ULN). The ALT levels for subjects in Part A, including relative to subjects administered HBV01 (a similar siRNA lacking the GNA modification), are shown in FIG. 14. These results suggest that incorporating ESC+ technology (providing enhanced stability and minimized off-target activity through encorporation of a GNA modification) decreases the propensity of siRNAs to cause ALT elevations in healthy volunteers at dose levels anticipated to be clinically relevant.

No dose-related trends in the frequency of adverse events were observed. The majority of treatment emergent adverse events that were preported were mild in severity, and no patients discontinued due to an adverse event. The most common adverse event was headache (6/24, 25%). Three Grade 3 adverse events of upper-respiratory tract infection, chest pain, and low phosphate levels in the blood were reported, but were not considered to be related to HBV02. There was a single Grade 3 adverse event of hypophosphatemia observed in a patient on tenofovir disoproxil fumarate. Two serious adverse events, or SAEs, were reported, both in Part B. The first, a Grade 2 headache, resolved with intravenous fluids and non-opioid pain medications. This patient had additional symptoms of fever, nausea, vomiting, and dehydration, assessed as being consistent with a viral syndrome. The second SAE, a Grade 4 depression, occurred over 50 days after the last drug dose was administered, and was assessed to be unrelated to HBV02 treatment.

A summary of the treatment emergent adverse events is shown in Table 14.

TABLE 14
Summary of treatment emergent adverse events (AE).

| | | |
|---|---|---|
| Patients, n (%) | HBV02 n = 24 | Placebo n = 8 |

| | | | |
|---|---|---|---|
| Any AE | 13 | (54) | 2 (25) |
| Treatment- | 5 | (21) | 0 |
| related AE | | | |
| Grade ≥3 AE | 1 | (4) | 0 |
| Serious AE | 1 | (4) | 0 |

vi. Pharmacokinetics—Results from Part A

Preliminary pharmacokinetic (PK) data from the first-in-human Phase 1 randomized, blinded, placebo-controlled, dose ranging study of HBV02 in healthy volunteers were analyzed. Plasma samples were assessed for six single ascending dose cohorts of eight subjects (6:2 active:placebo) that received a single subcutaneous (SC) dose of HBV02 ranging from 50 to 900 mg.

Eligibility criteria included the following: Age 18 to 55 y; Body mass index (BMI) 18.0−≤32 kg/m²; CLcr <90 mL/min (Cockcroft-Gault); and no clinically significant ECG abnormalities or clinically significant chronic medical condition.

Intensive plasma and urine PK samples were collected for 1 week. Serial plasma samples were collected over 24 hr, at 48 hr, and 1 week post dose. Pooled urine samples were collected over 24 hr, and single void samples were collected at 48 hr and 1 week postdose. Concentrations of HBV02 and (N-1)3′ HBV02 antisense metabolite in plasma and urine were measured using validated liquid chromatography tandem mass spectroscopy assays (lower limit of quantitation (LLOQ) of 10 ng/mL in plasma and urine). PK parameters were estimated using standard noncompartmental methods in WinNonlin®, V6.3.0 (Certara L.P., Princeton, N.J.). AS(N-1)3′ HBV02, the primary circulating metabolite with equal potency to HBV02, is formed by the loss of one nucleotide from the 3′ end of the antisense strand of HBV02.

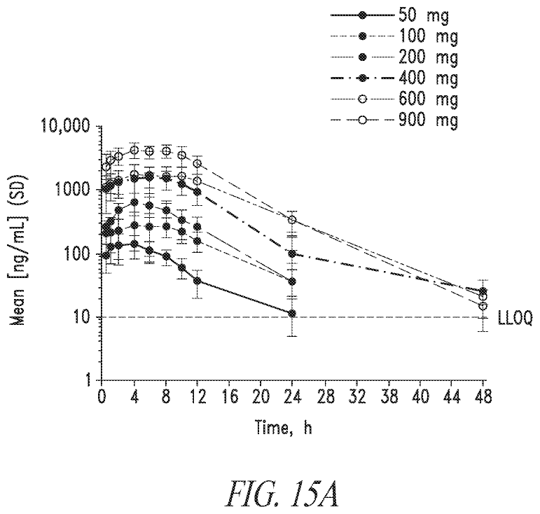

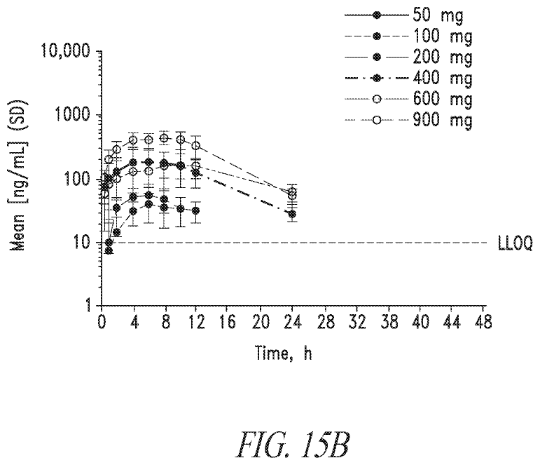

FIG. 15A and FIG. 15B show plasma concentration vs. time profiles for HBV02 and AS(N-1)3′ HBV02, respectively, after a single SC dose in healthy volunteers. HBV02 exhibited linear kinetics in plasma after SC injection. HBV02 was absorbed after SC injection with median Tₘₐₓ of 4-8 hours. HBV02 was not measurable in plasma after 48 hours for any subject, consistent with rapid GalNAc-mediated liver uptake; the median apparent elimination half-life (t_{1/2}) ranged from 2.85-5.71 hours. The short plasma half-life likely represents the distribution half life (see Agarwal S, et al., Clin Pharmacol Ther. 2020 Jan. 29, doi: 10.1002/cpt.1802). A rapid conversion of HBV02 to the (N-1)3′ metabolite, referred to as AS(N-1)3′ HBV02, was observed. AS(N-1)3′ HBV02 had a median Tₘₐₓ of 2-10 hr, was quantifiable only at doses ≥100 mg, and had concentrations generally ˜10 fold lower compared to HBV02.

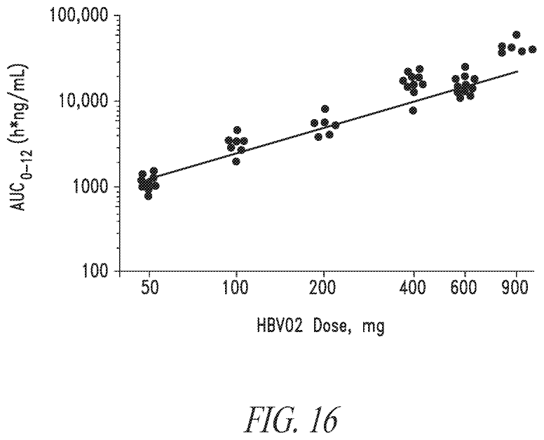

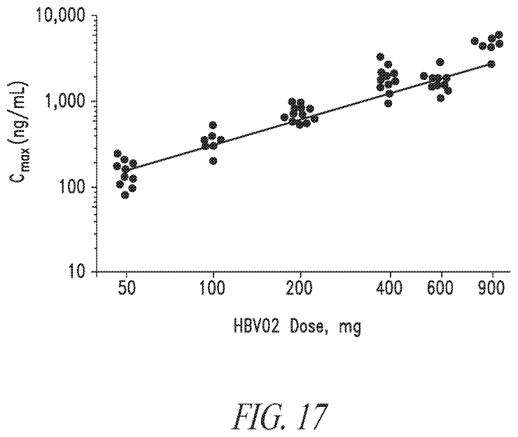

HBV02 plasma exposures (AUC₀₋₁₂ and Cₘₐₓ) appeared to increase in a dose proportional manner up to 200 mg and exhibited slightly greater than dose-proportional increase at doses above 200 mg (FIG. 16; FIG. 17; Table 15). Following a single SC dose of HBV02 of 50 to 900 mg, plasma area under the curve (AUCₗₐₛₜ) and mean-maximum concentrations (Cₘₐₓ) increased with dose with mean exposures ranging between 786 to 74,700 ng*hr/mL and 77.8 to 6010 ng/mL, respectively. A similar trend was observed for AS(N-1)3′ HBV02. These results indicate transient saturation of ASGPR-mediated hepatic uptake of HBV02 resulting in higher circulating concentrations at higher doses (see Agarwal et al., 2020, supra).

TABLE 15
Fold-change between HBV02 plasma exposure and dose.

| | | | |
|---|---|---|---|
| Dose Range | Fold Change | AUC₀₋₁₂ | Cₘₐₓ |

| | | | | |
|---|---|---|---|---|
| 50-200 | mg | 4 | 4.57 | 4.59 |
| 200-900 | mg | 4.5 | 8.08 | 7.05 |

Interpatient variability in HBV02 plasma PK parameters was generally low (˜30%).

The most prevalent active metabolite (˜12%), AS(N-1)3′ HBV02, is equally potent as HBV02. AS(N-1)3′ HBV02 was detectable in plasma in 0/6 subjects at 50 mg, 3/6 subjects at 100 mg, and in all subjects at 200, 400, 600, and 900 mg. The PK profile of the metabolite was similar to HBV02 with AUCₗₐₛₜ and Cₘₐₓ values of AS(N-1)3′ HBV02 in plasma ≤11% of HBV02.

AUC₀₋₁₂ and Cₘₐₓ of AS(N-1)3′ HBV02 in plasma were ≤11% of total drug related material.

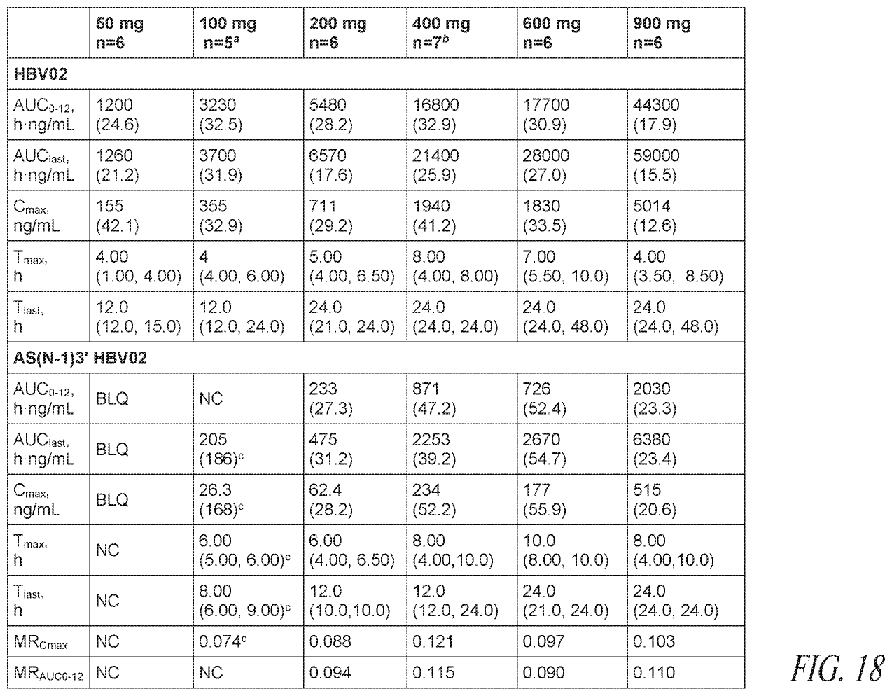

A summary of the plasma PK parameters for HBV02 and AS(N-1)3′ HBV02 observed after a single SC dose in healthy volunteers is shown in FIG. 18.

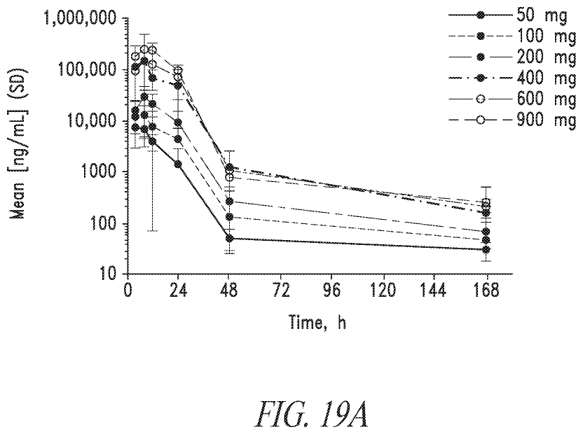

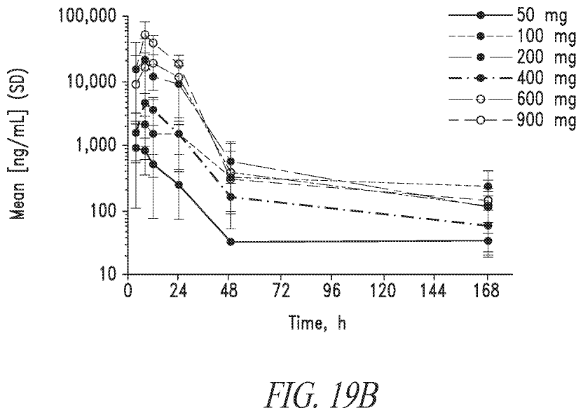

Urine concentration vs. time profiles for HBV02 and AS(N-1)3′ HBV02 are shown in FIGS. 19A and 19B, respectively. Low concentrations of HBV02 and AS(N-1)3′ HBV02 were detected in urine through the last measured time-point at 1 week post-dose in all cohorts. The PK profile of HBV02 in urine mirrored that of plasma where calculable.

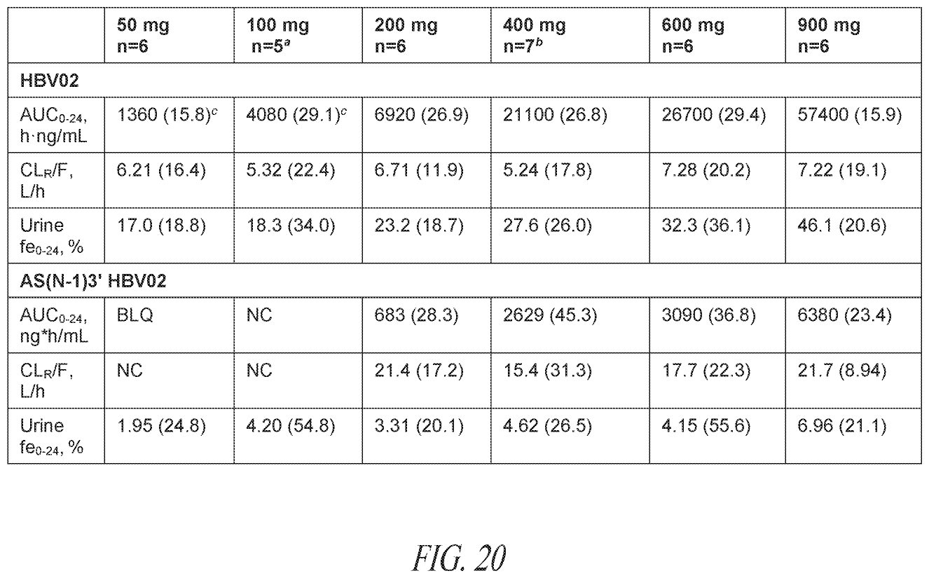

A summary of the urine PK parameters for HBV02 and AS(N-1)3′ HBV02 in healthy volunteers is shown in FIG. 20. Approximately 17-46% and 2-7% of the administered dose (50-900 mg) was excreted in urine as unchanged HBV02 and AS(N-1)3′ HBV02, respectively, over the first 24-hr period. The fraction of HBV02 excreted into urine over 24 hr post-dose increased with dose level. This likely resulted from a rate of HBV02 hepatic uptake by ASGPR far in excess of renal elimination (see Agarwal et. al, 2020, supra), and mirrors greater than dose proportional increases in plasma HBV02. The renal clearance of HBV02 approached glomerular filtration rate.

These preliminary data show that HBV02 demonstrated favorable PK properties in healthy volunteers.

vii. Efficacy—Results from Parts B and C

Preliminary data were obtained from B and C based on 24 patients with chronic HBV on NRTIs that received HBV02; and 8 patients with chronic HBV on NRTIs that received placebo. Initial data demonstrated substantial reductions in HBsAg in patients at doses ranging from 20 mg to 200 mg.

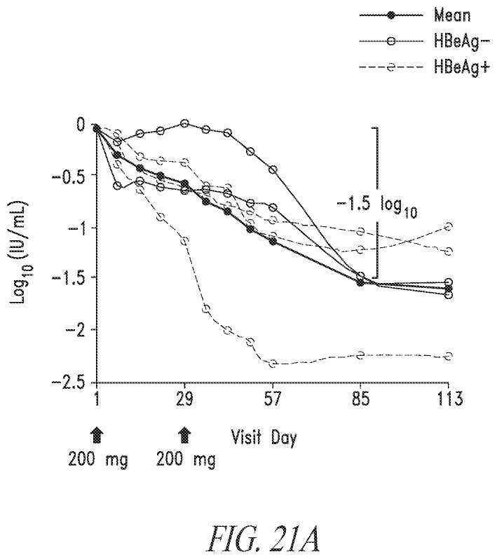

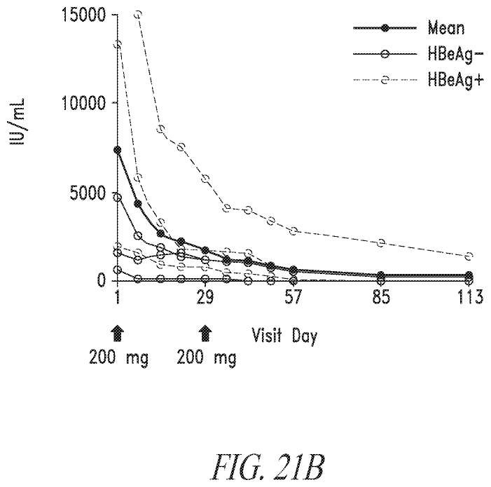

The biologic activity of HBV02 was assessed by declines in HBsAg. The activity of HBV02 through Week 16 in the 200 mg cohorts of Part B, HBeAg-negative, and Part C, HBeAg-positive, is shown in FIGS. 21A and 21B. For Parts B and C, the average baseline HBsAg levels were 3.3 log₁₀ IU/mL and 3.9 log₁₀ IU/mL, respectively. The average decline in HBsAg across HBeAg-negative and HBeAg-positive subjects at Week 16 was 1.5 log₁₀, or an approximately 32-fold reduction. The declines observed in HBsAg at Week 16 ranged from 0.97 log₁₀ to 2.2 log₁₀, or an approximately nine to 160-fold reduction, after two 200 mg doses of HBV02 given four weeks apart. The average HBsAg level at Week 16 was 314 IU/mL, with half of patients achieving HBsAg values <100 IU/mL and 5/6 achieving HBsAg values <1000 IU/mL.

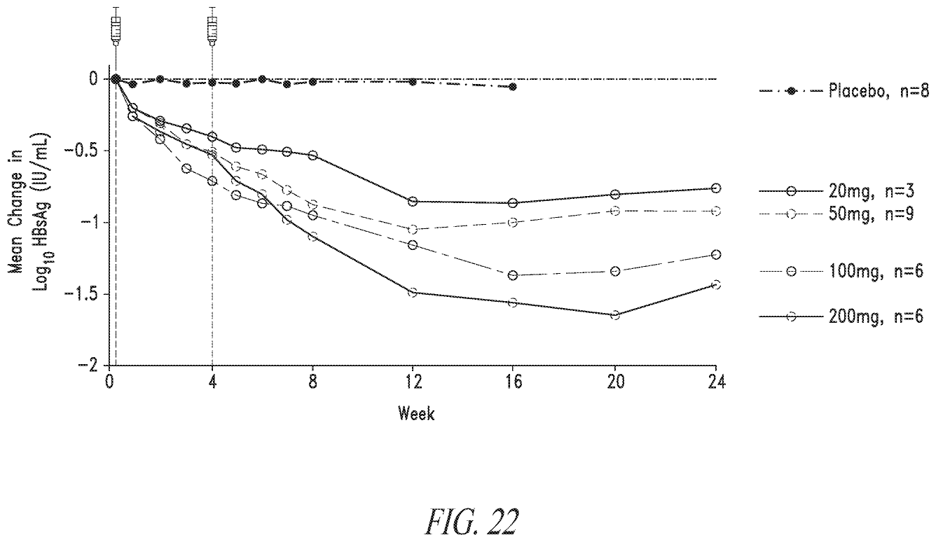

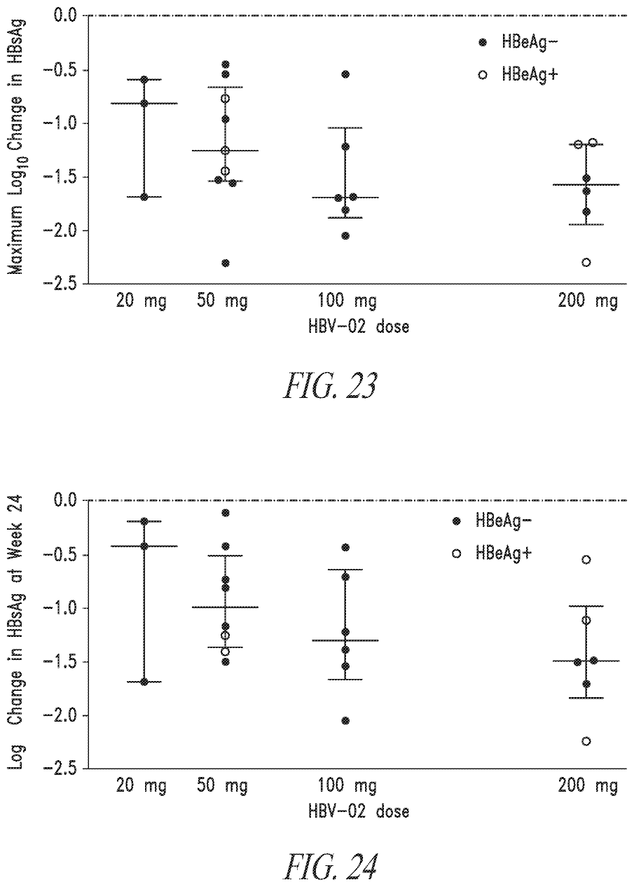

The change in HBsAg from baseline through Week 16, by dose, is shown in FIG. 22. The percent of patients having HBsAg levels <100 IU/mL at Week 24 was 33% for patients receiving 20 mg HBV02, 44% for patients receiving 50 mg HBV02, 50% for patients receiving 100 mg HBV02, and 50% for patients receiving 200 mg HBV02. Individual maximum HBsAg change from baseline is shown in FIG. 23. Similar reductions were observed in HBeAg-positive and HBeAg-negative patients. At Week 24, the mean change in HBsAg observed in patients administered HBV02 at 20 mg, 50 mg, 100 mg, and 200 mg was 31 0.76 log₁₀, −0.93 log₁₀, −1.23 log₁₀, and −1.43 log₁₀, respectively. All 6 patients who received 2 doses of 200 mg achieved ≥1.0 log₁₀ decline in HBsAg. Individual HBsAg change from baseline at Week 24 is shown in FIG. 24, indicating a dose-dependent durability in HBsAg decline.

These results show that HBV02 was well tolerated, with no safety signals observed. Dose-dependent HBsAg reductions in HBeAg-negative and HBeAg-positive patients were observed across the dose range of 20 to 200 mg of HBV02 (2 doses delivered), which were durable at the higher doses for at least 6 months. Similar HBsAg reductions were observed in both HBeAg-negative and HBeAg-positive patients, demonstrating that HBV02 can decrease HBsAg in patients regardless of the stage of their disease. All patients who received 2 doses of 200 mg achieved a ≥1−log₁₀ reduction in HBsAg, and at Week 24, the mean decline in HBsAg was −1.43 log₁₀. Overall, these results support the potential of HBV02 as a backbone for a finite treatment regimen aimed at functional cure of chronic HBV infection. In particular, the ability of HBV02 to result in substantial declines in HBsAg after only two doses suggests that HBV02 has the potential to play an important role in the functional cure of chronic HBV.

While specific embodiments have been illustrated and described, it will be readily appreciated that the various embodiments described above can be combined to provide further embodiments, and that various changes can be made therein without departing from the spirit and scope of the invention.

All of the U.S. patents, U.S. patent application publications, U.S. patent applications, foreign patents, foreign patent applications, and non-patent publications referred to in this specification or listed in the Application Data Sheet, including U.S. Provisional Patent Applications Nos. 62/846,927 filed May 13, 2019, 62/893,646 filed Aug. 29, 2019, 62/992,785 filed Mar. 20, 2020, 62/994,177 filed Mar. 24, 2020, and 63/009,910 filed Apr. 14, 2020, and International Application No. PCT/US2020/032525, filed May 12, 2020, are incorporated herein by reference, in their entirety, unless explicitly stated otherwise. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating chronic hepatitis B virus (HBV) infection or an HBV-associated disease in a subject in need thereof, comprising: administering to the subject an siRNA and a pegylated interferon-alpha (PEG-IFNα), wherein two or more doses of at least 200 mg of the siRNA are administered, wherein the siRNA has a sense strand comprising 5′-gsusguGfcAfCfUfucgcuucacaL96-3′ (SEQ ID NO:5) and an antisense strand comprising 5′-usGfsuga(Agn)gCfGfaaguGfcAfcacsusu-3′ (SEQ ID NO:6),
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
(Agn) is adenosine-glycol nucleic acid (GNA);
s is a phosphorothioate linkage; and
L96 is N-[tris(GalNAc-alkyl)-amidodecanoyl)]-4-hydroxyprolinol.

2. (canceled)

3. The method of claim 1, wherein:
(a) the siRNA and PEG-IFNα are administered to the subject over the same time period;
(b) the siRNA is administered to the subject for a period of time before the PEG-IFNα is administered to the subject;
(c) the PEG-IFNα is administered to the subject for a period of time before the siRNA is administered to the subject;
(d) the subject has been administered PEG-IFNα prior to the administration of the siRNA; or
(e) the subject is administered PEG-IFNα during the same period of time that the subject is administered the siRNA.

4.-8. (canceled)

9. The method of claim 1, further comprising administering to the subject a nucleoside/nucleotide reverse transcriptase inhibitor (NRTI), wherein:
(a) the subject has been administered a NRTI prior to the administration of the siRNA:
(b) the subject has been administered a NRTI for at least 2 months or at least 6 months prior to the administration of the siRNA;
(c) the subject is administered a NRTI during the same period of time that the subject is administered the siRNA; or
(d) the subject is subsequently administered a NRTI.

10.-30. (canceled)

31. The method of claim 1, wherein at least one dose of the siRNA is 200 mg, 250 mg, 300 mg, 400 mg, or 450 mg.

32. The method of claim 1, wherein the two or more doses are administered weekly, or more than one dose is administered with each dose separated by 2, 3, or 4 weeks.

33. The method of claim 1, wherein two, three, four, five, six, or more doses of the siRNA are administered with each dose separated by 1, 2, 3, or 4 weeks.

34. The method of claim 1, wherein the method further comprises:
administering to the subject a nucleoside/nucleotide reverse transcriptase inhibitor (NRTI);
wherein the subject is HBeAg negative or HBeAg positive.

35. (canceled)

36. The method of claim 1, wherein six 200-mg doses, or two 400-mg doses of the siRNA are administered.

37. (canceled)

38. The method of claim 1, wherein the siRNA is administered via subcutaneous injection.

39. The method of claim 38, wherein administering the siRNA comprises administering 1, 2, or 3 subcutaneous injections per dose.

40. The method of claim 1, wherein the dose of the PEG-IFNα is 50 μg, 100 μg, 150 μg, 180 μg, or 200 μg.

41. The method of claim 1, wherein the PEG-IFNα is administered weekly.

42. The method of claim 1, wherein the PEG-IFNα is administered via subcutaneous injection.

43. The method of claim 9, wherein the NRTI is tenofovir, tenofovir disoproxil fumarate (TDF), tenofovir alafenamide (TAF), lamivudine, adefovir, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, ganciclovir, besifovir (ANA-380/LB-80380), or tenofvir-exaliades (TLX/CMX157).

44.-47. (canceled)

48. The method of claim 1, wherein the subject is HBeAg negative.

49. The method of claim 1, wherein the subject is HBeAg positive.

50. A kit comprising:
a pharmaceutical composition comprising the siRNA of claim 1, and a first pharmaceutically acceptable excipient; and
a pharmaceutical composition comprising PEG-IFNα, and a second pharmaceutically acceptable excipient.

51. The kit according to claim 50, further comprising a NRTI, and a third pharmaceutically acceptable excipient.

52. The method of claim 1, wherein the HBV-associated disease is a Hepatitis D (HDV) infection.

53. The method of claim 1, wherein the subject has a HDV infection.


SEQUENCE LISTING (10 sequences)

SEQ ID NO: 1 - DNA, 3182 bp, Hepatitis B Virus
  Hepatitis B Virus genome, NCBI Reference Sequence NC_003977.2
aattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtgg

SEQ ID NO: 2 - DNA, 18 bp, Hepatitis B Virus
  Hepatitis B Virus genome, nucleotides 1579-1597 of NC_003977.2
gtgtgcacttcgcttcac

SEQ ID NO: 3 - RNA, 19 nt, Artificial Sequence
  HBV02, sense strand
gugugcacuucgcuucaca

SEQ ID NO: 4 - RNA, 21 nt, Artificial Sequence
  HBV02, antisense strand
ugugaagcgaagugcacacuu

SEQ ID NO: 5 - RNA, 19 nt, Artificial Sequence
  HBV02 modified, sense strand
gugugcacuucgcuucaca

SEQ ID NO: 6 - RNA, 21 nt, Artificial Sequence
  HBV02 modified, antisense strand
ugugaagcgaagugcacacuu

SEQ ID NO: 7 - protein, 16 aa, Artificial Sequence
  Membrane translocation sequence-containing peptide RFGF
AAVALLPAVLLALLAP

SEQ ID NO: 8 - protein, 11 aa, Artificial Sequence
  Membrane translocation sequence-containing peptide RFGF analogue
AALLPVLLAAP

SEQ ID NO: 9 - protein, 13 aa, Artificial Sequence
  HIV Tat protein
GRKKRRQRRRPPQ

SEQ ID NO: 10 - protein, 15 aa, Artificial Sequence
  Drosophila Antennapedia protein
RQIKIWFQNRRMKWK